(19) United States
(12) Patent Application Publication
(10) Pub. No.: US 2022/0161021 A1
(43) Pub. Date: May 26, 2022

(54) MECHANICAL CIRCULATORY SUPPORT SYSTEM WITH INSERTION TOOL

(71) Applicant: Kardion GmbH, Stuttgart (DE)

(72) Inventors: Marvin Mitze, Stuttgart (DE); Hans Christof, Unterensingen (DE); Vladimir Popov, Stuttgart (DE); Martin Schwarz, Wangen (DE); Leon Wenning, Stuttgart (DE); Johannes Bette, Balingen (DE); Attila Fabiunke, Karlsruhe (DE); Julian Görries, Stuttgart (DE); Jan Schöfer, Stuttgart (DE); Valentin Rex, Aachen (DE); Johannes Berner, Stuttgart (DE); Johannes Ferch, Esslingen (DE); Hans-Baldung Luley, Stuttgart (DE); Tom Döhring, Esslingen (DE); Jens Burghaus, Stuttgart (DE); Inga Schellenberg, Stuttgart (DE); Hardy Baumbach, Stuttgart (DE); Annika Bach, Leutenbach (DE); Ingo Stotz, Ditzingen (DE); Julian Kassel, Boblingen (DE); Armin Schuelke, Aidlingen (DE); Stefan Henneck, leonberg (DE); David Minzenmay, Stuttgart (DE); Thomas Alexander Schlebusch, Renningen (DE); Tobias Schmid, Stuttgart (DE); Tjalf Pirk, Stuttgart (DE); Martina Budde, Karlsruhe (DE); Ricardo Ehrenpfordt, Korntal-Munchingen (DE); Marc Schmid, Stuttgart (DE); Ahmad Mansour, Weil der Stadt (DE); niko Baeuerle, Bad Liebenzell (DE); Peter Wassermann, Weil der Stadt (DE); Fabian Eiberger, Gerlingen (DE); Kenneth M. Martin, Woodside, CA (US); Thomas Friedrich, Dresden (DE); Mario Heintze, Dresden (DE)

(21) Appl. No.: 17/530,409

(22) Filed: Nov. 18, 2021

Related U.S. Application Data
(60) Provisional application No. 63/229,436, filed on Aug. 4, 2021.
(60) Provisional application No. 63/116,686, filed on Nov. 20, 2020.
(60) Provisional application No. 63/116,616, filed on Nov. 20, 2020.

(51) Int. Cl. A61M 60/839; A61M 60/465; A61M 60/216; A61M 60/827; A61M 60/419; A61M 60/89; A61M 60/806; A61M 60/865

(52) U.S. Cl. CPC A61M 60/839; A61M 60/465; A61M 60/216; A61M 60/827; A61M 2205/32; A61M 60/89; A61M 60/806; A61M 60/865; A61M 2205/3334; A61M 60/419

(57) ABSTRACT

A minimally invasive miniaturized percutaneous mechanical circulatory support system for transcatheter delivery of a pump to the heart that actively unloads the left ventricle by pumping blood from the left ventricle into the ascending aorta and systemic circulation. The pump may include a tubular housing, a motor, an impeller configured to be rotated by the motor. The impeller may be rotated by the motor, via a shaft with an annular polymeric seal around the shaft, or via a magnetic drive. The system may have an insertion tool having a tubular body and configured to axially movably receive the circulatory support device, and an introducer sheath configured to axially movably receive the insertion tool.

### INCORPORATION BY REFERENCE TO ANY PRIORITY APPLICATIONS

Any and all applications for which a foreign or domestic priority claim is identified in the Application Data Sheet as filed with the present application are hereby incorporated by reference under 37 CFR 1.57. For example, this application claims priority to U.S. Provisional Application No. 63/116,616, titled MECHANICAL LEFT VENTRICULAR SUPPORT SYSTEM FOR CARDIOGENIC SHOCK and filed on Nov. 20, 2020, U.S. Provisional Application No. 63/229,436, titled SEAL FOR A MECHANICAL CIRCULATORY SUPPORT DEVICE and filed on Aug. 4, 2021, and to U.S. Provisional Application No. 63/116,686, titled MECHANICAL CIRCULATORY SUPPORT SYSTEM FOR HIGH RISK CORONARY INTERVENTIONS and filed on Nov. 20, 2020, the entire contents of each of which is incorporated by reference herein in its entirety for all purposes and forms a part of this specification.

### BACKGROUND

Mechanical circulatory support systems may be used to assist with pumping blood during various medical procedures and/or as therapy for certain cardiac conditions. For example, cardiogenic shock (CS) is a common cause of mortality, and management remains challenging despite advances in therapeutic options. CS is caused by severe impairment of myocardial performance that results in diminished cardiac output, end-organ hypoperfusion, and hypoxia. Clinically this presents as hypotension refractory to volume resuscitation with features of end-organ hypoperfusion requiring immediate pharmacological or mechanical intervention. Acute myocardial infarction (MI) accounts for over about 80% of patients in CS.

As further example, percutaneous coronary intervention (PCI) is a non-surgical procedure to revascularize stenotic coronary arteries. PCI includes a variety of techniques, e.g. balloon angioplasty, stent implantation, rotablation and lithotripsy. A PCI is considered high risk if either the patient has relevant comorbidities (e.g. frailty or advanced age), the PCI per se is very complex (e.g. bifurcation or total occlusions) or hemodynamic status is challenging (e.g. impaired ventricular function).

Miniature, catheter-based intracardiac blood pumps have been developed for percutaneous insertion into a patient's body as an acute therapy for CS and for temporary assistance during PCI. However, existing solutions for pumps have various performance deficiencies such as, for example, inadequate blood flow, the requirement for ongoing motor purging within the pump, undesirably high hemolysis, and inadequate sensing of hemodynamic parameters. Thus, there remains a need for mechanical circulatory support systems with features that overcome these and other drawbacks.

### SUMMARY

The embodiments disclosed herein each have several aspects no single one of which is solely responsible for the disclosure's desirable attributes. Without limiting the scope of this disclosure, its more prominent features will now be briefly discussed. After considering this discussion, and particularly after reading the section entitled “Detailed Description,” one will understand how the features of the embodiments described herein provide advantages over existing systems, devices and methods for circulatory support systems.

The following disclosure describes non-limiting examples of some embodiments. For instance, other embodiments of the disclosed systems and methods may or may not include the features described herein. Moreover, disclosed advantages and benefits can apply only to certain embodiments and should not be used to limit the disclosure.

Various aspects and embodiments of mechanical circulatory support systems, devices and methods are described herein. The mechanical circulatory support systems, devices and methods may have one or more of any of the following features: a mechanical circulatory support system comprising a circulatory support catheter, comprising a circulatory support device carried by an elongate flexible catheter shaft, the circulatory support device comprising a tubular housing, a motor, an impeller configured to be rotated by the motor via a shaft, and an annular polymeric seal around the shaft, an insertion tool having a tubular body and configured to axially movably receive the circulatory support device, and an introducer sheath, having a tubular body and configured to axially movably receive the insertion tool; the introducer sheath comprises a hub on a proximal end of the introducer sheath, the hub having a lock for preventing axial movement of the insertion tool; the hub comprises one or more hemostatic valves; the tubular body of the insertion tool has sufficient collapse resistance to maintain patency when passed through the hemostatic valves of the introducer sheath; the catheter shaft comprises a visual marker spaced proximally from the circulatory support device such that visibility of the visual marker on a proximal side of the introducer sheath indicates the circulatory support device is located within the tubular body of the insertion tool; the system further comprises a first guidewire port on a distal end of the tubular housing of the circulatory support device, a second guidewire port on a sidewall of the tubular housing of the circulatory support device and distal to the impeller, and a third guidewire port on a proximal side of the impeller; the tubular body of the insertion tool has a length within a range of from about 85 mm to about 160 mm and an inside diameter within a range of from about 4.5 mm to about 6.5 mm; the tubular housing of the circulatory support device comprises an inlet tube coupled with a motor housing, the inlet tube having one or more distal pump inlets and one or more proximal pump outlets, and the impeller adjacent the one or more proximal pump outlets; the system does not require purging; the introducer sheath is a 16 French (Fr) sheath; the circulatory support device is configured to provide a flow rate of blood of about 4.0 liters per minute (l/min) for about 6 hours; the insertion tool comprises a hemostatic valve; the insertion tool comprises a locking mechanism, the locking mechanism comprising a recess configured to accept a locking pad configured to releasably lock with the circulatory support catheter; the insertion tool comprises a housing surrounding at least a portion of the locking mechanism, the housing comprising opposing first inner surface walls spaced farther than opposing second inner surface walls, wherein the at least a portion of the locking mechanism comprises radially outwardly extending tabs, and wherein the housing is configured to rotate to inwardly compress the tabs to prevent axial movement of the circulatory support catheter; inward compression of the tabs of the locking mechanism compresses the locking pad against the circulatory support catheter; the impeller is configured to be rotated by the motor via a shaft; the circulatory support device comprises an annular polymeric seal around the shaft; the circulatory support device comprises a seal around the shaft, the seal comprising a distal radial shaft seal having a distal side configured to face distally toward the impeller and a radially inner lip configured to contact the shaft and to extend from the distal side in a proximal direction toward the motor; further comprising a proximal radial shaft seal having a proximal side configured to face proximally toward the motor and a radially inner lip configured to contact the shaft and to extend from the proximal side in a distal direction toward the impeller; the impeller is configured to be rotated by the motor via a magnetic coupling; the introducer sheath comprises a hub on a proximal end of the introducer sheath, the hub having a feature for preventing axial and optionally rotational movement of the insertion tool; the hub and a relief bend disposed between the hub and the tubular body of the introducer sheath are configured to axially movably receive the tubular body of the insertion tool; the insertion tool comprises a tube with a valve in fluid communication with an inner lumen of the tubular body of the insertion tool configured for flushing with saline; a distal end of the tubular body of the insertion tool detachably connects to a guidewire aid configured to facilitate entry of a guidewire through the first guidewire port; a removable guidewire guide tube enters the first guidewire port on the distal end of the tubular housing, exits the tubular housing via the second guidewire port on the sidewall of the tubular housing distal to the impeller, reenters the tubular housing via the third guidewire port on the proximal side of the impeller, and extends proximally into the catheter shaft; the tubular body of the insertion tool is configured to receive the circulatory support device with the removable guidewire guide tube; the tubular body of the insertion tool and the guidewire guide tube are transparent; the insertion tool comprises a plug disposed at a proximal end of the insertion tool configured to connect to a sterile shield sleeve; a mechanical circulatory support system comprising an elongate flexible catheter shaft having a proximal end and a distal end, a circulatory support device carried by the distal end of the catheter shaft, the circulatory support device comprising a tubular housing, a motor, and an impeller configured to be rotated by the motor, wherein the circulatory support device is configured to provide a flow rate of blood of up to about 4.0 liters per minute (l/min) for about 6 hours without purging of the system; the system further comprises an insertion tool having a tubular body and configured to axially movably receive the circulatory support device; the system further comprises an introducer sheath having a tubular body and configured to axially movably receive the insertion tool; the system further comprises a controller that does not include a purging component; the controller does not include a cassette or a port for purging; the impeller comprises a blade having a proximal vane section with a wave-shaped vane curvature defined by one or more curved portions of a skeleton line of the blade; the tubular housing of the circulatory support device comprises an inlet tube with a main body, wherein the main body comprises a first attachment section at a first end of the main body configured to attach the inlet tube to a head unit of the circulatory support device and a second attachment section at a second end of the main body, wherein the first attachment section is configured to connect to the head unit in a form-locking and/or force-locking manner, wherein the main body further comprises a structural section comprising at least one stiffening recess between the first attachment section and the second attachment section; the impeller comprises a blade having at least one blade section having a wavy blade curvature; the tubular housing of the circulatory support device comprises an inlet tube having an inlet and an outlet, and wherein the outlet and the blade section having the wavy blade curvature at least partially axially overlap; the impeller comprises a blade element having a profile with camber lines, wherein a curvature of each of the camber lines when unwound into a plane increases along the axis of rotation in a direction starting from the pump intake section towards the outlet opening to an inflection point at which a blade angle (β) of the blade element is at a maximum, and wherein the curvature of each of the camber lines decreases after the inflection point, and wherein, in a region of the impeller located radially relative to an axis of rotation of the impeller and having a blade height SH of the blade element defined relative to a maximum blade height SHMAX such that 25%≤SH/SHMAX≤100%, the inflection point of each of the camber lines is located in a region of an upstream edge of an outlet opening of an inlet tube of the tubular housing; the system further comprises the tubular housing comprising an outlet opening configured to facilitate outflow of the blood and a diffuser configured to couple with the tubular housing, wherein, in an operating position, the diffuser is configured to guide the blood transversely to the outlet opening after the blood has passed through the outlet opening; the tubular housing comprises an inlet tube having a mesh section with a mesh structure formed from at least one mesh wire; the mesh section is bent at an obtuse angle at a bending point; the tubular housing comprises an inlet tube for conveying the blood through the inlet tube, and a reduced diameter section at a distal end of the inlet tube; the tubular housing comprises a feed head portion comprising at least one introduction opening for receiving the fluid flow into the feed line, and a contoured portion disposed adjacent to the feed head portion and comprising an inner surface contour, wherein the inner surface contour comprises a first inner diameter at a first position, a second inner diameter at a second position, and a third inner diameter at a third position, wherein the first inner diameter is greater than the second inner diameter, wherein the third inner diameter is greater than the second inner diameter, wherein the first inner diameter comprises a maximum inner diameter of the contoured portion and the second inner diameter comprises a minimum inner diameter of the contoured portion, wherein the inner surface contour comprises a rounded portion at the second position, wherein the contoured portion comprises a first inner radius at the first position and a second inner radius at the second position, wherein the second inner radius is at most one fifth smaller than the first inner radius, and wherein the second position is located between the third position and the first position; the tubular housing comprises a radiopaque marker at a distal end of the tubular housing; the tubular housing comprises an inlet tube with a nose piece at a distal end of the inlet tube, the nose piece comprising a radiopaque marker; the insertion tool comprises a hemostatic valve; the insertion tool comprises a locking mechanism, the locking mechanism comprising a recess configured to accept a locking pad configured to releasably lock with the catheter shaft; the insertion tool comprises a housing surrounding at least a portion of the locking mechanism, the housing comprising opposing first inner surface walls spaced farther than opposing second inner surface walls, wherein the at least a portion of the locking mechanism comprises radially outwardly extending tabs, and wherein the housing is configured to rotate to inwardly compress the tabs to prevent axial movement of the catheter shaft; inward compression of the tabs of the locking mechanism compresses the locking pad against the catheter shaft; a minimally invasive miniaturized percutaneous mechanical circulatory support system placed across the aortic valve via a single femoral arterial access point; the system may include a low profile axial rotary blood pump carried by the distal end of an eight French catheter; the system can be percutaneously inserted through the femoral artery and positioned across the aortic valve into the left ventricle; the device actively unloads the left ventricle by pumping blood from the left ventricle into the ascending aorta and systemic circulation; the impeller is configured to be rotated by the motor via a shaft; the circulatory support device comprises an annular polymeric seal around the shaft; the circulatory support device comprises a seal around the shaft, the seal comprising a distal radial shaft seal having a distal side configured to face distally toward the impeller and a radially inner lip configured to contact the shaft and to extend from the distal side in a proximal direction toward the motor; further comprising a proximal radial shaft seal having a proximal side configured to face proximally toward the motor and a radially inner lip configured to contact the shaft and to extend from the proximal side in a distal direction toward the impeller; the impeller is configured to be rotated by the motor via a magnetic coupling; the introducer sheath comprises a hub on a proximal end of the introducer sheath, the hub having a feature for preventing axial and optionally rotational movement of the insertion tool; the hub and a relief bend disposed between the hub and the tubular body of the introducer sheath are configured to axially movably receive the tubular body of the insertion tool; the insertion tool comprises a tube with a valve in fluid communication with an inner lumen of the tubular body of the insertion tool configured for flushing with saline; a distal end of the tubular body of the insertion tool detachably connects to a guidewire aid configured to facilitate entry of a guidewire through the first guidewire port; a removable guidewire guide tube enters the first guidewire port on the distal end of the tubular housing, exits the tubular housing via the second guidewire port on the sidewall of the tubular housing distal to the impeller, reenters the tubular housing via the third guidewire port on the proximal side of the impeller, and extends proximally into the catheter shaft; the tubular body of the insertion tool is configured to receive the circulatory support device with the removable guidewire guide tube; the tubular body of the insertion tool and the guidewire guide tube are transparent; the insertion tool comprises a plug disposed at a proximal end of the insertion tool configured to connect to a sterile shield sleeve; a mechanical circulatory support system for high risk coronary interventions including an elongate flexible catheter shaft having a proximal end and a distal end, a circulatory support device carried by the distal end of the shaft, the circulatory support device including a tubular housing, having a proximal end and a distal end, an impeller within the housing, a removable guidewire guide tube entering a first guidewire port on a distal end of the housing, exiting the housing via a second guidewire port on a side wall of the housing distal to the impeller, reentering the housing via a third guidewire port on a proximal side of the impeller, and extending proximally into the catheter shaft; the system may include a motor within the housing and configured to rotate the impeller; the motor may be positioned distal to the third guidewire port; the tubular housing may have an axial length in a range of 60 mm to 100 mm; the system may include a blood exit port on the tubular housing in communication with the impeller, and a blood intake port on the housing spaced distally apart from the blood exit port; the housing may include a flexible slotted tube covered by an outer polymeric sleeve; the system may include a sealed motor housing inside of the tubular housing; a mechanical circulatory support system for high risk coronary interventions including a circulatory support catheter, including a circulatory support device carried by an elongate flexible catheter shaft, an insertion tool having a tubular body and configured to axially movably receive the circulatory support device, and an access sheath (also referred to herein as an introducer sheath), having a tubular body and configured to axially movably receive the insertion tool; the access sheath may include an access sheath hub having an insertion tool lock for engaging the insertion tool; the access sheath hub may include a catheter shaft lock for locking the access sheath hub to the catheter shaft; the controller configured to drive a motor of a mechanical circulatory support system may be provided, wherein the controller does not include a purging component; the purging component can include a cassette or a port; the system does not require purging; a controller configured to drive a motor of a mechanical circulatory support system having a housing for mounting electronic components and a handle disposed on a top portion of the housing may be provided; the controller can include a visual alarm element wrapped around the handle on the top portion of the housing; the housing may not include more than one control element; the control element can be a rotary dial; the control element may be positioned on a first end of the housing; the controller may include a cable management system, said cable management system positioned on a second end opposite the first end; the controller may include a rotating securing attachment on a rear side of the housing; a minimally invasive miniaturized percutaneous mechanical left ventricular support system may be provided, optimized for treatment of patients experiencing cardiogenic shock; the system can include a low profile (e.g., 18 Fr to 19 Fr) ventricular support device (VSD) which includes an axial rotary blood pump and an elongate inlet tube, carried by the distal end of a nine French catheter; the system can be positioned to span the VSD across the aortic valve into the left ventricle, where it actively unloads the left ventricle by pumping blood from the left ventricle into the ascending aorta and systemic circulation, and may provide flow rates of up to about 6 L per minute at 60 mmHg; flow rates between 0.6 L per minute and 6 L per minute may be provided; intravascular access may be achieved using an 8 to 16 Fr (e.g., 8 to 10.5 Fr) introducer sheath, expandable to accommodate an 18 to 19 French VSD; access may be via percutaneous transfemoral puncture, or axillary access via a surgical cut down; the introducer sheath can be part of an introducer kit that may also include a guidewire, a dilator, an insertion tool, and a guidewire aid; the motor can be completely sealed by encapsulation within a motor housing, having a magnetic coupling to allow the motor to drive the impeller without the need for a shaft to leave the housing; the magnetic coupling can include a cylindrical driving magnet array positioned within the motor housing, concentrically positioned within a cylindrical driven magnet array located outside of the motor housing and mechanically coupled to the impeller; the impeller rotates with respect to the motor housing about a pivot jewel bearing; the magnetic coupling is flushed by a constant blood flow through flushing holes on proximal and distal ends of the magnetic coupling; the sealed motor enables elimination of a purging process necessary for certain competitive devices; migration of the device after placement may be inhibited by an intravascular anchor carried by the catheter shaft, which provides anchoring in the aorta; the anchor may include a plurality of radially outwardly expandable struts, carried by the catheter shaft, configured to contact the wall of the aorta and anchor the shaft against migration while allowing perfusion through the anchor struts; migration may be inhibited by a locking mechanism that engages the catheter shaft in a fixed position with an introducer sheath that is held to an arteriotomy with sutures, thus holding the catheter shaft still relative to the endovascular access pathway; onboard sensors can enable real time actual measurement of any of a variety of parameters of interest, such as aortic pressure, left ventricular pressure (including left ventricular end-diastolic pressure or “LVEDP”) temperature and blood flow velocity or others depending upon the desired clinical performance; sensors may be included on a distal end of the device, such as distal end of an inlet tube on a distal side of the blood outflow port; additional sensors may be provided on the proximal end of the elongate body, such as proximal to the blood outflow ports; specific sensors may include at least a first MEMS pressure and temperature sensor for direct measurement of absolute left ventricular pressure; sensors that enable extraction of important physiological parameters such as LVEDP; ultrasound transducers may be provided, for direct measurement of blood flow volume through the pump or optionally around the pump; ultrasound transducer surfaces may be curved and configured for increased focus and high sensitivity; a second MEMS pressure and temperature sensor may be provided on the proximal end of the inlet tube, such as to enable direct measurement of absolute aortic pressure and allow for differential pressure measurement; other forms of sensors may be used to assess flow rate such as laser doppler, thermal or electrical impedance sensors; flexible electrical conductors may extend along the length of the inlet tube for connecting distal and proximal sensors into an integrated system; the flexible conductors may be in the form of a flexible PCB, which can extend axially in a spiral around the inlet tube, in between the proximal and distal sensors; multi conductor cable bundles extend proximally through the elongate, flexible tubular body, to connectors at a proximal manifold, for releasable connection to an external electronic control unit; a mechanical ventricular support system for cardiogenic shock may include an elongate flexible catheter shaft, having a proximal end and a distal end, a ventricular support device carried by the distal end of the shaft, the ventricular support device including a ventricular support device housing, a motor, rotationally fixed with respect to a drive magnet array, an impeller, rotationally fixed with respect to a driven magnet array, and a sealed motor housing, inside of the ventricular support device housing, and encasing the motor and the drive magnet array; the system may include a removable guidewire guide tube; the guide tube may enter a first guidewire port on a distal end of the housing, exit the housing via a second guidewire port on a side wall of the housing distal to the impeller, reenter the housing via a third guidewire port on a proximal side of the impeller, and extend proximally into the catheter shaft; the system may include at least one inlet port and at least one outlet port on the housing separated by a flexible section of the housing; the distance between the inlet port and outlet port may be at least about 60 mm and no longer than 100 mm, preferably 70 mm; the system may include a first pressure sensor proximate the inlet port; the system may include a second pressure sensor on a proximal side of the outlet port; the system may include a visual indicium on the catheter shaft, within the range of from about 50 mm to about 150 mm from the distal end of the catheter shaft (or beginning of the pump); the motor may be positioned distal to the third guidewire port; the system may include an ultrasound transducer proximate the inlet port; the system may include a guidewire aid removably carried by the ventricular support device; the guidewire aid can include a tubular body having a distally facing opening and an inside diameter that increases in the distal direction to the opening; the guidewire aid may include a guidewire guide tube attached to the body; the guidewire guide tube can include a split line for splitting the guide tube so that the guide tube can be peeled away from a guidewire extending through the tube; the flexible section of the housing may include a flexible slotted tube covered by an outer polymeric sleeve; a mechanical ventricular support system for high risk coronary interventions may include a ventricular support catheter, including a ventricular support device carried by an elongate flexible catheter shaft, a sealed motor and an impeller inside the ventricular support device and rotationally coupled together by a magnetic bearing, an insertion tool having a tubular body and configured to axially movably receive the ventricular support device, and an access sheath, having a tubular body and configured to axially movably receive the insertion tool; the access sheath may include an access sheath hub having a first lock for engaging the insertion tool; the access sheath hub may include a second lock for engaging the catheter shaft; a controller configured to drive a motor of a mechanical circulatory support system, wherein the controller does not include a purging component; the purging component can include a cassette or a port; the system does not require purging; a controller configured to drive a motor of a mechanical circulatory support system having a housing for mounting electronic components and a handle disposed on a top portion of the housing may be provided; the controller can include a visual alarm element wrapped around the handle on the top portion of the housing; the housing may not include more than one control element; the control element can be a rotary dial; the control element may be positioned on a first end of the housing; the controller may include a cable management system, said cable management system positioned on a second end opposite the first end; the controller may include a rotating securing attachment on a rear side of the housing; a method of transcatheter delivery of a pump to the heart, the method comprising advancing the pump through vasculature, wherein the pump is advanced having a guidewire that extends through a first section of a catheter shaft located distal to the pump, through a tubular housing of the pump, external to an impeller and motor of the pump, and back into a second section of the catheter shaft located proximal to the pump; starting the motor and/or rotating the impeller prior to removal of the guidewire from the pump and/or prior to placement of the pump in the heart; and/or leaving the guidewire in the pump during use of the pump so the guidewire and/or pump at least partially remains in the left ventricle.

### BRIEF DESCRIPTION OF THE DRAWINGS

The foregoing and other features of the present disclosure will become more fully apparent from the following description and appended claims, taken in conjunction with the accompanying drawings. Understanding that these drawings depict only several embodiments in accordance with the disclosure and are not to be considered limiting of its scope, the disclosure will be described with additional specificity and detail through use of the accompanying drawings. In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here. It will be readily understood that the aspects of the present disclosure, as generally described herein, and illustrated in the drawings, can be arranged, substituted, combined, and designed in a wide variety of different configurations, all of which are explicitly contemplated and make part of this disclosure.

FIG. 1 is a cross sectional rendering of an embodiment of a mechanical circulatory support (MCS) device of the present disclosure carried by a catheter and positioned across an aortic valve via a femoral artery access.

FIG. 2 schematically illustrates an MCS system inserted into the body via the access pathway from the femoral artery to the left ventricle according to some embodiments.

FIG. 3 is a side elevational view of an embodiment of an MCS system that may incorporate the various features described herein.

FIG. 4 is the system of FIG. 3, with the introducer sheath removed and including an insertion tool and a guidewire loading aid.

FIG. 5 shows an introducer kit having a sheath and dilator, that may be used with the various MCS systems and methods described herein.

FIG. 6 shows an embodiment of a placement guidewire that may be used with the various MCS systems and methods described herein.

FIG. 7 is a partial perspective view of a distal, pump region of the MCS device.

FIGS. 8A and 8B are a side elevational view and close up detail view respectively of a distal region of the MCS device, showing the guidewire guide tube defining the guidewire path and the guidewire back loading aid in place.

FIGS. 9A and 9B are respectively a side view of a pump region of the MCS device and a cross sectional view through the impeller region of the MCS device.

FIG. 10A is a front elevational view of an MCS controller.

FIG. 10B is a rear perspective view of the MCS controller.

FIG. 11 illustrates a block diagram of an electronic system that can be housed inside the controller of FIGS. 10A and 10B.

FIG. 12 illustrates an exploded view with components of the electronic system of FIG. 11 inside the controller.

FIG. 13 illustrates a side perspective view of the MCS controller.

FIG. 14A illustrates a graph showing pressure difference between aortic pressure and left ventricular pressure.

FIG. 14B illustrates a graph showing applied current for a constant rotational speed of a motor shaft.

FIG. 15 illustrates an example user interface for displaying control parameters.

FIG. 16A illustrates an example user interface in a configuration mode.

FIG. 16B illustrates an example user interface in an operating mode.

FIGS. 17A and 17B illustrate embodiments of an electronic control element.

FIGS. 18A to 18D are example left ventricle (LV) pressure curves illustrating a process for determining left ventricular end-diastolic pressure (LVEDP).

FIG. 19 is a side view of an alternative embodiment of a pump of an MCS system.

FIGS. 20A-20B are side views of an impeller and a partial side view of an impeller blade, respectively, illustrating an embodiment of an impeller of an MCS system.

FIG. 21A-21C illustrate embodiments of a pump region of an MCS system.

FIG. 22 is a side view of an embodiment of an inlet tube of an MCS system.

FIG. 23 is a perspective view of an embodiment of an inlet tube of an MCS system.

FIG. 24 is a perspective view of an embodiment of a pump region of an MCS system.

FIG. 25 is a partial cross sectional view of a contour section of an inlet tube of the pump region of FIG. 24.

FIGS. 26A-26E are various views of an embodiment of an insertion tool that may be used with the various MCS systems described herein.

FIG. 27 is a partial cross sectional view, through an impeller and magnetic coupling region, of an embodiment of a pump that may be used with the various MCS systems described herein.

FIGS. 28A and 28B are side and perspective views respectively of an ultrasound transducer that may be used with the various MCS systems described herein.

FIG. 29 is a side elevational view of an introducer sheath and hub that may be used with the various MCS systems described herein.

FIGS. 30A-30C are various views of another embodiment of an MCS device having two lip seals facing one another.

While the above-identified drawings set forth presently disclosed embodiments, other embodiments are also contemplated, as noted in the detailed description. This disclosure presents illustrative embodiments by way of representation and not limitation. Numerous other modifications and embodiments can be devised by those skilled in the art which fall within the scope and spirit of the principles of the presently disclosed embodiments.

### DETAILED DESCRIPTION

The following detailed description is directed to certain specific embodiments of the development. In this description, reference is made to the drawings wherein like parts or steps may be designated with like numerals throughout for clarity. Reference in this specification to “one embodiment,” “an embodiment,” or “in some embodiments” means that a particular feature, structure, or characteristic described in connection with the embodiment is included in at least one embodiment of the invention. The appearances of the phrases “one embodiment,” “an embodiment,” or “in some embodiments” in various places in the specification are not necessarily all referring to the same embodiment, nor are separate or alternative embodiments necessarily mutually exclusive of other embodiments. Moreover, various features are described which may be exhibited by some embodiments and not by others. Similarly, various requirements are described which may be requirements for some embodiments but may not be requirements for other embodiments. Reference will now be made in detail to embodiments of the invention, examples of which are illustrated in the accompanying drawings. Wherever possible, the same reference numbers will be used throughout the drawings to refer to the same or like parts.

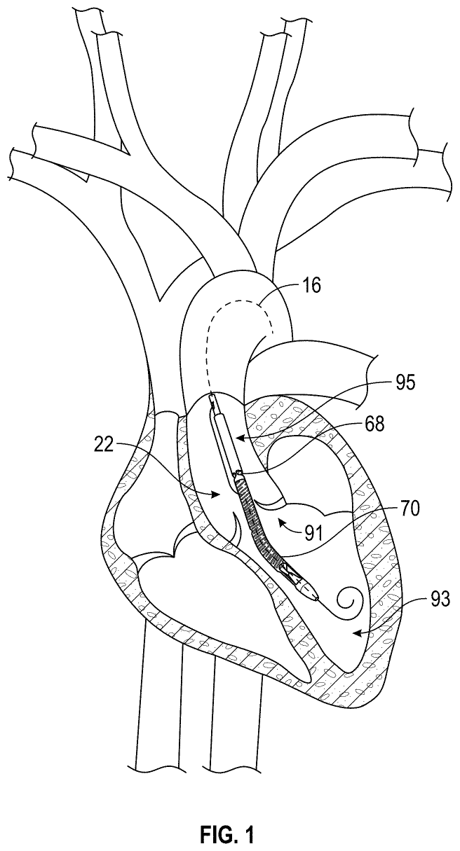

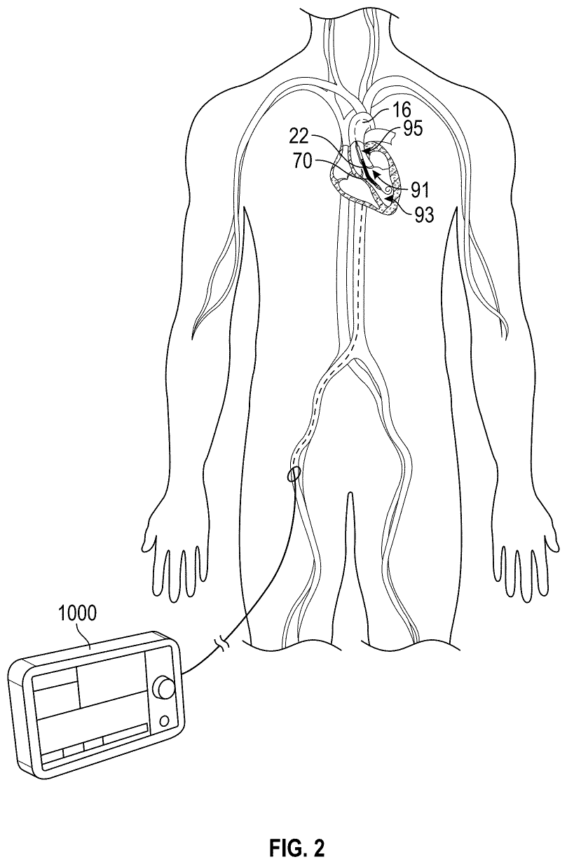

FIG. 1 is a schematic of a distal end of an embodiment of a mechanical circulatory support (MCS) system 10 having a pump 22 mounted on the tip of a catheter 16 placed in the heart. FIG. 2 schematically illustrates an MCS system inserted into the body via the access pathway from the femoral artery to the left ventricle according to some embodiments. Some features of the MCS system 10 will be described with respect to FIGS. 1 and 2, with further detail of various features provided elsewhere herein.

Various embodiments of the MCS system 10 are described herein having various features. In some embodiments, the MCS system 10 may include a temporary (e.g., generally no more than about 6 hours, or in some embodiments no more than about 3 hours, no more than about 4 hours, no more than about 7 hours, no more than about 8 hours, no more than about 9 hours, or no more than about 10 hours) left ventricular support device or pump, also referred to as an MCS pump or MCS device. The device may be used during high-risk percutaneous coronary intervention (PCI) performed in elective or urgent, hemodynamically stable patients with severe coronary artery disease and/or depressed left ventricular ejection fraction, e.g. when a heart team, including a cardiac surgeon, has determined high risk PCI is the appropriate therapeutic option. The pump is placed across the aortic valve via a single femoral arterial access.

In some embodiments, the MCS system 10 may include a longer-term pump 22, for example as therapy for cardiogenic shock. The MSC system 10 may include the pump 22 having a first magnet rotated by a motor within a sealed motor housing. An impeller with a second magnet may partially surround the first magnet external to the motor housing. Rotation of the first magnet causes, via magnetic communication, the second magnet and impeller to rotate.

In some embodiments, the MCS system 10 may include an insertion tool having a tubular body and configured to axially movably receive the circulatory support device. An introducer sheath, having a tubular body, may be configured to axially movably receive the insertion tool. The insertion tool may protect the circulatory support device, for example during insertion in the sheath.

In some embodiments, the MCS system 10 may include a low-profile axial rotary blood pump mounted on the catheter 16, such as an 8 French (Fr) catheter. When in place, the MCS pump 22 may be driven by an MCS controller to provide up to about 4.0 liters/minute of partial left ventricular support, which may be at about 60 mm Hg. No system purging is needed due to improved bearing design and sealed motor. The MCS system 10 or portions thereof may be visualized fluoroscopically, eliminating the need for placement using sensors.

In some embodiments, the MCS system 10 may include an introducer sheath. The sheath may be expandable. The expandable sheath may allow for example an 8 to 10 Fr initial access size for easy insertion and closing, expandable to allow introduction of 14 Fr, 16 Fr, and 18 Fr pump devices, and return to a narrower diameter around the 8 Fr catheter once the pump has passed. This feature may allow passage of the pump 22 through vasculature while minimizing shear force within the blood vessel, advantageously reducing risk of bleeding and healing complications. Distention or stretching of an arteriotomy may be done with radial stretching with minimal shear, which is less harmful to the vessel. Access may be accomplished via transfemoral, transaxillary, transaortal, or transapical approach. In some embodiments, an expandable sheath may allow 8 to 16 Fr (e.g., 8 to 10.5) Fr initial access size for easy insertion and closing, expandable to allow introduction of at least about a 14 Fr, a 16 Fr, an 18 or 19 Fr device.

In some embodiments, an inlet tube 70 of the pump 22 extends across the aortic valve 91. An impeller may be located at the outflow section 68 (also referred to as a pump outlet herein) of the inlet tube 70, drawing blood from the left ventricle 93 through the inlet tube 70 and ejecting it out the outflow section 68 into the ascending aorta 95. The motor may be mounted directly proximal to the impeller in a sealed housing, eliminating the need to purge or flush the motor prior to or during use. This configuration provides hemodynamic support during high-risk PCI, with sufficient time and safety for a complete revascularization via a minimally invasive approach (rather than an open surgical procedure).

In some embodiments, the MCS system 10 actively unloads the left ventricle by pumping blood from the ventricle into the ascending aorta and systemic circulation. When in place, the MCS device may be driven by the complementary MCS Controller to provide between 0.4 l/min up to 4.0 l/min of partial left ventricular support. The MCS system 10 may eliminate the need for motor flushing, provide increased flow performance up to 4.0 l/min at 60 mmHg with acceptably safe hemolysis due to a computational fluid dynamics (CFD) optimized impeller that minimizes shear stress. When in place, the VSD can be driven by the complementary ventricular support controller 1000 to provide between 0.4 l/min up to 6.0 l/min of partial left ventricular support. In some embodiments, the VSD can be driven by the complementary ventricular support controller 1000 to provide between 0.6 l/min up to 6.0 l/min of partial left ventricular support. A range between 0.6 l/min up to 6.0 l/min may allow for 10 equidistant flow levels, for example.

In some embodiments, the MCS system 10 may include an 18 to 19 Fr axial rotary blood pump and inlet tube assembly mounted on the catheter 16, such as a catheter no larger than 10.5 Fr. When in place, the ventricular support pump 22 can be driven by a ventricular support controller 1000, which may provide at least about 4 or 5 and up to about 6.0 liters/minute of partial left ventricular support, at about 60 mm Hg pressure differential. In some embodiments of the pump 22, no system purging is needed due to the encapsulated motor and magnetic bearing design.

In general, the overall MCS system 10 may include a series of related subsystems and accessories, including one or more of the following: The MCS system 10 may include a pump, shaft, proximal hub, insertion tool, proximal cable, infection shield, guidewire guide tube and/or guidewire aid. The pump 22 may be provided sterile. An MCS shaft may contain the electrical cables and a guidewire lumen for over-the-wire insertion. The proximal hub contains guidewire outlet with a valve to maintain hemostasis and connects the MCS shaft to the proximal cable, that connects the pump 22 to the controller 1000. The proximal cable 28 may be 3.5 m (approx. 177 inch) in length and extend from a sterile field 5 to a non-sterile field 3 where the controller 1000 is located. An MCS insertion tool may be provided pre-mounted on the MCS Device to facilitate the insertion of the pump into the introducer sheath and to protect the inlet tube and the valves from potential damage or interference when passing through the introducer sheath. A peel-away guidewire aid may be pre-mounted on the MCS Device to facilitate the insertion of a guidewire, such as an 0.018″ placement guidewire, into the pump 22 and into the MCS catheter shaft 16, optionally with the MCS insertion tool also pre-mounted such that the guidewire guide tube may pass at least in part through a space between the MCS Device and the MCS insertion tool. A 3 m, 0.018″ placement guidewire may be used, having a soft coiled pre-shaped tip for atraumatic wire placement into the left ventricle. The guidewire may be provided sterile. A 14 Fr or 16 Fr introducer sheath may be used with a usable length of 275 mm to maintain access into the femoral artery and provide hemostasis for a 0.035″ guidewire, a diagnostic catheter, the 0.018″ placement guidewire, and the insertion tool. The housing of the introducer sheath may be designed to accommodate the MCS insertion tool. The introducer sheath is provided sterile. An introducer dilator may be compatible with the introducer sheath to facilitate atraumatic insertion of the introducer sheath into the femoral artery. The introducer dilator is provided sterile. The controller 1000 may be used which drives and operates the pump 22, observes its performance and condition, and/or provides error and status information. The powered controller 1000 may be designed to support at least about 12 hours of continuous operation and contains a basic interface to indicate and adjust the level of support provided to the patient. Moreover, the controller 1000 may provide an optical and audible alarm notification in case the system detects an error during operation. The controller 1000 may be provided non-sterile and be contained in an enclosure designed for cleaning and re-use outside of the sterile field 5. The controller 1000 enclosure may contain a socket into which the extension cable is plugged.

In some embodiments, the pump 22 (which may also be referred to as a ventricular support device (VSD) or mechanical circulatory support device) of the present disclosure may be a temporary (generally no more than about 6 days) left ventricle support device for enhancing cardiac output in cardiogenic shock patients such as caused by acute ST elevation myocardial infarction. The pump 22 may be placed across the aortic valve, typically via transvascular access, to pump blood from the left ventricle to the ascending aorta.

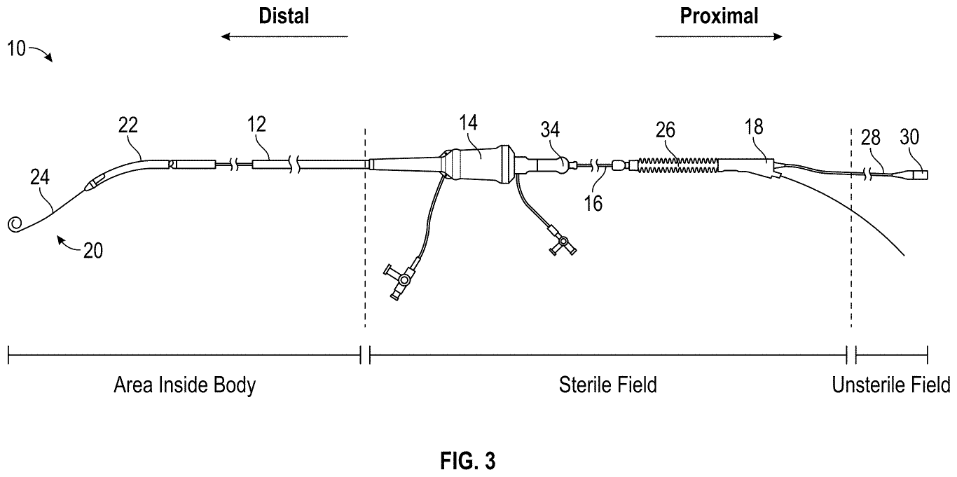

Referring to FIG. 3, there is illustrated an overall MCS system 10 in accordance with some embodiments, subcomponents of which will be described in greater detail below. For reference, the “distal” and “proximal” directions are indicated by arrows in FIGS. 3, 4 and 8A. “Distal” and “proximal” as used herein have their usual and customary meaning, and include, without limitation, a direction more distant from an entry point of the patient's body as measured along the delivery path, and away a direction less distant from an entry point of the patient's body as measured along the delivery path, respectively.

The system 10 may include an introducer sheath 12 having a proximal introducer hub 14 with a central lumen for axially movably receiving an MCS shaft 16 (the MCS shaft may also be referred to as a catheter, catheter shaft, and/or a shaft herein). The MCS shaft 16 may extend between a proximal hub 18 and a distal end 20 of the system 10, with a guidewire 24 extending therefrom. The guidewire 24 or any other guidewire described herein may have various features, such as those described in U.S. Provisional Application No. 63/224,326, titled GUIDEWIRE and filed Jul. 21, 2021, the entire content of which is incorporated by reference herein for all purposes and forms a part of this specification. The hub 18 may be provided with an integrated Microcontroller or memory storage device for device identification and tracking of the running time, which could be used to prevent overuse to avoid excessive wear or other technical malfunction. The microcontroller or memory device could disable the device, for example to prevent using a used device. They could communicate with the controller, which could display information about the device or messages about its usage. An atraumatic cannula tip with radiopaque material allows the implantation/explanation to be visible under fluoroscopy.

The pump 22 comprises a tubular housing. The tubular housing of the pump 22 is used broadly herein and may include any component of the pump 22 or component in the pump region of the system, such as an inlet tube, a distal endpiece, a motor housing, other connecting tubular structures, and/or a proximal back end of the motor housing. The pump 22, for example the tubular housing, is carried by a distal region of the MCS shaft 16. The system 10 is provided with at least one central lumen for axially movably receiving the guidewire 24. The proximal hub 18 is additionally provided with an infection shield 26. A proximal cable 28 extends between the proximal hub 18 and a connector 30 for releasable connection to a control system typically outside of the sterile field 3, to drive the pump 22.

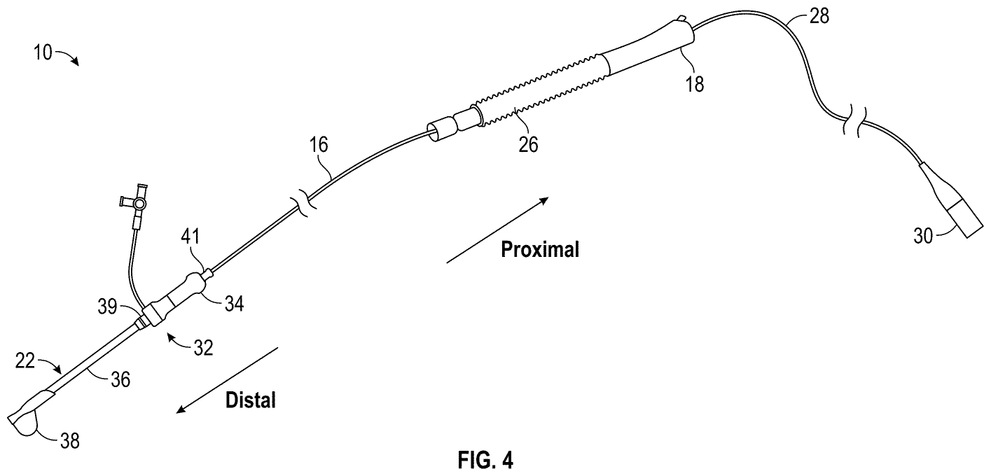

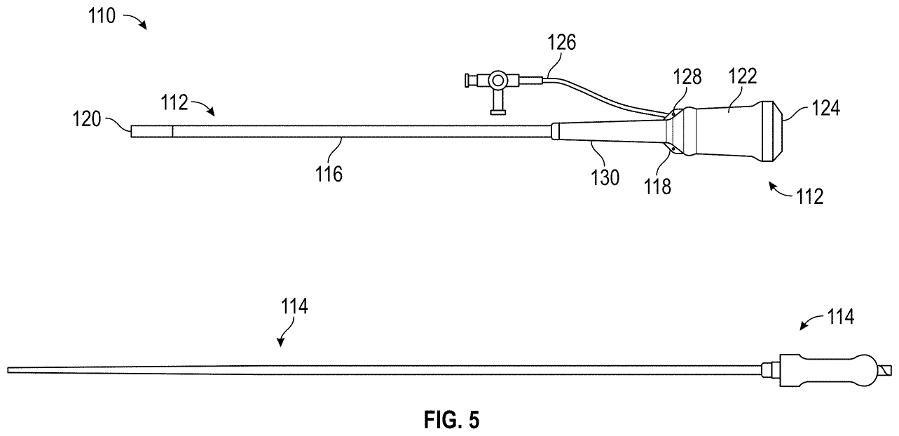

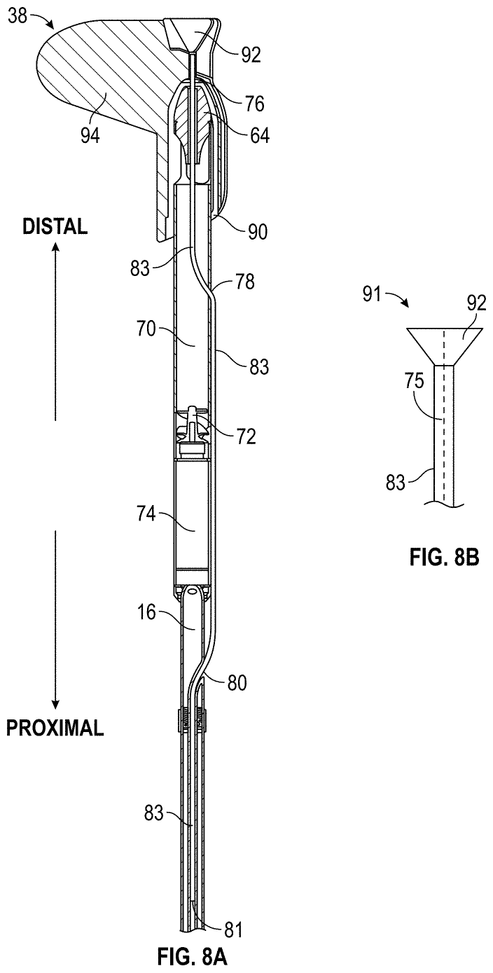

Referring to FIG. 4, the system 10 may additionally include an insertion tool 32, having an elongate tubular body 36 having a length within the range of from about 85 mm to about 160 mm (e.g., about 114 mm) which may be adapted to span the length of the hub 122 and bend relief 130 of the introducer sheath 112 (see FIG. 5) and an inside diameter within the range of from about 4.5 mm to about 8.0 mm (e.g., about 5.55 mm), extending distally from a proximal hub 34. The tubular body 36 includes a central lumen adapted to axially movably receive the MCS shaft 16 and pump 22 there through, and sufficient collapse resistance to maintain patency when passed through the hemostatic valves of the introducer sheath. As illustrated in FIG. 4, the pump 22 can be positioned within the tubular body 36, such as to facilitate passage of the pump 22 through the hemostatic valve(s) on the proximal end of an introducer hub 14. A marker 37 (FIG. 7) is provided on the MCS shaft 16 spaced proximally from the distal tip 64 such that as long as the marker 37 is visible on the proximal side of the hub 34, the clinician knows that the pump is within the tubular body 36.

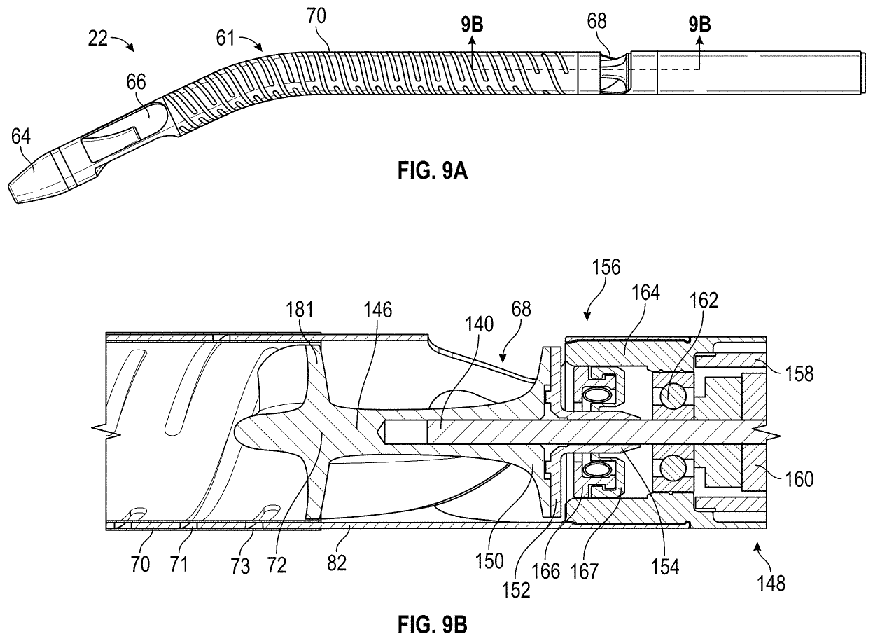

The hub 34 may be provided with a first engagement structure 39 for engaging a complimentary second engagement structure on the introducer sheath to lock the insertion tool into the introducer sheath. The hub 34 may be connected with the infection shield 26 via a connection 41, such as a knob or button that connects via force-fit, screw, or other means. The hub 34 may also be provided with a locking mechanism for clamping onto the shaft 16 to prevent the shaft 16 from sliding proximally or distally through the insertion tool once the MCS device has been positioned at the desired location in the heart. The locking mechanism may be actuated by twisting one or more parts (for example, two parts) of the hub 34. Other actuation means may also be possible. The hub 34 may additionally be provided with a hemostasis valve to seal around the shaft 16. In some embodiments, the hub 34 may accommodate passage of the larger diameter MCS device which includes the pump. In one commercial presentation of the system, the MCS device as packaged is pre-positioned within the insertion tool and the guidewire aid is pre-loaded within the MCS device and shaft 16, as illustrated in FIG. 4. In some examples, the MCS device is configured to be prepositioned in the tube 36 and advanced distally. In such a configuration, the lumen in the hub 34 may be smaller than the MCS device and only the shaft 16 may be configured to pass through the hub 34. When removing the pump from the body, the MCS device may be pulled into the tube 36 and then the insertion tool may be pulled out of the introducer with the pump in the tube 36. Further details of a guidewire aid 38 are discussed, for example, with reference to FIGS. 8A and 8B.

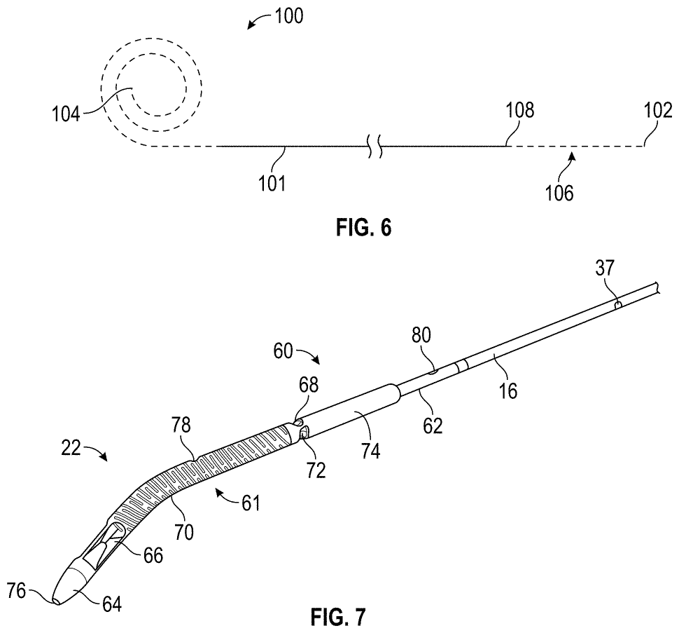

Referring to FIGS. 5 and 6, an introducer kit 110 may include a guidewire 100, an introducer sheath 112, a dilator 114, and/or a guidewire aid 38, such as discussed with reference to FIGS. 8A and 8B. The guidewire 100 and introducer sheath 112 may correspond to guidewire 24 and introducer sheath 12 discussed above. The guidewire 100 (e.g., 0.018″ placement guidewire) may comprise an elongate flexible body 101 extending between a proximal end 102 and a distal end 104. A distal zone of the body 101 may be pre-shaped into a J tip or a pigtail, as illustrated in FIG. 6, to provide an atraumatic distal tip. A proximal zone 106 may be configured to facilitate threading through the MCS device and may extend between the proximal end 102 and a transition 108. The proximal zone 106 may have an axial length within the range of from about 100 mm to about 500 mm (e.g., about 300 mm).

The introducer kit 110 may comprise the introducer sheath 112 and/or the dilator 114. The introducer sheath 112 may comprise an elongate tubular body 116, extending between a proximal end 118 and a distal end 120. The tubular body 116 terminates proximally in a proximal hub 122. Optionally, the tubular body 116 is expandable or can be peeled apart. The proximal hub 122 includes a proximal end port 124 in communication with a central lumen extending throughout the length of the tubular body 116 and out through a distal opening, configured for axially removably receiving the elongate dilator 114. Proximal hub 122 may additionally be provided with a side port 126, at least one and optionally two or more attachment features such as an eye 128 to facilitate suturing to the patient, and at least one and optionally a plurality of hemostasis valves for providing a seal around a variety of introduced components such as a standard 0.035″ guidewire, a 5 Fr or 6 Fr diagnostic catheter, an 0.018″ placement guidewire 100, the shaft 16, and the insertion tool 32. Proximal hub 122 may have a lock for preventing axial movement of the insertion tool 32 and/or the dilator 114.

FIG. 7 illustrates additional details of a distal pump region 60 of the MCS system showing the device or pump 22 and a distal portion of the catheter shaft 62. The pump zone or region 60 extends between a bend relief 62 at the distal end of shaft 16 and a distal tip 64. The pump 22 include a tubular housing 61, which may include an inlet tube 70, a distal tip 64, and/or a motor housing 74. The tubular housing 61 may include one or more pump inlets 66 and/or outlets 68, which may be part of the inlet tube 70, or part of other structures such as an intermediate structure joining a proximal end of the inlet tube 70 to the motor housing 74. A guidewire guide aid, as further described herein, may extend into and out of various components of the system, such as the tubular housing 61 of the pump 22 and/or the catheter shaft 16 (e.g., bend relief 62).

The pump inlet 66 comprises one or more windows or openings in fluid communication with a pump outlet 68 (also referred to as an outflow section herein) by way of a flow path extending axially through the inlet tube 70. The pump inlet may be positioned at about the transition between the inlet tube and the proximal end of distal tip 64, and in any event is generally within about 5 cm or 3 cm or less from the distal port 76.

In some embodiments, the distal tip 64 is radiopaque. For example, the distal tip may be made from a polymer containing a radiopacifier such as barium sulfate, bismuth, tungsten, iodine. In some embodiments, an entirety of the MCS device is radiopaque. In some embodiments, a radiopaque marker is positioned on the inlet tube 70 between the pump outlet 68 and the guidewire port 78 to indicate the current position of the MCS device relative to the aortic valve 91.

The inlet tube 70 may comprise a highly flexible slotted (e.g., laser cut) metal (e.g., Nitinol) tube having a polymeric (e.g., Polyurethane) tubular layer to isolate the flow path. Inlet tube 70 may have an axial length within the range of from about 60 mm to about 100 mm and in one implementation may be about 67.5 mm. The outside diameter of the inlet tube 70 may typically be within the range of from about 4 mm to about 5.4 mm, and in one implementation may be about 4.66 mm. The wall thickness of the inlet tube 70 may be within the range of from about 0.05 mm to about 0.15 mm. The connections between the inlet tube 70 and the distal tip 76 and to the motor may be secured such as through the use of laser welding, adhesives, threaded or other interference fit engagement structures, or may be via press fit.

The impeller 72 may be positioned in the flow path between the pump inlet 66 and pump outlet 68. In the illustrated embodiment, the impeller 72 is positioned adjacent to the pump outlet 68. As is discussed further below, the impeller 72 may be rotationally driven by a motor contained within motor housing 74, on the proximal side of the impeller 72.

FIGS. 8A and 8B are a side cross-sectional view and a detail view respectively of the pump region showing an embodiment of a guidewire aid 38. The MCS device can be provided in either a rapid exchange or over the wire configuration. A first guidewire port 76 such as a distal-facing opening on distal face of the distal tip 64 may be in communication, via a first guidewire lumen through the distal tip 64 and at least a portion of the flow path in the inlet tube 70, with a second guidewire port 78 such as an opening extending through a side wall of the inlet tube 70, and distal to the impeller 72. This could be used for rapid exchange, with the guidewire 100 extending proximally alongside the catheter from the second guidewire port 78.

The catheter may be provided in an over the wire configuration, in which the guidewire extends proximally throughout the length of the catheter shaft 16 through a guidewire lumen therein. In the over the wire embodiment of FIGS. 7, 8A and 8B, however, the guidewire 100 exits the inlet tube 70 via second guidewire port 78, extends proximally across the outside of the impeller and motor housing, and reenters the catheter shaft 16 via a third guidewire port 80, which may be an opening in the sidewall of the catheter shaft 16 or of a proximal component of the pump, motor housing, or backend. The third guide wire port 80 may be located proximal to the motor, and, in the illustrated embodiment, is located on the bend relief 62. Third guide wire port 80 is in communication with a guide wire lumen which extends proximally throughout the length of the shaft 16 and exits at a proximal guidewire port carried by or located within the proximal hub 18 (see FIG. 4).

As shown in FIG. 8A, the pump may be provided assembled with the removable guidewire aid 38. The guidewire aid 38 may have a guidewire guide tube 83. The guide tube 83 may be a cylindrical or other closed cross-sectional shape extending axially. The guide tube 83 may be a flexible, transparent material such as polyimide. The guide tube 83 may be adapted to be peeled apart longitudinally, such as having a longitudinal slit or tear line. The inside surface of the guide tube 83 may be provided with a lubricious coating, such as PTFE. The guide tube 83 may track the intended path of the guidewire 100 from the first guidewire port 76, proximally through the tip 64 and back outside of the inlet tube via second guidewire port 78, and back into the catheter shaft 16 via the third guidewire port 80. In the illustrated implementation, the guidewire guide tube 83 extends proximally within the catheter shaft 16 to a proximal end 81 of the guide tube 83, in communication with or within the guidewire lumen which extends to the proximal hub 18. The proximal end 81 of the guide tube 83 may be positioned within about 5 mm or 10 mm of the distal end of the shaft 16, or may extend into the catheter shaft guidewire lumen for at least about 10 mm or 20 mm, such as within the range of from about 10 mm to about 50 mm. In some embodiments, the third port 80 may be located within a proximal end of the tubular housing, such as the motor housing or backend, or in any other components of the device at a location that is proximal to the impeller.

The guidewire aid 38 may have a funnel 92. The funnel 92 may be located at a distal end of the guide tube 83 and be provided pre-positioned at a distal end of the inlet tube, for example at the distal tip 64. The funnel 92 may increase in width in the distal direction, from a narrow proximal end in communication with the guide tube 83, to a wider distal opening at a distal end of the funnel 92. The funnel 92 may be conical, frustoconical, pyramidal, segmented, or other shapes. A proximal end of the funnel 92 may be attached to a distal end of the guidewire guide tube 83. The proximal end 102 of the guidewire 100 (see FIG. 6) may be inserted into the funnel 92, passing through the first (distal) guidewire port 76 and guided along the intended path by tracking inside of the guidewire guide tube 83. The guidewire guide tube 83 may then be removed by sliding the guide tube 83 distally out of the distal tip 64 and peeling it apart longitudinally, leaving the guidewire 100 in place.

The guidewire aid 38 may have a pull tab 94. In some embodiments, a distal end of the guidewire guide tube 83 is attached to the pull tab 94 of the guidewire aid 38. The pull tab 94 may be a structure capable of being gripped by a human hand, for example with a lateral, planar extension as shown. The guidewire aid 38, for example, the pull tab 94, the guide tube 83 and/or the funnel 92, may be provided with a tearable line 75, as more clearly seen in FIG. 8B. The tearable line 75 line may be an axially extending split line. The tearable line 75 may include a weakening, a slot, or a perforated linear region. Removal of the guidewire aid 38 may be accomplished such as by grasping the pull tab 94 and pulling out the guidewire tube 83 and/or funnel 92 and removing them from the guidewire 100 as they split or peel away along the split line 75, such as shown in the detailed inset 91 of FIG. 8B.

The guidewire aid 38 may include a proximal opening 90 configured to slip over and removably receive the distal tip 64 and/or struts at the distal end of the inlet tube 70 that define windows of the pump inlet 66. The guidewire guide tube 83 having a lumen therethrough is positioned within the proximal opening 90 and aligned to pass through the guidewire port 76 of the distal tip 64. The proximal opening 90 may further be configured to slip over and removably receive a distal end of tubular body 36 of an insertion tool 32 as shown in FIG. 4. The MCS system may be dimensioned so that an annular space defined between the outer surface of the MCS device—such as the inlet tube 70, motor housing 74, or MCS catheter bend relief 16—and the inner surface of the tubular body 36 of the insertion tool 32, may removably receive the guidewire guide tube 83 therein, when the MCS device, guidewire aid 38 and insertion tool 32 are assembled together.

In some embodiments, the lumen of the guidewire guide tube 83 is in communication with the distal flared opening of the funnel 92 which gets larger in cross-section in the distal direction. The guidewire aid 38 may be provided assembled on the MCS pump with the guidewire guide tube 83 pre-loaded along a guidewire path, for example into the MCS pump through port 76, through a portion of the fluid path within the inlet tube 70, out of the MCS pump through port 78, along the exterior of the MCS pump and back into the shaft 16 through port 80. This helps a user guide the proximal end of a guidewire into the funnel 92 through the guidewire path and into the guidewire lumen of the MCS shaft 16. The pull tab 94 may be provided on the guidewire aid 38 to facilitate grasping and removing the guidewire aid, including the guidewire guide tube 83, following loading of the guidewire. The guidewire aid 38 may have a longitudinal slit or tear line 75, for example along the funnel 92, proximal opening 90 and guidewire guide tube 83, to facilitate removal of the guidewire aid 38 from the MCS pump 22 and guidewire 100

The guidewire aid 38 features described herein may be used with a variety of different MCS systems and/or pump devices. The guidewire aid 38 may be used for guidewire paths that enter and exit a pump housing, as described, or that do not exit a housing. The guidewire aid 38 is described herein as being used with an MCS system configured for temporary operation for high-risk PCI procedures. The system may include rotating impeller with a radial shaft seal and a motor rotating the impeller via shaft extending through the seal. The guidewire aid 38 may be used with a variety of different devices. The guidewire aid 38 may also be used with a pump having a magnetic drive, where the motor rotates a first magnet within a sealed motor housing that magnetically communicates with a second magnet of the impeller that is external to the sealed housing to rotate the impeller. Thus, the guidewire aid 38 is not limited to use with only the particular pump embodiments described herein.

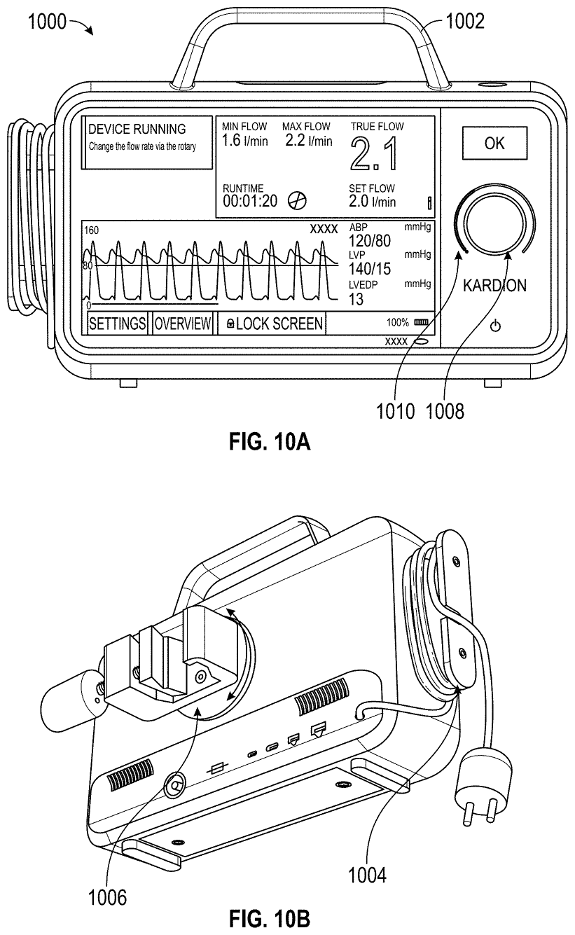

FIGS. 9A and 9B depict side views and a partial cross-section view respectively of the pump 22. As shown, the impeller 72 may be attached to a short, rigid motor drive shaft 140. In the illustrated implementation, the drive shaft 140 extends distally into a proximally facing central lumen in the impeller 72, such as through a proximal extension 154 on the impeller hub 146, where it may be secured by a press fit, laser weld, adhesives or other bonding technique. The impeller 72 may include a radially outwardly extending helical blade 181, which, at its maximum outside diameter, may be spaced apart from the inside surface of tubular impeller housing 82 within the range of from about 40 μm to about 120 μm. Impeller housing 82 may be a proximal extension of the inlet tube 70, on the proximal side of the slots 71 formed in the inlet tube 70 to provide flexibility distal to the impeller. A tubular outer membrane 73 may enclose the inlet tube 70 and seal the slots 71 while preserving flexibility of the inlet tube. Pump outlets 68 may be formed in the sidewall of the impeller housing 82, axially aligned for example with a proximal portion of the impeller 72 (e.g., a proximal 25% to 50% portion of the impeller).

The impeller 72 may comprise a medical grade titanium. This enables a CFD optimized impeller design with minimized shear stress for reduced damage of the blood cells (hemolysis) and a non-constant slope increasing the efficiency. This latter feature cannot be accomplished with a mold-based production method. Electro polishing of the surface of the impeller 72 may decrease the surface roughness to minimize the impact on hemolysis.

In some implementations, the impeller hub 146 flares radially outwardly in a proximal direction to form an impeller base 150, which may direct blood flow out of the outlets 68. A proximal surface of the impeller base 150 may be secured to an impeller back 152, which may be in the form of a radially outwardly extending flange, secured to the motor shaft 140. For this purpose, the impeller back 152 may be provided with a central aperture to receive the motor drive shaft 140 and may be integrally formed with or bonded to a tubular sleeve/proximal extension 154 adapted to be bonded to the motor drive shaft 140. In some implementations, the impeller back 152 is first attached to the motor drive shaft 140 and bonded such as through the use of an adhesive. In a second step, the impeller 72 may be advanced over the shaft and the impeller base 150 bonded to the impeller back 152 such as by laser welding.

The distal opening in the aperture in impeller back 152 may increase in diameter in a distal direction, to facilitate application of an adhesive. The proximal end of tubular sleeve/proximal extension 154 may decrease in outer diameter in a proximal direction to form an entrance ramp for facilitating advancing the sleeve proximally over the motor shaft and through a seal 156, discussed further below.

The motor 148 may include a stator 158 having conductive windings surrounding a cavity which encloses motor armature 160 which may include a plurality of magnets rotationally secured with respect to motor drive shaft 140. The motor drive shaft 140 may extend from the motor 148 through a rotational bearing 162 and also through the seal 156 before exiting the sealed motor housing 164 (also referred to herein as motor housing 74). Seal 156 may include a seal holder 166 which supports an annular seal 167 such as a polymeric seal ring. The seal ring includes a central aperture for receiving the tubular sleeve/proximal extension 154 and is biased radially inwardly against the tubular sleeve/proximal extension 154 to maintain the seal ring in sliding sealing contact with the rotatable tubular sleeve/proximal extension 154. The outside surface of the tubular sleeve/proximal extension 154 may be provided with a smooth surface such as by electro polishing, to minimize wear on the seal.

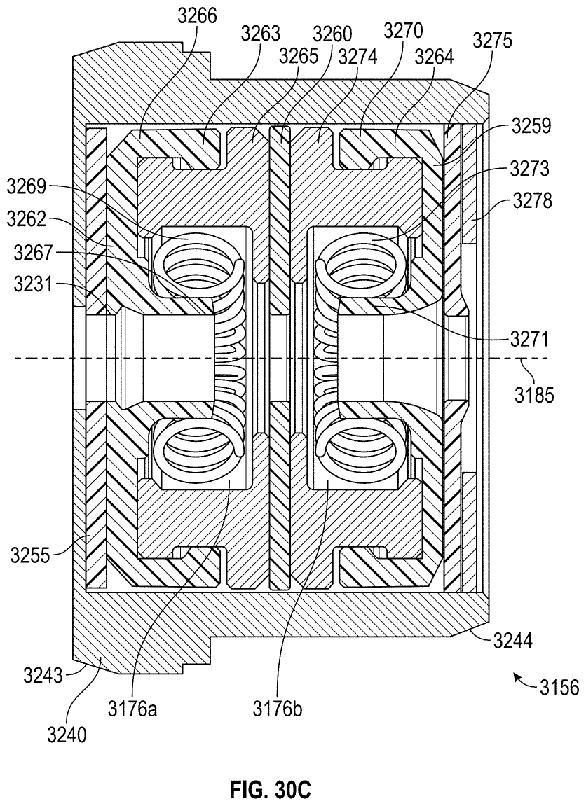

In some embodiments, the pump 22 may include a seal, and/or one or more features of a seal, as described herein with respect to FIGS. 30A-30C.

The pump may include a sealed motor, in applications with a short time of usage for high risk PCI (typically no more than about 6 hours), and be configured for use without flushing or purging. This provides the opportunity to directly bond the impeller 72 on the motor drive shaft 140 as discussed in further detail below, removing issues sometimes associated with magnetic coupling such as the additional stiff length, space requirements or pump efficiency. A four pole motor design enables flow performance up to 4.0 lmin−1 (liters per minute) at 60 mmHg with low temperature change. The motor cable interface may be provided with a high tensile strength.

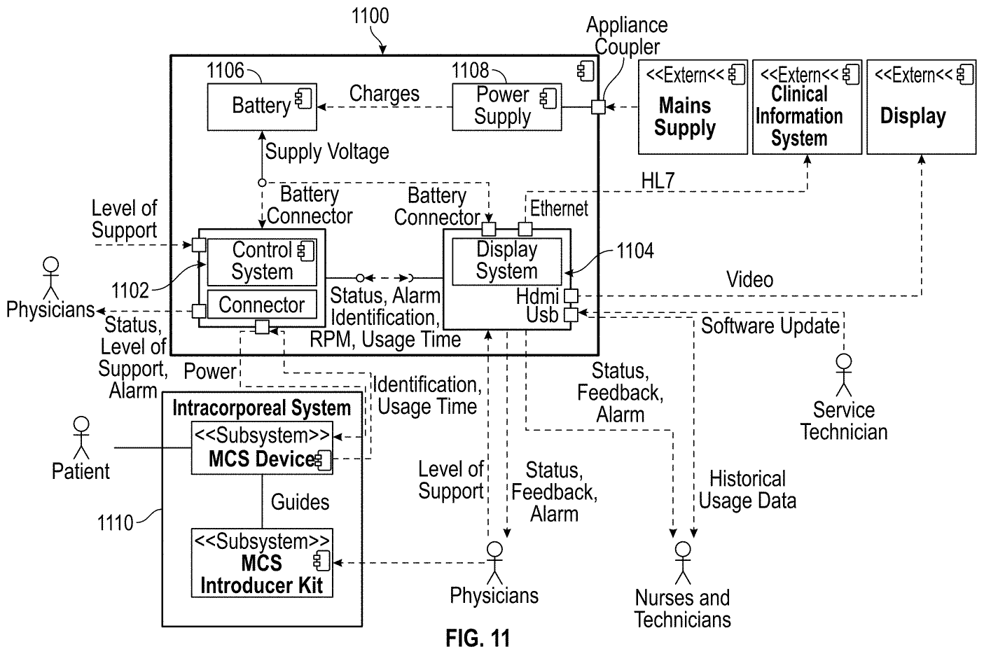

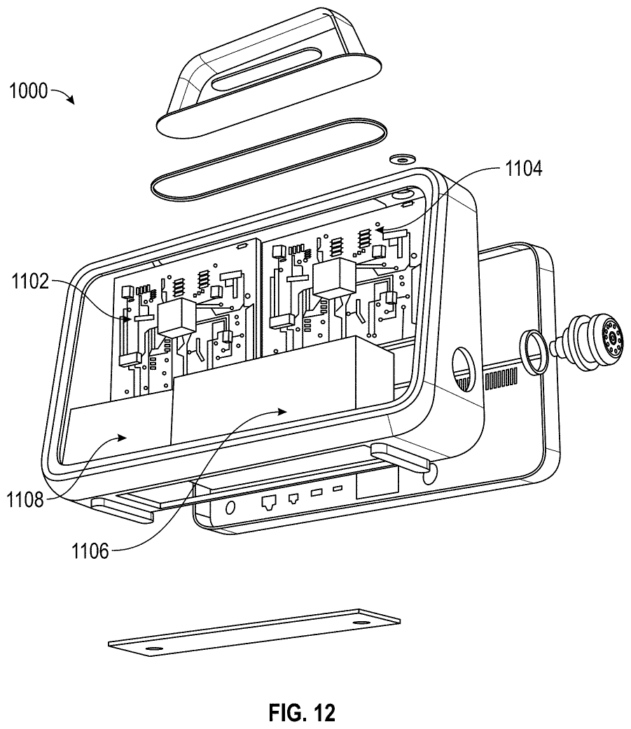

FIGS. 10A and 10B show a front and a back view of an embodiment of MCS controller or controller 1000. The controller 1000 may support operation of one or more cardiac or circulatory support systems, such as left ventricular support devices, ventricular assist devices, or MCS devices as described herein. The controller 1000 may include one more modules to provide power to the cardiac support systems. The controller 1000 may house electronic circuits to send and receive operational signals to the cardiac support system. The controller 1000 may house one or more hardware processors as described below to receive and process data, such as sensor data, from the cardiac support system. In some embodiments, the controller 1000 may have an integrated or self-contained design in which all or almost all of the components required for operation of the controller are housed within the controller. For example, any power supply components, such as transformers or AC/DC converters, may be housed within the controller 1000. As shown in FIG. 2, the controller 1000 may be wired to the pump via electronic wires extending through the catheter shaft 62 to the pump.

In some embodiments, the controller 1000 may include communications systems, or any other suitable systems, to allow the controller 1000 to be adapted to new or modified uses after construction of the controller. For example, multiple modes of wired or wireless communication can be integrated within the controller 1000 to communicate with outside technology, such as, for example, RF, wifi, and/or Bluetooth. In some embodiments, the controller 1000 may have an RFID reader. In some embodiments, the controller 1000 may have systems or components that enable syncing patient data, telemedicine, patient monitoring, real time data collection, error reporting, and/or sharing maintenance records.

The controller 1000 may include a housing for these modules that support any of the cardiac support systems described herein. The housing may further include a handle 1002 to support portability. In contrast to some of the other controllers, such as Abiomed's Impella Controller, the controller 1000 may not include components required for purging. For example, the controller 1000 does not include a cassette for purging. The cassette typically delivers rinsing fluid to the catheter. However, the cassette requires significant real estate and makes the housing bigger and heavier. Due to the design improvements described herein, such as bearing design and sealed motor discussed herein, the controller 1000 does not include a cassette. Furthermore, in some embodiments, the controller 1000 does not require a port for receiving a purging tube. Accordingly, the controller 1000 may be light and compact to support portability.

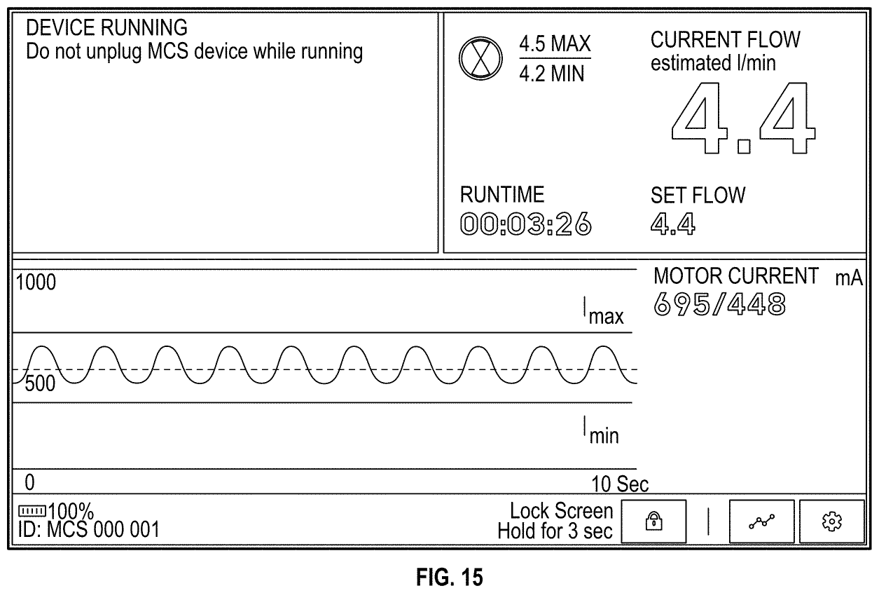

The controller may also include a cable management support 1004. In some embodiments, the cable management support 1004 is positioned on one end or side of the controller 1000. The controller 1000 may also include a mount 1006 that may support mounting the controller to a pole in a clinical environment. The mount 1006 may rotate about an axis to support horizontal or vertical clamping. The mount 1006 may be rapidly locked into the desired orientation by quick fastening with a slipping clutch. In some instances, the mount 1006 is positioned away from the cable management support 1004. Furthermore, in some embodiments, the cable management support 1004 is positioned on a left end of the controller 1000 as shown in FIG. 10A. The port 1107 (such as shown in FIG. 13) can be positioned on a side opposite from the cable management support 1004. In some instances, the control element 1008 discussed below is positioned on a side opposite from the cable management support 1004 and in close proximity to the port 1107. This may enable a user to have an improved interaction with the active components of the controller 1000. Therefore, the arrangement of all these elements in the controller 1000 as illustrated can improve operational experience and improve portability.

The controller 1000 may include a control element 1008. In some embodiment, the control element 1008 may provide a haptic feedback. The control element 1008 can include a push button rotary dial. The control element 1008 may enable a user to change parameters on the controller 1000 to control one or more processes described herein. The control element 1008 may also include status indicator 1010 as illustrated in FIG. 10A. In some embodiments, the controller 1000 may include a separate confirmation control element. Furthermore, in some embodiments, aside from the separate confirmation control element, all the parameters can be modified using a single control element 1008. The grouping of controls in a dedicated area may improve user experience.

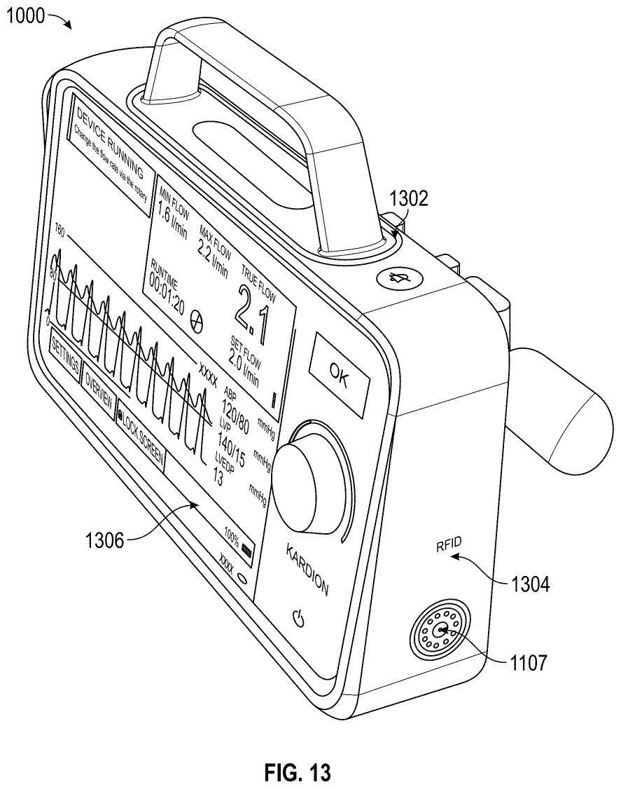

FIG. 11 illustrates a block diagram of an electronic system 1100 that can be included in the controller 1000. In some embodiments, the electronic system 1100 can include one or more circuit boards in conjunction with one or more hardware processors for controlling MCS device 1110. The electronic system 1100 can also receive signals, process signals, and transmit signals. The electronic system 1100 can further generate a display and/or alarms. The electronic system 1100 can include a control system 1102 and a display system 1104. In some embodiments, the display system 1104 can be integrated into the control system 1102 and is not separate as shown in FIG. 11. In some embodiments, it may be advantageous for the display system 1104 to be separate from the control system 1102. For example, in the event of failure of the control system 1102, the display system 1104 can serve as a backup.

The control system 1102 can include one or more hardware processors to control various aspects of the MCS device 1110. For example, the control system 1102 can control a motor of the MCS device 1110. The control system 1102 can also receive signals from the MCS device 1110 and process parameters. The parameters can include, for example, flow rate, motor current, ABP, LVP, LVEDP, etc. The control system 1102 can generate alarms and status of the electronic system 1100 and/or MCS device 1110. In some embodiments, the control system 1102 can support multiple MCS devices 1110. The control system 1102 can transmit the generated alarms or status indicators to the display system 1104. The display system 1104 can include one or more hardware processors to receive processed data from the control system 1102 and render the processed data for display on a display screen. The control system 1102 can also include a memory for storing data.

The electronic system 1100 can also include a battery 1106 that can enable its electronics systems to operate without connection to an external power supply. The power supply interface 1108 can charge the battery 1106 from the external power supply. The control system 1102 can use the battery power to supply current to the motor of the MCS device 1110.

The one or more hardware processors can include microcontrollers, digital signal processors, application specific integrated circuit (ASIC), a field programmable gate array (FPGA) or other programmable logic device, discrete gate or transistor logic, discrete hardware components, or any combination thereof designed to perform the functions described herein.

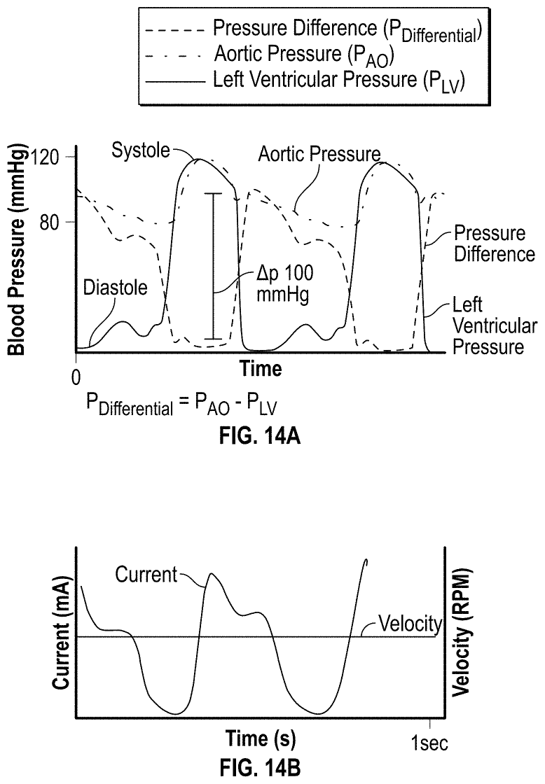

FIG. 12 is an exploded view of an embodiment of the controller 1000 having physical components corresponding to the features of the block diagram schematic of the electronics system 1100 of FIG. 11. As shown in FIG. 12, the controller 1000 may include the control system 1102 and display system 1104 including circuit boards arranged within the housing. The battery 1106 may be located within the bottom section of the housing. The power supply interface 1108 may be located within a corner of the housing.

FIG. 13 is a front perspective view of the controller 1000. In some embodiments, the controller 1000 can include an alarm feedback system, which can provide feedback to an operator regarding the operation of the MCS system. In some embodiments, the alarm feedback system can be in the form of an LED 1302 as illustrated. The LED 1302 may be positioned so that it can be seen by an operator using the controller. As illustrated, the LED 1302 is positioned around the handle 1002. Therefore, it can be seen from positions 360° around the controller. The LED 1302 may be in the form of a ring (oval, oblong, circular, or any other suitable shape) wrapping the handle 1002. Such an LED 1302 may be visualized from any direction as long as the top of the controller is viewable. The control system 1102 can generate different colors or patterns for the LED 1302 to provide various alarms or status of the controller 1000 and/or the MCS device 1110.

The controller 1000 further includes a port 1107 that may receive a cable connected to an MCS device. The port 1107 can support multiple versions of the MCS devices. The controller 1000 can also include an RFID reader 1304 on a side of the controller 1000. The RFID reader 1304 can read badges of a sales representative and operate the device according to a particular demo mode. The controller 1000 can include a glass cover 1306 that is tilted as shown in FIG. 13 to improve readability for the user.

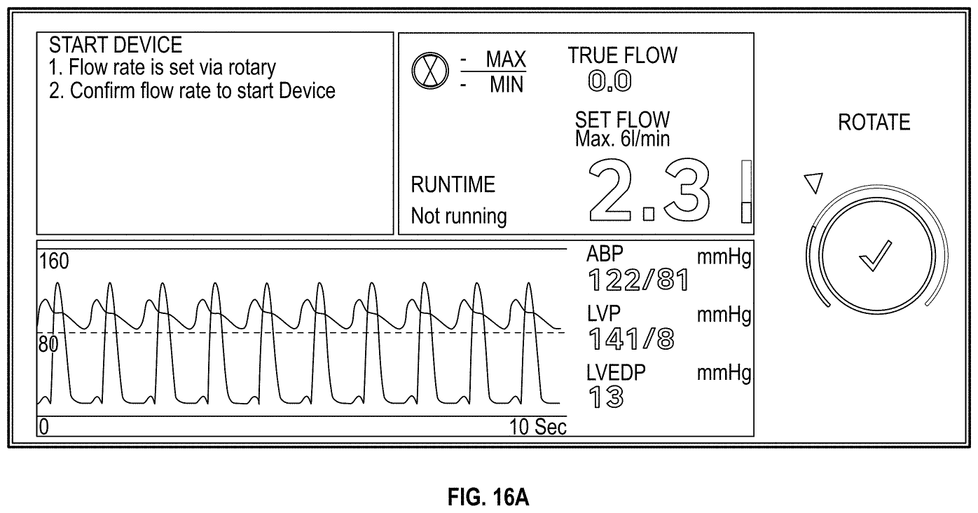

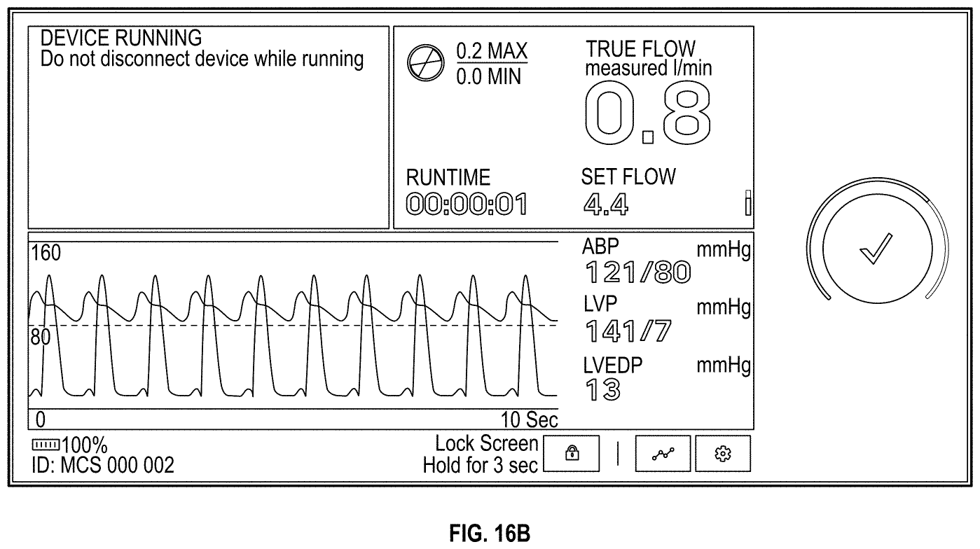

FIG. 14A illustrates a graph showing pressure differences between aortic pressure and left ventricular pressure, which may be typical pressure differences. In some instances, the MCS device 1110 can be positioned between the two different pressure levels (left ventricle and aortic arch). Therefore, the MCS device 1110 may operate against a pressure difference shown in FIG. 14A. Accordingly, the motor of the MCS device 1110 may in some instances work with the pressure and in other instances against the pressure. Therefore, it was observed that to keep the velocity of the motor, e.g. rotational speed of a motor shaft, constant or approximately stable, the current supplied to the motor would need to change based on the pressure differential.

FIG. 14B shows the applied current for a constant motor velocity. The current curve of FIG. 14B follows a similar behavior as to the pressure differential curve of FIG. 14B. In some embodiments, the control system 1102 can control a motor to run at constant velocity by varying the motor current. The variation in the motor current can be used by the control system 1102 to probe the differential pressure, and therefore physiology of the patient, operating conditions, and machine conditions.

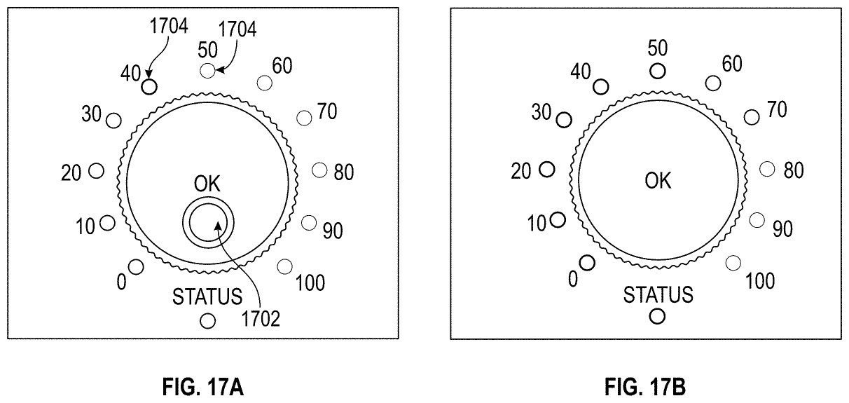

FIG. 15 illustrates an example user interface that can display flow rate parameters and motor current. The user interface can also display the parameters as a graph plotted with time. The user interface may be shown on the controller 1000, for example on the display.

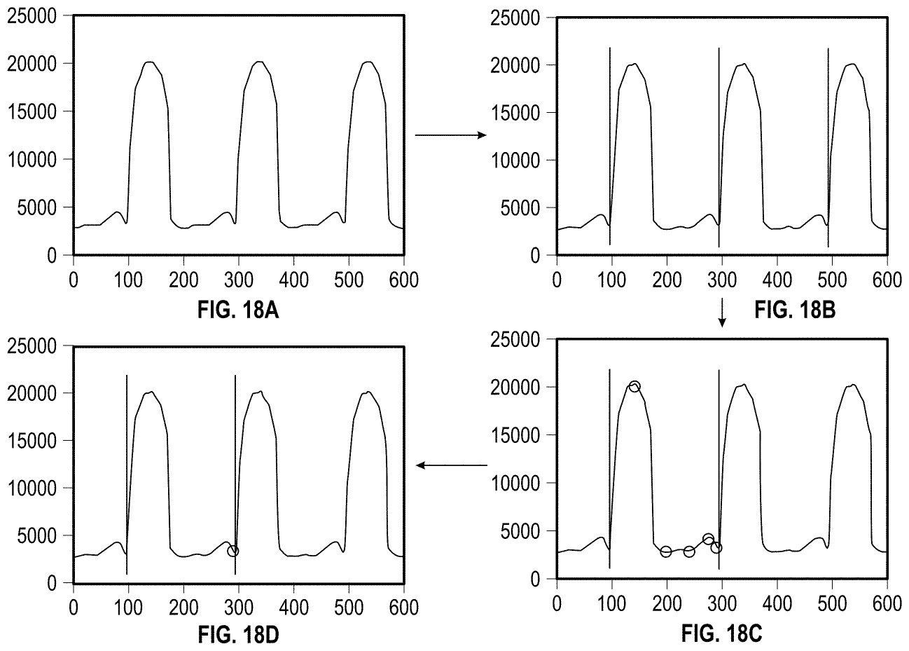

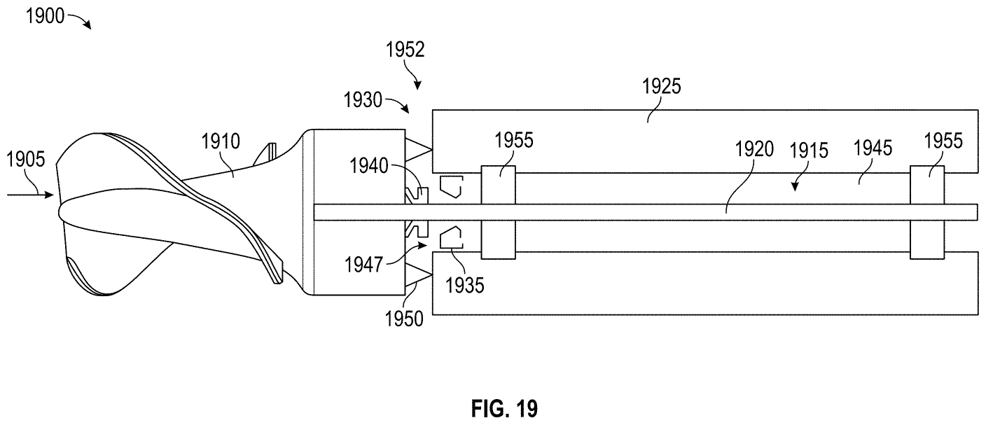

FIG. 16A illustrates an example user interface in a configuration mode where the control element 1008 can be used to modify parameters, such as setting the flow rate. The control element 1008 can include a visual feedback system directly on the knob and/or adjacent to the knob. FIG. 16B shows an example user interface during operation mode. Comparing FIGS. 16A and 16B, certain text on the user interface can be highlighted or emphasized depending on the modes. In the configuration mode, the set flow rate is enlarged. In operational mode, the flow rate is enlarged. This improves readability for the users particularly when the user interface includes several parameters.

In some embodiments, only some of the user interfaces may be available depending on the type of MCS device 1110 connected with the controller. For example, some devices discussed above may not include any sensors and may not support all the user interfaces discussed above. These sensor-less devices may be lower cost and smaller.

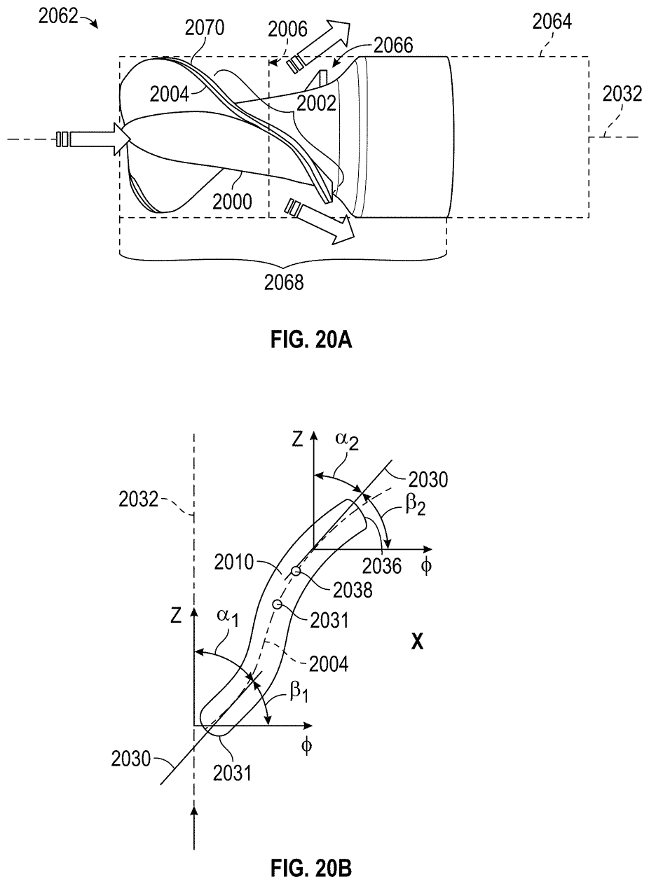

FIG. 17 illustrates an embodiment of an electronic control element 1702 and visual indicators 1704. The electronic control element 1702 can include a display on the face of the dial. Furthermore, the visual indicators 1704 can indicate status of the motor or other operating conditions as the dial is rotated.

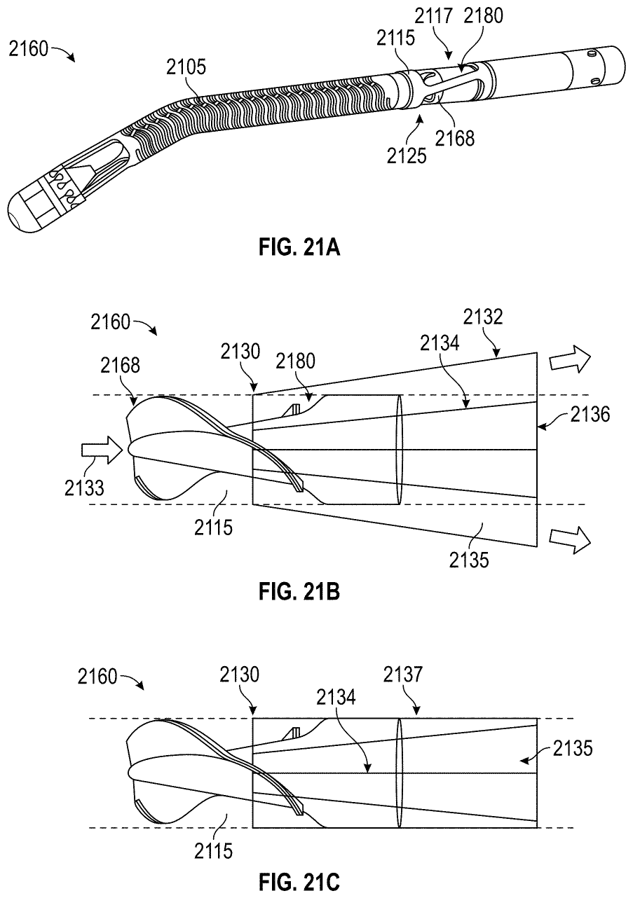

FIGS. 18A to 18D are example left ventricle (LV) pressure curves illustrating a process for determining left ventricular end-diastolic pressure (LVEDP). The control system 1102 can document the status and operational parameters, which may be transferred to an EMR system via network communications. The control system 1102 can measure left ventricular end-diastolic pressure (LVEDP). FIGS. 18A to 18D illustrate a series of steps for the determination of LVDEP from the measured LV pressure curve. FIG. 18A illustrates an example LV pressure curve measured with 100 MHz sampling rate. The control system 1102 can process the measured LV pressure curve to determine LVDEP. For example, the control system 1102 can identify a largest positive gradient in the LV curve as illustrated in FIG. 18B. This can identify the pulse value. Other techniques can be used to identify a start of a pulse. Once pulse are identified, the control system 1102 can find maxima and minima in the LV curve between 2 steep positive slopes as illustrated in FIG. 18B. This can also yield systolic and diastolic values. In some instances, the control system 1102 can identify a minimum value left of the 2^{nd} slope as illustrated in FIG. 18D. This value can represent the LVEDP determination.

As discussed above, e.g. with respect to FIG. 14B, controlling or synchronizing motor current with heart and measuring the motor current can enable the control system 1102 to probe the differential pressure through measuring current, and therefore physiological processes of the patient, operating conditions, and machine conditions. Physiological processes may include when the pump is hitting the wall of the heart. In some instances, the motor current is kept constant while measuring the change in RPM. In some instances, a separate flow or pressure sensor is not required to probe physiological processes. The motor design including a motor controller, such as the controller 1000, can enable high resolution current measurement. In some instances, a motor controller is sensorless, for example the motor controller may not include a Hall sensor. In some instances, the control system 1102 may operate the motor in a pulsatile mode to improve heart recovery.

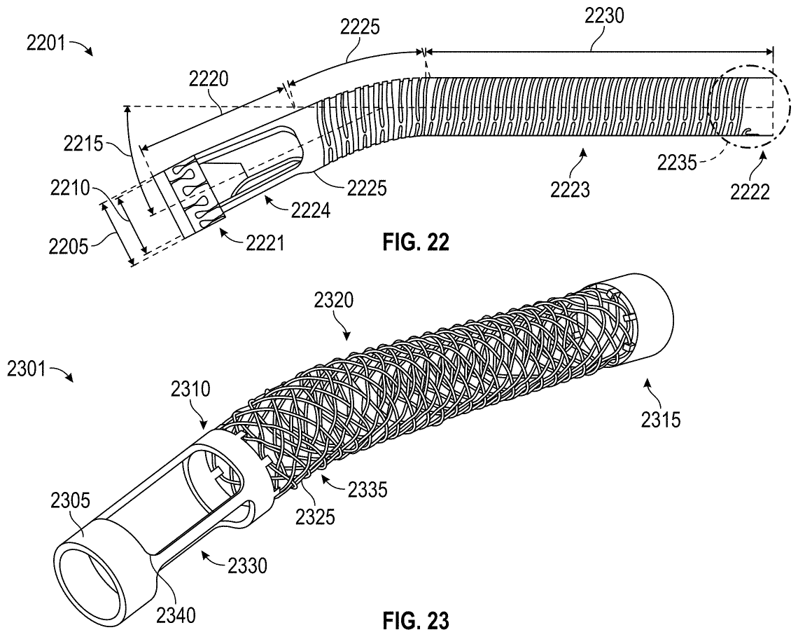

FIG. 19 shows a schematic side view of another embodiment of a pump 1900 for pumping blood 1905. The pump 1900 is designed and shaped for use in a fluid channel such as a blood vessel. The pump 1900 or features thereof may be used with any of the other pumps or features described herein, such as the pump 22, and vice versa. For example, features of the pump 1900 may be used with the pump 22 described above. In some embodiments, the pump 22 includes the motor, shaft and/or seal arrangement of the pump 1900, as further described.

The pump 1900 may have an impeller 1910, a drive device 1915 with a shaft 1920, a shaft housing 1925 and/or a sealing device 1930. The impeller 1910 may be designed to pump the fluid 1905. The drive device 1915 with shaft 1920 may be designed to drive the impeller 1910. The shaft housing 1925 may be designed to accommodate the shaft 1920 and/or the drive device 1915 and is also referred to as the “housing” in the following. The sealing device 1930 may comprise at least one casing or housing sealing element 1935 and/or one impeller sealing element 1940, which is accommodated between the drive device 1915 and the impeller 1910 and which is designed to prevent fluid 1905 from entering the drive device 1915 and/or the shaft housing 1925 during operation of the pump 1900.

According to this embodiment, the impeller 1910 may have an exemplary tapered basic body, which can be rotated around a longitudinal axis during operation of the impeller 1910. Radially around the longitudinal axis, the basic body according to this embodiment has two blades in order to generate a fluid flow or fluid suction in the fluid 1905 when the impeller 1910 rotates. For this purpose, the blades may be arranged spirally wound around an outer wall of the basic body according to this embodiment. A body of rotation of the impeller 1910 is created by the rotation of one or more so-called “B-spindles”. According to some embodiments, the impeller 1910 may have a differently shaped, for example cylindrical, basic body and/or a different number of blades or vanes. According to this embodiment, the drive device/unit 1915, which will also be referred to as “drive” in the following, has a motor 1945, for example in the form of an electric motor. According to this embodiment, the motor 1945 is coupled to the shaft 1920. The shaft 1920 is straight according to this embodiment. The shaft housing 1925 is correspondingly tubular according to this embodiment and accommodates at least the shaft 1920 or, according to this embodiment, the entire drive unit 1915 with the motor 1945 completely. According to some embodiments, the motor 1945 is arranged outside the shaft housing 1925. According to this embodiment, the housing sealing element 1935 and/or impeller sealing element 1940 is made of a strong but elastic material. In other words, the housing sealing element 1935 and/or impeller sealing element 140 has no liquid or semi-liquid material.

According to this embodiment, the housing sealing element 1935 may be attached to an inner side of the shaft housing 1925 and/or arranged around the shaft 1920. According to this embodiment, the housing sealing element 1935 may be formed as a sealing ring, according to this embodiment as a rotary shaft seal. According to this embodiment, the housing sealing element 1935 may be attached to an inlet opening 1947 of the shaft housing 1925 facing the impeller 1910. According to one embodiment, the housing sealing element 1935 may be fixed directly to the inlet opening 1947.

The additional or alternative impeller sealing element 1940 may be attached to the impeller 1910 and/or arranged around the shaft 1920 and/or the shaft housing 1925 in contact according to this embodiment. According to this embodiment, the impeller sealing element 1940 may be designed as an additional sealing ring, here an axial shaft seal. The axial shaft seal may also be described as a “V-ring”. According to this embodiment, this V-ring may have a V-shaped or plate-shaped flexible sealing lip that extends away from an annular base body of the axial shaft seal. According to this embodiment, the sealing lip is attached to the impeller 1910.

The impeller sealing element 1940 may also be preloaded towards the shaft 1920 in the mounted state according to this embodiment. A pretension may be caused by a deformation of the impeller sealing element 1940. According to some embodiments, the pump 1900 may have a spring element which causes the preload.

Furthermore, according to this embodiment, the impeller sealing element 1940 may have at least one gap sealing element 1950, which may be arranged to fluidically seal a gap 1952 between the shaft housing 1925 and the impeller 1910 in order to prevent the fluid 1905 from entering the gap 1952. According to this embodiment, the gap sealing element 1950 may be designed as an additional sealing ring. According to this embodiment, an outer diameter of the gap sealing element 1950 may be larger than an outer diameter of the impeller sealing element 1940. According to this embodiment, the impeller sealing element 1940 may be arranged coaxially with respect to the additional sealing ring in a passage opening of the additional sealing ring.

A free end of the shaft 1920 may be fixed in the impeller 1910 according to this embodiment. According to some embodiments, the free end of the shaft 1920 and the impeller 1910 may be connected without contact by means of a magnetic coupling, whereby a driving force of the motor 1945 is magnetically transferable to the impeller 1910.

The pump 1900 may also have, according to this embodiment, a bearing device 1955 for radial and/or axial bearing of the shaft 1920 in the shaft housing 1925. For this purpose, the bearing device 1955 according to this embodiment may have two bearing elements at two opposite ends of an interior of the shaft housing 1925, in which the shaft 1920 is, for example, centrally mounted. According to this embodiment, the housing sealing element 1935 may be arranged outside a space bounded by the two bearing elements.

The pump 1900 presented here may be used and shaped as a blood pump for a cardiac support system. According to one embodiment, the pump 1900 is designed as a ventricular assist device (VAD) pump for short-term implantation with a contacting radial and/or axial seal.

If the pump 1900 is used as a temporary/short-time used VAD-Pump, it is important that they can be implanted very quickly. According to this embodiment, a system as simple as possible may be used for this purpose. There may be only one or more sealing elements 1935, 1940, 1950 and liquid or partially liquid media such as flushing media or barrier media may be dispensed, or an external forced flushing for sealing or preventing blood from entering the motor.

In some embodiments, the pump 1900 may include a seal, and/or one or more features of a seal, as described herein with respect to FIGS. 30A-30C.

According to this embodiment, the pump 1900 presented here may have the electric drive in the form of the electric motor 1945, the rotating shaft 1920, the impeller 1910, the bearing device 1955, the shaft housing 1925 and/or at least one sealing element 1935, 1940, 1950, which may be firmly connected to the housing 1925 according to an embodiment in the form of the housing sealing element 1935 and has a sealing function with respect to the rotating shaft 1920 and/or the impeller 1910. Additionally or alternatively, the pump 1900 may have a sealing element in the form of the impeller sealing element 1940 and/or gap sealing element 1950, which seals the housing 1925 against the rotating impeller 1910 in the axial direction. According to this embodiment, the impeller 1910 may consist of a core with, for example, a hub and at least two or more blades. During operation of the pump 1900, the fluid 1905 may be fed axially to the impeller 1910 (suction) and discharged radially/diagonally through openings in an impeller housing of the impeller 1910 not shown here. According to this embodiment, the impeller 1910 may be firmly connected to the drive shaft 1920 of the motor 1945, which provides the required drive power. According to this embodiment, the shaft 1920 may be supported by at least one radial and/or at least one axial bearing. Optionally, the bearings may also be realized in combination with a radial-axial bearing. According to one possible embodiment, the housing 1925 may have at least one sealing element 1935 to the impeller 1910. According to another embodiment, this at least one sealing element 1940, 1950 may be attached to the impeller 1910. The seal may be of contact design, i.e. according to one embodiment, the sealing element 1925, 1940 is always in contact with the shaft 1920 and the casing 1925. Furthermore, at least one (further) sealing element 1940, which seals the shaft 1920 against the casing 1925, may be arranged optionally/alternatively. This may be designed according to an embodiment in such a way that the sealing element 1940 is preloaded towards the shaft 1920. According to one embodiment, this may be realized with a spring or according to another embodiment, it is ensured by shaping the elastic sealing element 1940. One possible design of the housing sealing element 1935 is a rotary shaft seal. The axial shaft sealing ring is a possible design of the alternative/optional sealing element 1940.

According to one embodiment, the VAD pump 1900 may have a maximum outside diameter of less than five millimeters, in another embodiment it may have an outside diameter of less than eight millimeters. According to one embodiment, the pump 1900 may be designed for a short-term use of less than 24 hours, in another embodiment for a use of less than ten days, in another embodiment for less than 28 days, and in another embodiment for less than or equal to six months.

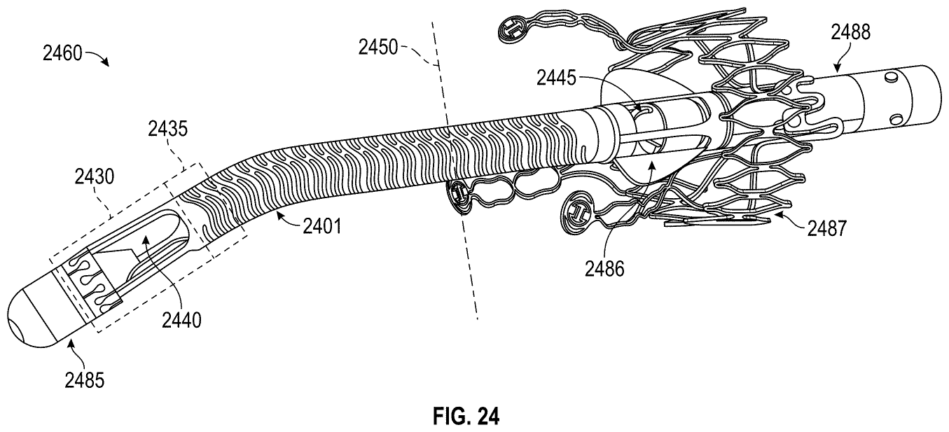

FIG. 20A shows a side view of an alternative embodiment of a pump 2062 having an embodiment of an impeller 2068. The impeller 2068 is rotatably mounted within an impeller housing, which may be a proximal end of an inlet tube or a separate housing for the impeller 2068. The impeller 2068 may face outlet openings 2066. The impeller 2068 may provide for axial suction, and radial and/or diagonal discharges, of the blood via the outlet openings 2066. The pump 2062 can include an axis of rotation 2032. The pump 2062 may rotate about an axis of rotation 2032. A motor inside the sealed motor housing 2064 may rotate the impeller 2068.

The impeller 2068 may include at least one helically wound blade 2070. The blade 2070 may ensure the efficient and gentle transport of blood. As shown in FIG. 20A, the blade 2070 may be helically wound around a hub 2000 of the pump 2062. The hub may form an inner core of the impeller 2068. A flow direction of the blood flow path is indicated by three arrows. The blood is aspirated by a pump inlet that acts as an intake opening upstream of the impeller 2068 and exits out the outlet openings 2066.

A skeleton line 2004, which may be a camber line, of the blade 2070 may have a point of inflection in a region of the upstream start of outlet openings 2066. The blade 2070 may extend from an upstream end of the pump rotor 2068 over an entire length or at least over a major part of the hub 2000. In the embodiment of FIG. 20A, the hub 2000 may have a diameter that increases in the direction of flow, so that the shape of the hub 2000 thickens along the direction of flow. This shape of the hub may facilitate a radial and/or diagonal discharge of the blood.

The blade 2070 may include a vane section 2002 having a wave-shaped vane curvature (e.g., a wavy blade curvature), which is defined by a multiple curved portions of a skeleton line 2004 of the blade 2070. As discussed herein, a wave-shaped curvature of the blade 2070 may refer to a change in curvature of the vane section 2002 associated with at least one sign change, for example positive or negative concavity/convexity. At least one section of the blade 2070 and/or the entire vane section 2002, or one portion of the vane section 2002, may be located radially inwardly of the outlet openings 2066. The vane section 2002 may be at least partially in the region of a flow-facing edge 2006 of the outlet opening 166. The vane section 2002 may represent one or more transitions between a convex and a concave curvature. The outlet opening(s) 2066 of the tubular housing of the circulatory support device may at least partially overlap the blade section 2002 of the impeller 2068 having the wavy blade curvature.

In certain embodiments, the impeller 2068 may comprise two blades 2070, which are wound in the same direction around the hub 2000. Each blade 2070 may have the vane section 2002. In some embodiments, the impeller 2068 may include more than two blade elements 2070, such as three, four, five, six or more. The pump 2062, or other pumps described herein, may have additional features or modifications, such as those described in PCT Publication No. WO 2019/229223, filed May 30, 2019, titled AXIAL-FLOW PUMP FOR A VENTRICULAR ASSIST DEVICE AND METHOD FOR PRODUCING AN AXIAL-FLOW PUMP FOR A VENTRICULAR ASSIST DEVICE, and/or described in U.S. patent application Ser. No. 17/057,252, filed Jun. 18, 2021, titled AXIAL-FLOW PUMP FOR A VENTRICULAR ASSIST DEVICE AND METHOD FOR PRODUCING AN AXIAL-FLOW PUMP FOR A VENTRICULAR ASSIST DEVICE, the disclosures of each of which is hereby incorporated by reference herein in its entirety for all purposes and forms a part of this specification.

FIG. 20B shows a schematic illustration of an example unwinding of the skeleton line 2004 of the blade element 2010 of FIG. 20A. Two pairs of blade angles α₁, β₁ and α₂, β₂ are drawn in as an example, each of which represents a tangent slope of a tangent 2030 representing the curvature of the skeleton line 2004. Each tangent 2030 is drawn into a cylindrical coordinate system with a z-axis parallel to an axis of rotation 2032 of the pump rotor and a Φ-axis perpendicular to the z-axis. The Φ-axis represents a circumferential direction of the pump rotor.

The tangent slope initially increases in the flow direction, indicated by a vertical arrow, and then decreases again. According to this design example, the tangent slope initially increases continuously from a blade leading edge 2034 to a blade trailing edge 2036 of the blade element 2010 and, upon reaching the inflection point 2031 of the skeleton line 2004, decreases again. A point 2038 marks a position of a flow discharge via the outlet openings of the pump housing, more precisely a start of the flow discharge in axial direction. The objective here is to ensure that the inflection point 2031 and the point 2038 of the start of the flow discharge are in close proximity.

As already described, according to a design example, the pump rotor may be realized with at least two blade elements 2010. The conveying medium is delivered axially to or is drawn in by the pump rotor and expelled radially and/or diagonally through one or more outlet openings 2066 in the pump housing. The blade elements 2010 are configured such that the angle α between the tangent 2030 formed with a blade surface or the skeleton line 2004 and the axis of rotation 2032 or the z-axis changes in axial direction. The angle β between the circumferential direction or the Φ-axis and the blade surface or the skeleton line 2004 changes to the opposite extent. The angle β changes such that, at least in the region of the largest diameter of the pump rotor, i.e. in a section in the region of the blade tips of the blade elements 2010, from the start of the pump rotor, i.e. from the blade leading edge 2034, it increases in flow direction. The angle β in particular assumes its greatest value in the region of the start of the flow discharge 2038 or in close proximity thereof, at least in the region of the largest diameter of the pump rotor, i.e. in a section in the region of the blade tips of the blade elements 2010.

In some embodiments, the impeller 2068 comprises a blade element 2010 having a profile with skeleton lines 2004, and a curvature of each of the skeleton lines 2004 when unwound into a plane increases along the axis of rotation 2032 in a direction starting from the pump intake section towards the outlet opening 2066 to an inflection point 2031 at which a blade angle β of the blade element 2010 is at a maximum, and the curvature of each of the skeleton lines 2004 decreases after the inflection point 2031. Further, in some cases a region of the impeller 2068 located radially relative to the axis of rotation 2032 of the impeller 2068 has a blade height SH of the blade element 2010 defined relative to a maximum blade height SHMAX such that 25%≤SH/SHMAX≤100%, with the inflection point 2031 of each of the skeleton lines 2004 located in a region of an upstream edge of an outlet opening 2066 of an inlet tube of the tubular housing. The blade element 2010 may have other features or modifications, for example those described in PCT Publication No. WO 2019/229223, filed May 30, 2019, titled AXIAL-FLOW PUMP FOR A VENTRICULAR ASSIST DEVICE AND METHOD FOR PRODUCING AN AXIAL-FLOW PUMP FOR A VENTRICULAR ASSIST DEVICE, and/or described in U.S. patent application Ser. No. 17/057,252, filed Jun. 18, 2021, titled AXIAL-FLOW PUMP FOR A VENTRICULAR ASSIST DEVICE AND METHOD FOR PRODUCING AN AXIAL-FLOW PUMP FOR A VENTRICULAR ASSIST DEVICE, the disclosures of each of which is hereby incorporated by reference herein in its entirety for all purposes and forms a part of this specification.

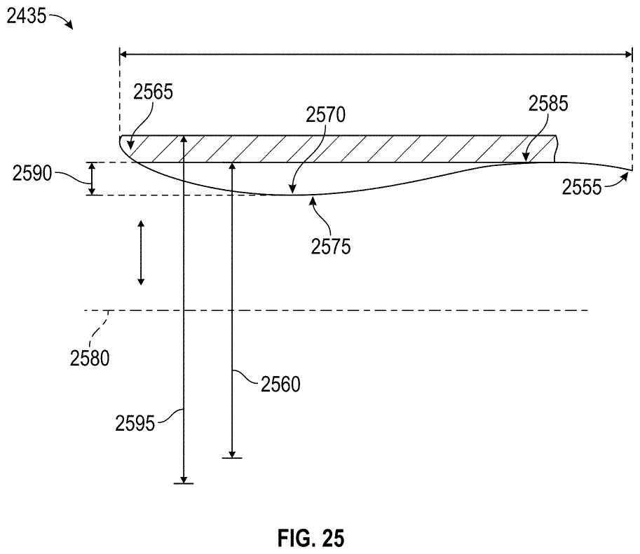

FIGS. 21A-C show an embodiment of a pump region 2160 having a tubular housing that includes an impeller housing 2115. The pump region 2160 may include a pump 2117 having an alternative embodiment of an impeller housing 2115. In some embodiments, the impeller housing 2115 may include a diffuser, as further described. The pump region 2160 or features thereof may be used with any of the MCS systems or pumps described herein. The pump region 2160 may be arranged in a minimally invasive manner through a transfemoral or transaortic catheter in an aorta and/or at least partially in a ventricle. As described herein, the pump region 2160 can include a blood pump 2117 for a heart support system. A maximum external diameter of the pump region 2160 shown in FIG. 21 may be less than ten millimeters (e.g., less than or equal to 7 mm, less than or equal to 5 mm). The pump 2117 may have an axial design including an impeller 2168 against which axial flow occurs. The axial design of the pump 2117 may facilitate a pump region 2160 having a maximum external diameter of less than 10 mm.

Blood flows during the operation of the pump device 2160 through an inlet tube 2105 and is expelled through outlet openings 2180 within a circumference of an impeller housing 2115 of the pump 2117 in order to be fed to the aorta (e.g., from the left ventricle, across the aortic valve, and to the aorta). This is made possible by and implemented in the embodiment of FIG. 21A, in which the impeller 2168 is completely enclosed in a first section by the impeller housing 2115, which is in the form of a cylindrical/tubular impeller housing, and is interrupted in a second section by the outlet openings 2180 in the impeller housing 2115. A transition between these two sections is characterized by a beginning 2125 of the outlet openings 2180.

As shown in FIGS. 21B-C, some embodiments of the MCS system may further comprise a diffuser 2130 configured to couple with the tubular housing. The outlet openings 2180 may be configured to facilitate outflow of blood from the tubular housing (e.g., from the inlet tube 2105 and/or from the impeller housing 2115) of the pump region 2160. The diffuser 2130 may be configured to guide the blood transversely to the outlet opening 2180 after the blood has passed through the outlet opening 2180.

As shown in FIG. 21B and according to some embodiments, the diffuser 2130 may be arranged circumferentially around the impeller housing 2115. In the operating position 2132, a lateral surface of the diffuser 2130 may have a cross-sectional area that increases in the flow direction 2133 (see arrows) of the blood. In some embodiments, the diffuser 2130 itself can also have a cross-sectional area that increases in the flow direction 2133 of the blood. In this case, the diffuser 2130 may have the shape of a truncated cone in the operating position 2132. The diffuser 2130 may have a support structure with at least one strut 2134 and/or a flexible jacket 2135. As shown in the embodiment of FIG. 21B, the diffuser 2130 has a plurality of struts 2134.

The diffuser 2130 may be formed to be transferable from a rest position 2137 (shown in FIG. 21C) to the operating position 2132 (shown in FIG. 21B) and/or from the operating position 2132 to the rest position 2137, wherein the diffuser 2130 is formed so that it can be folded out from the rest position 2137 to the operating position 2132. The diffuser 2130 may produce an improved flow routing and lower pressure losses as well as an increase in pump efficiency.

The diffuser 2130 may be permanently or detachably connected to the impeller housing 2115. In some cases, the diffuser 2130 is configured to be flexible, crimpable, foldable, and/or unfoldable. This configuration may offer the advantage that in the folded or crimped state, it can nestle closely to the impeller housing 2115 and thus allows minimally invasive implantation. The diffuser 2130 may be configured with a support structure with several struts 2134 made of a shape memory material (e.g., Nitinol) as well as a flexible jacket 2135. The flexible jacket 2135 may be completely or at least partially closed in the circumferential direction and may be made of silicone and/or PU and/or may be permanently or detachably connected to the support structure. Together with the support structure in the unfolded state shown in FIG. 21B, the lateral surface can serve for flow routing of the blood in order to reduce losses when the blood flows out of the outlet opening(s) 2180. The diffuser 2130 may have a lateral surface that, in the unfolded state, encloses a cross-sectional area increasing, i.e., divergent, in the main flow direction 2133, i.e., in the direction of the axis of the axis of rotation of the impeller 2168. A downstream discharge surface 2136 of the diffuser 2130 may therefore be larger than a connection surface, arranged opposite the discharge surface 2136, of the diffuser 2130 with the impeller housing 2115. In this case, the diffuser 2130 or at least its lateral surface may be configured in the form of a truncated cone. The diffuser 2130 may comprise other shapes and/or configurations in the operating position 2132, such as a funnel shape, a dome shape, an umbrella shape, an inverted bell shape, a bell shape, a bowl shape, and/or it may have a convex, concave, stepped, or angular discharge surface.

FIG. 21C shows the diffuser 2130 in a rest position 2137. In the rest position 2137, the diffuser 2130 may be configured to nestle closely to the impeller housing 2115 and can thus be minimally invasively implanted.

The pump 2117, diffusers 2130, other pumps or diffusers described herein, or features thereof, may have additional features or modifications, such as those described in PCT Publication No. WO 2019/229214, filed May 30, 2019, titled PUMP HOUSING DEVICE, METHOD FOR PRODUCING A PUMP HOUSING DEVICE, AND PUMP HAVING A PUMP HOUSING DEVICE, and/or described in U.S. patent application Ser. No. 17/057,548, filed May 19, 2021, titled PUMP HOUSING DEVICE, METHOD FOR PRODUCING A PUMP HOUSING DEVICE, AND PUMP HAVING A PUMP HOUSING DEVICE, the disclosures of each of which is hereby incorporated by reference herein in its entirety for all purposes and forms a part of this specification.

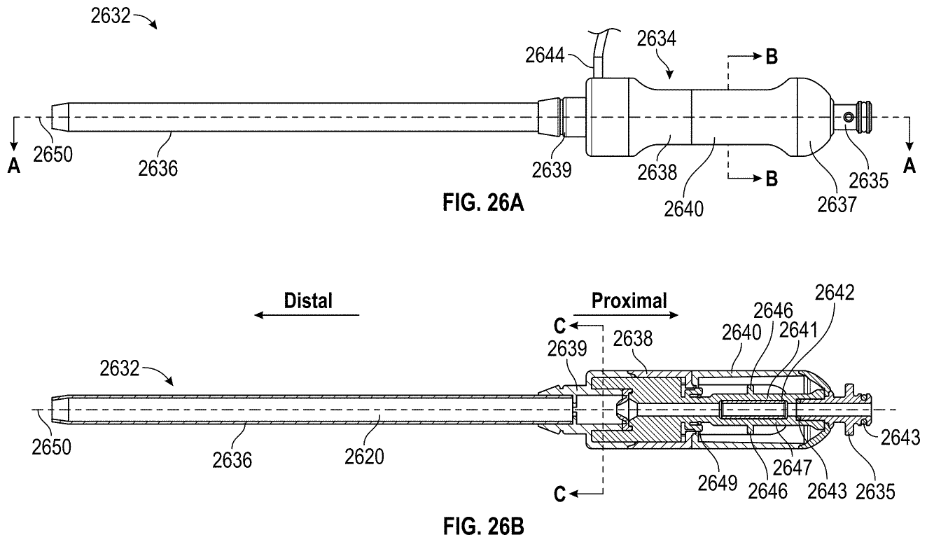

FIG. 22 is a side view of an alternative embodiment of an inlet tube 2201 of an MCS system. The inlet tube 2201 may have a main body 2225. The inlet tube 2201 can include a first connection section 2221 (which can also be referred to as a first attachment section herein) at a first end (e.g., distal end) of the inlet tube main body that may connect/attach the inlet tube 2201 to a distal tip and/or a head unit of the circulatory support device. In some embodiments, the first connection section 2221 may be configured to connect to a distal tip and/or a head unit in a form-locking and/or force-locking manner. The inlet tube 2201 may also include a second connection section 2222 (which can also be referred to as a second attachment section herein) at a second end (e.g., proximal end) of the inlet tube main body. The second connection section 2222 may connect the inlet tube 2201 to a pump outlet. In some cases, the second connection section 2222 may connect the inlet tube 2201 to an impeller housing. In some embodiments, the second connection section 2222 may connect the inlet tube 2201 to a motor housing. The main body 2225 of the inlet tube 2201 may also include a structural section 2223 extending between the second connection section 2222 and the first connection section 2221. In some embodiments, the structural section 2223 may extend between a pump inlet 2224 and the second connection section 2222.

In some embodiments, the structural section 2223 can include one or more stiffening recesses that can change the rigidity of the inlet tube 2201. The stiffening recesses may extend over part of the structural section 2223 or over the entire structural section 2223. The stiffening recesses may be arranged in a helical circumferential manner. The stiffening recesses may be in the form of slots.

FIG. 22 further includes geometric reference markings for illustrating exemplary dimensions of the inlet tube 2201. At the first connection section 2221, the inlet tube 2201 may have an inner diameter of 6.5 millimeters (or between 4.5 to 8.5 millimeters) shown by the mark 2205. The outer diameter shown in this area by the mark 2210 may be 7 millimeters (or between 5 mm to 9 mm). The angle of the bend indicated by the mark 2215 may be 26 degrees (or between 16 degrees to 36 degrees). The marking 2220 may be a length of 15 millimeters (or between 10 millimeters and 20 millimeters) of a region of the inlet tube 2201 that includes the first connection section 2221 and the pump inlet 2224, as well as a region of the structural section 2223 with the recess closest to the pump inlet 2224. In some embodiments, the first connection section 2221 is part of the pump inlet 2224. An adjacent bent portion of the structural section 2223, which may be inclined with respect to the longitudinal axis of the inlet tube 2201, may have a length of 14 millimeters, as shown by the mark 2225. The adjacent portion of the inlet tube 2201 shown by the mark 2230 includes a remainder of the structural section 2223 and the second connection section 2222. The inlet tube 2201, or any other inlet tube described herein, may have additional features or modifications, such as those described in PCT Publication No. WO 2019/229210, filed May 30, 2019, titled LINE DEVICE FOR CONDUCTING A BLOOD FLOW FOR A HEART SUPPORT SYSTEM, AND PRODUCTION AND ASSEMBLY METHOD, and/or described in U.S. patent application Ser. No. 17/057,355, filed May 18, 2021, titled LINE DEVICE FOR CONDUCTING A BLOOD FLOW FOR A HEART SUPPORT SYSTEM, AND PRODUCTION AND ASSEMBLY METHOD, the disclosures of each of which is hereby incorporated by reference herein in its entirety for all purposes and forms a part of this specification.

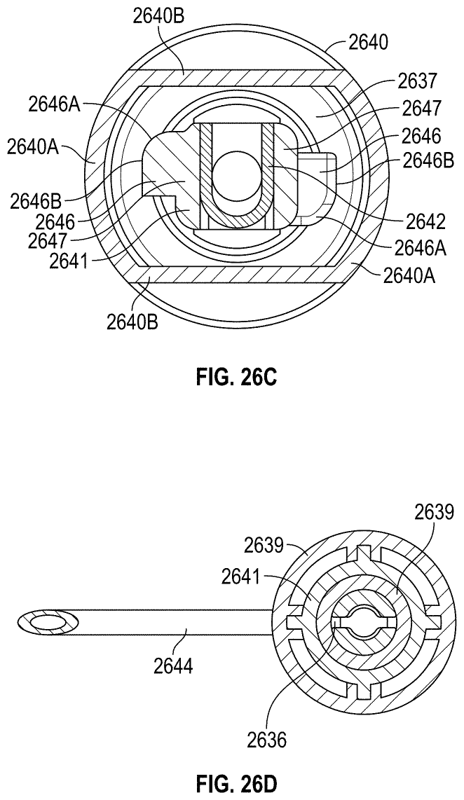

FIG. 23 is a perspective view of an alternative embodiment of an inlet 2301 of an MCS system. The inlet tube 2301 may be used with any of the pumps or MCS systems described herein. The inlet tube 2301 may be in the form of a mesh or braid suction hose. The inlet tube 2301 has a main body 2305. The main body 2305 may have at a first end, a first connection section 2310 for connecting the inlet tube 2301 to a distal tip, and at a second end a second connection section 2315 for connecting the inlet tube 2301 to a pump outlet. The pump inlet 2330 may have at least one inlet opening 2340 cut out or formed in the first connection section 2310. The inlet opening 2340 may be implemented as a multi-part window. The pump inlet 2330 may comprise three rectangular-shaped inlet openings 2340, which are rounded off in the direction of the braid section 2320 in the form of an arc of a circle.

The main body 2305 may have a braid section (which can also be referred to as a mesh section) 2320 between the connection sections 2310 and 2315. The braid section 2320 has a braid structure (which can also be referred to as a mesh structure) 2335 formed from at least one braided wire (which can also be referred to as a mesh wire) 2325. The main body 2305 has a pump inlet 2330 arranged in the first connection section 2310 for introducing the blood flow into the base/main body 2305. The inlet tube 2301 is shaped/configured to be connectable to adjacent components of the circulatory support system. The braid structure 2335 may be shaped as a diamond lattice. For this purpose, the at least one braided wire 2325 may be braided as a lattice and has a plurality of diamond meshes which form the braid structure 2335. The braided flow channel may be a braid section 2320. The braid section 2320 may be formed from a shape memory material. The inlet tube 2301 may be completely formed from nitinol. By using nitinol, the inlet tube 2301 may be not only suitable for short-term use, but also for a service life of over 10 years. Nitinol may combine the advantages of biocompatibility and the shape memory property, which makes it possible to implement complex structures in a small installation space, as in the braid section 2320 shown in FIG. 23.

The braid section 2320 may be perforated at the connection sections 2310 and 2315. For this purpose, the connection sections 2310 and 2315 may have a fastening element for threading in a section of the braided wire 2325. Additionally or alternatively, the braid section 2320 may be glued or soldered to the connection sections 2310 and 2315.

The braid section 2320 may extend over at least half of the inlet tube 2301 in order to adjust the rigidity of the inlet tube 2301. The inlet tube 2301 may be shaped to enable transfemoral surgery (access via the groin). The inlet tube 2301 may thus be flexible enough to be able to be pushed through the aortic arch, and also have a rigidity so that it can be pushed through the blood vessels in the axial direction without kinking. The relevant requirements for flexibility and rigidity of the inlet tube 2301 may be set by means of the shaping of the braid section 2320. The design of the braid structure may determine the ratio of flexibility and rigidity. Variables affecting the ratio of flexibility and rigidity include the number of wire tracks of the at least one braided wire 2325, a stiffness and a material thickness of the at least one braided wire 2325, and the braid pattern of the braid structure 2335.

The higher the number of wire tracks of the at least one braided wire 2325, the more rigid the braid structure 2335 may be. The braided wire 2325 may comprise, for example, 12 to 24 wire tracks. The larger the wire diameter of the braided wire 2325, the stiffer the braid structure 2335 may be. The wire diameter may be between 0.1 millimeter and 0.3 millimeter, for example. In addition, the material properties of the braided wire 2325 are important: the higher the modulus of elasticity of the braided wire 2325, the more rigid the braid structure 2335 may be. The braided wire 2325 may have an elasticity between 74 GPa and 83 GPa, for example. The type of braid of the braid structure 2335 is also significant: the closer-meshed the braid, the stiffer it may be.

In the embodiment shown in FIG. 23, the inlet tube 2301 may be bent in the direction of the first connection section 2310, the bend being shaped, for example, as an obtuse angle with respect to a longitudinal axis of the inlet tube 2301. The braid section 2320 may be bent at an obtuse angle at a bending point. The bend may be realized by heat treatment of the nitinol braid section 2320. Due to the shape-memory properties of the nitinol, the inlet tube 2301 can be formed with a curve shape of the braid section 2320 corresponding to the human anatomy in order to enable the inlet opening of the pump inlet 2330 of the first connection section 2310 to be positioned in the center of the heart chamber. The inlet tube 2301, or any other inlet tube described herein, may have additional features or modifications, such as those described in PCT Publication No. WO 2019/229211, filed May 30, 2019, titled LINE DEVICE FOR CONDUCTING A BLOOD FLOW FOR A HEART SUPPORT SYSTEM, HEART SUPPORT SYSTEM, AND METHOD FOR PRODUCING A LINE DEVICE, and/or described in U.S. patent application Ser. No. 17/057,411, filed Jun. 1, 2021, titled LINE DEVICE FOR CONDUCTING A BLOOD FLOW FOR A HEART SUPPORT SYSTEM, HEART SUPPORT SYSTEM, AND METHOD FOR PRODUCING A LINE DEVICE, the entire contents of each of which is hereby incorporated by reference herein in its entirety for all purposes and forms a part of this specification

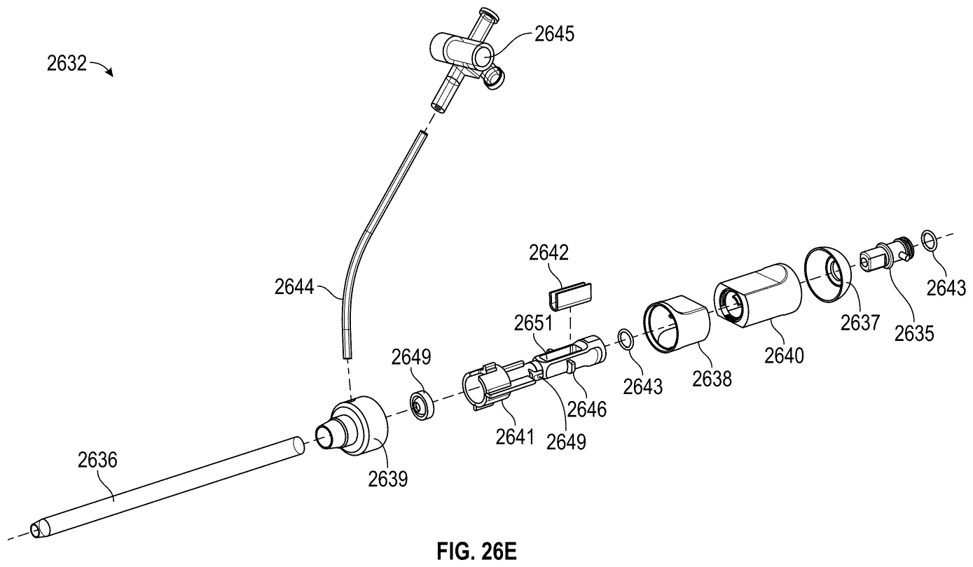

FIG. 24 is a perspective view of an alternative embodiment of a pump region 2460 of an MCS system. The pump region 2460 or features thereof may be used with any pump region or MCS system described herein. The pump region 2460 has an inlet tube 2401. The elongated, axial design of the pump region 2460, shown in FIG. 24 with an essentially constant outer diameter, enables transfemoral or transaortic implantation of the pump region 2460 for placement by means of a catheter in a blood vessel, for example the aorta.

According to the shape for the aortic valve position, the inlet tube 2401 has, for example, an incline or curvature of the longitudinal axis and thus a slightly curved shape. In addition to the inlet tube 2401, the pump region 2460 includes a pump unit 2486. The pump region 2460 may also include a distal tip 2485, a housing section 2488, and/or an anchoring frame 2487. The inlet tube 2401 maybe arranged between the distal tip 2485 and the pump unit 2486. The pump unit 2486 is connected at an end remote from the inlet tube 2401 to the housing section 2488 to which the anchoring frame 2487 is attached.

The inlet tube 2401 may be designed to guide fluid flow to the pump unit 2486 of the pump region 2460. The inlet tube 2401 may comprise a pump inlet 2430 and a contour section 2435. The pump inlet 2430 may have at least one inlet opening 2440 for introducing the fluid flow into the inlet tube 2401. At least one inlet edge of the inlet opening 2440 of the pump inlet 2430 may be rounded. The inlet opening 2440 may be designed, for example, as a window-shaped inlet opening cut into or formed within the pump inlet 2430. The contour section 2435 may have an inner surface contour. The contour section 2435 is arranged adjacent to the pump inlet 2430. In the flow direction, the inside diameter of the contour section 2435 at a first position is greater than the inside diameter at a second position. Thus, in some embodiments, the inlet tube 2401 may have a reduced diameter section at the distal end of the inlet tube 2401. The inner surface contour has a rounding to reduce the inner diameter at the second position. A length of the contour section 2435 may correspond to a radius of the inlet tube 2401 within a tolerance range. The tolerance range may be a deviation of a maximum of twenty percent from the radius of the inlet tube.

In FIG. 24, the pump inlet 2430 and the contour section 2435 are shown marked by way of example. In particular, the contour section 2435 may be a smaller or larger portion of the inlet tube 2401 than shown in FIG. 24. When implanted, the pump inlet 2430 and the contour section 2435 are arranged in the left ventricle. Another section of the inlet tube 2401 is led through the aortic valve, and a section of pump region 2460 with the pump unit 2486 is arranged in a section of the aorta when implanted. A pump outlet 2445 in the area of the pump unit 2486 guides the fluid flow conveyed through the inlet tube 2401 into the aorta. The marking 2450 shows, by way of example, a position of a heart valve, for example the aortic valve, through which the inlet tube 2401 is passed in order to position the pump region 2460.

A circulatory support system that is limited in terms of installation space, such as a circulatory support system having the pump region 2460 shown here by way of example, which can be implanted in a minimally invasive manner, has a comparatively low power consumption at a certain pump efficiency. The efficiency is limited by the friction in the pump of the pump unit 2486. The pressure loss or the friction in the inlet tube 2401 when the fluid flow is directed from the inlet opening 2440 of the pump inlet 2430 in the heart chamber to the pump unit 2486 can be affected by the shape of the inlet tube 2401. For this purpose, the inlet edges of the inlet opening 2440 may be rounded in order to reduce the pressure loss. This alone may not prevent the flow separation. The flow separation may be suppressed and thus the pressure loss can be reduced by an inlet inner surface contour formed according to the approach presented here in the form of the contour section 2435.

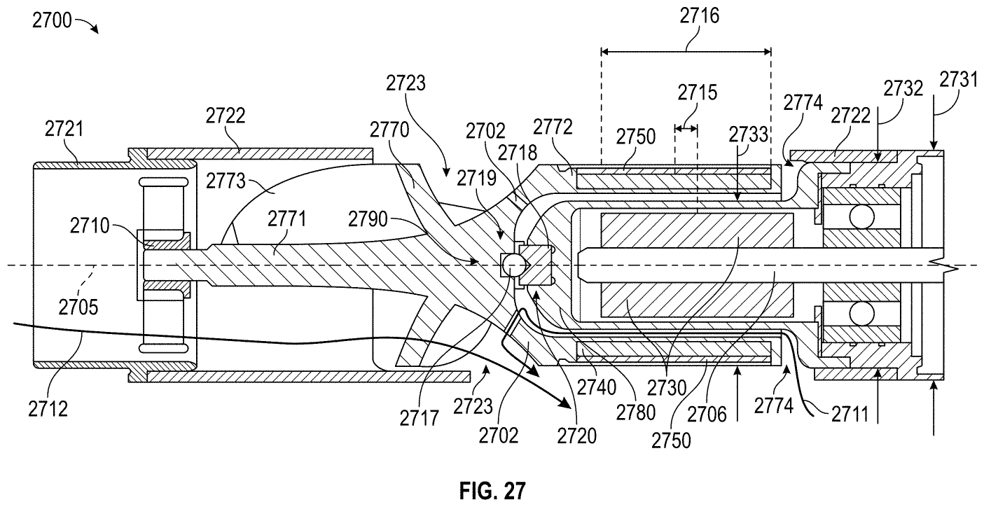

FIG. 25 is a partial cross sectional view of a contour section 2435 of the inlet tube 2401. Exemplary dimensional relationships of the contour section 2435 and the inner surface contour 2555 are shown. An axial section of one half of the contour section 2435 is shown. The inner diameter 2560 of the contour section 2435 may be larger at a first position 2565 than the inner diameter 2560 at a second position 2570. The inner surface contour 2555 may have a rounding 2575 in the form of an axially arcuate inner wall profile in order to reduce the inner diameter 2560 at the second position 2570. The first position 2565 may mark a point of the contour section 2435 along a longitudinal axis of the contour section 2435, and the second position 2570 may mark a further point of the contour section 2435 along the longitudinal axis. The second position 2570 may be downstream of the first position 2565. In the exemplary embodiment shown here, the longitudinal axis corresponds to an axis of rotation 2580 of the contour section 2435.

The first position 2565 may be arranged in the contour section 2435 between the pump inlet and the second position 2570. With regard to a flow direction of the fluid flow introduced through the pump inlet, which is directed in the direction of the pump unit through the inlet tube and thus through the contour section 2435, the first position 2565 is arranged upstream of the second position 2570. In addition, in the embodiment of FIG. 25, the inner diameter of the contour section 2435 at a third position 2585 is greater than the inner diameter at the second position 2570. The third position 2585 is downstream of the first and second positions 2565, 2570.

An inner radius of the contour section 2435 at the second position 2570 may be at most one fifth smaller than the inner radius at the first position 2565. In FIG. 25, this is shown by the marking 2590, which marks a fifth of the inner radius. Correspondingly, the rounding 2575 of the inner surface contour 2555 is designed at most as a convex bulge in the region of one fifth of the inner radius, which the marking 2590 additionally illustrates.

In some embodiments, the inner surface contour 2555 may be designed to be rotationally symmetrical. A part of the contour section 2435, which is opposite the part of the inner surface contour 2555 shown in FIG. 25 in relation to the axis of rotation 2580, accordingly has a rotation of the inside surface contour 2555 which is symmetrical. By means of the formation of the contour section 2435 and the inner surface contour 2555 shown in FIG. 25, it is possible to reduce or suppress flow detachments of the fluid flow in the inlet tube, which would otherwise form downstream of the inlet edges. In this case, an outer diameter 2595 of the contour section 2435 remains constant, and there is advantageously no increase in the installation space of the inlet line. The pressure loss of fluid flow can be reduced by means of the embodiment of the contour section 2435 shown in FIG. 25 with the inner surface contour 2555. The inlet flow and thus the flow behavior of the fluid flow are only directed locally through the contour section 2435.

The contour section 2435 may have a length which in some embodiments corresponds to a maximum of twice the inner diameter of the inlet tube. Due to the shape of the contour section 2435, the pressure loss of the fluid flow is lower further downstream than in an inlet tube with a constant inner diameter without an inner surface contour, since a suppression or reduction of the separation results in less turbulence downstream. The inner surface contour 2555 is shaped in such a way that the flow separation is largely suppressed over a length of up to four times the radius of the inlet tube. The local outer diameter 2595 of the inlet tube is limited by a prescribed wall thickness. Adjacent to the inlet opening of the pump inlet, the inlet edge is rounded convexly in order to reduce the flow separation. An optimization of the shape of the inner surface contour 2555, such as the shape shown in FIG. 25, is optionally rotationally symmetrical or, alternatively, independent of the angle of rotation.

In the embodiment of FIG. 25, an optimization of the contour profile of the inner surface contour 2555 may form two concave and one convex section, regardless of the described inlet edge rounding, with a constant wall thickness, as shown in FIG. 25 with reference to the first position 2565, the second position 2570, the third position 2585, and rounding 2575. To this end, the inner wall contour is optionally shaped in such a way that locally an inner wall radius of up to four fifths based on the inner wall radius is achieved with a constant wall thickness of the contour section 2435.

In some embodiments, the pump region 2460 comprises a tubular housing with a feed head portion (e.g., at the pump inlet 2430) with at least one introduction opening (e.g., inlet opening 2440) for receiving the fluid flow into the feed line (e.g., inlet tube 2401). The tubular housing may also include a contoured portion (e.g., contour section 2435) disposed adjacent to the feed head portion (e.g., pump inlet 2430) with an inner surface contour (e.g., surface contour 2555). The inner surface contour can include a first inner diameter at a first position 2565, a second inner diameter at a second position 2570, and a third inner diameter at a third position 2585. The first inner diameter can be greater than the second inner diameter, and the third inner diameter can be greater than the second inner diameter. The first inner diameter may comprise a maximum inner diameter of the contoured portion (e.g., contour section 2435) and the second inner diameter may comprise a minimum inner diameter of the contoured portion (e.g., contour section 2435). The inner surface contour (e.g., surface contour 2555) may comprise a rounded portion at the second position 2570. The contoured portion (e.g., contour section 2435) may comprise a first inner radius at the first position 2565 and a second inner radius at the second position 2570, with the second inner radius being at most one fifth smaller than the first inner radius, and with the second position 2570 being located between the third position 2585 and the first position 2565.

The inlet tube 2401 or any other inlet tube described herein may have additional features or modifications, such as those described in PCT Publication No. WO 2020/016438, filed Jul. 19, 2019, titled FEED LINE FOR A PUMP UNIT OF A CARDIAC ASSISTANCE SYSTEM, CARDIAC ASSISTANCE SYSTEM AND METHOD FOR PRODUCING A FEED LINE FOR A PUMP UNIT OF A CARDIAC ASSISTANCE SYSTEM, and/or described in U.S. patent application Ser. No. 17/261,335, field Jul. 19, 2021, titled FEED LINE FOR A PUMP UNIT OF A CARDIAC ASSISTANCE SYSTEM, CARDIAC ASSISTANCE SYSTEM AND METHOD FOR PRODUCING A FEED LINE FOR A PUMP UNIT OF A CARDIAC ASSISTANCE SYSTEM, the entire contents of each of which is hereby incorporated by reference herein in its entirety for all purposes and forms a part of this specification.

Any of the embodiments of the MCS systems and pumps described herein may include an insertion tool. Various example embodiments of an insertion may be used and are described herein.

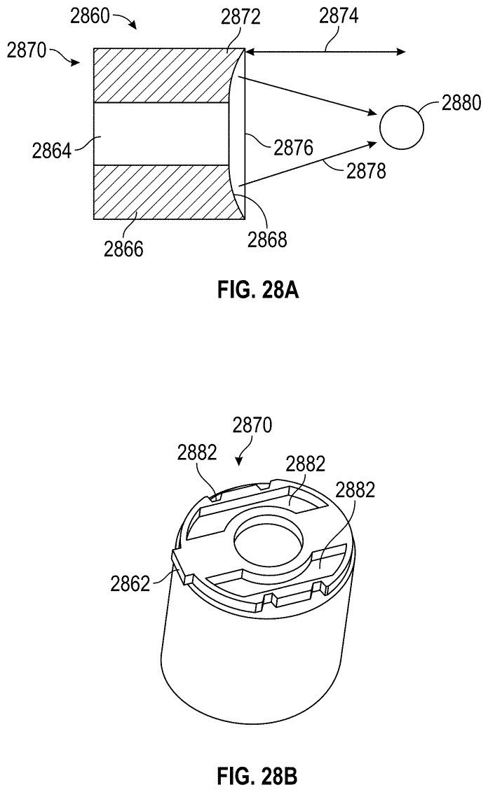

FIG. 26A-E are various views of an embodiment of an insertion tool 2632. FIG. 26A is a side view of the insertion tool 2632, FIG. 26B is a longitudinal cross-section view of the insertion tool 2632 as taken along the line A-A in FIG. 26A, FIGS. 26C and 26D are cross-section views as taken along the lines B-B and C-C respectively as indicated in FIGS. 26A and 26B, and FIG. 26E is an exploded view of the insertion tool 2632. The insertion tool 2632 may have the same or similar features and/or functions as the insertion tool 32 of FIG. 4, and vice versa. Thus, the insertion tool 2632 may be used with the pump 22, or any other pump described herein, etc.

The insertion tool 2632 may have a generally elongate tubular configuration defining a longitudinal axis 2650. As shown in FIG. 26A, the insertion tool 2632 may comprise a tubular body 2636, which may be a cylindrical tube, at a distal end. The insertion tool 2632 may comprise a hub 2634 at a proximal end. The hub 2634 may include a connector 2639 (also referred to herein as a first engagement structure), a first housing section 2638, a second housing section 2640, a cap 2637, and/or a plug 2635. The connector 2639 may include tubing 2644 with a valve 2645 (shown in FIG. 26E). As further shown in the cross sectional view of FIG. 26B, the insertion tool 2632 may also include a locking mechanism 2641, a locking pad 2642, a hemostatic valve 2649, and/or one or more sealing elements 2643. The locking mechanism 2641 may comprise locking tabs 2646 as further described below.

The tubular body 2636 at the distal end of the insertion tool 2632 may have a distal end and a proximal end with a lumen extending therebetween. The tubular body 2636 may be cylindrical. The tubular body 2636 may be made of polymer, plastic, other suitable materials, or combinations thereof. The tubular body 2636 may be made of a transparent polymer such as nylon, Grilamid®, Pebax®, which may facilitate visual confirmation of the passage of a guidewire 100 through a guidewire guide tube 83 contained in the tubular body 2636. The tubular body 2636 may be expandable. The distal end of the tubular body 2636 may comprise a taper, for example a conical portion that reduces in diameter in the distal direction, to facilitate insertion of the insertion tool 2632 (such as insertion into an introducer sheath as described herein). The distal end of the tubular body 2636, such as the tapered distal end, may removably fit into the proximal opening 90 of the guidewire aid 38. The tapered end may be a material such as 55D Pebax® molded to the tubular body. The tubular body 2636 may connect at its proximal end to a distal end of the connector 2639. The connector 2639 may connect at its proximal end to a distal end of the first housing section 2638. The first housing section 2638 may connect (e.g., rotatably, rotatable between an open position and a locked position which may be switched back and forth by rotating the second housing section 90 degrees with respect to the first housing section) at its proximal end to a distal end of the second housing section 2640. The second housing section 2640 may connect at its proximal end to a distal end of the cap 2637. A distal end of the plug 2635 may connect through a proximal end of the cap 2637.

The locking mechanism 2641 may have a longitudinally extending lumen through its body with a recess 2651 configured to accept the locking pad 2642. The locking pad 2642 may be an elastomeric material with soft durometer such as a thermoplastic elastomer, soft Pebax® or silicone. When inserted in the recess 2651, the locking pad 2642 may have an inner surface that substantially coincides with an inner surface of the longitudinally extending lumen of the locking mechanism 2641. As shown in FIG. 26B, the locking mechanism 2641 may be disposed within the hub 2634 comprising the connector 2639, the first housing section 2638, the second housing section 2640, and the cap 2637, such that all share the common longitudinal axis 2650 and the lumen of the locking mechanism 2641 is concentric with the lumen of the tubular body 2636 at least in the unlocked configuration. The locking mechanism 2641 may connect at its distal end to the proximal end of the connector 2639, and may connect at its proximal end to the distal end of the plug 2635. The plug 2635 may have a longitudinally extending lumen through its body from its distal end to its proximal end.

When connected, the plug 2635, the locking mechanism 2641, the connector 2639, and the tubular body 2636 may create a fluidically sealed pathway extending along the longitudinal axis 2650 of the insertion tool 2632. The pathway may be fluidly sealed with the pump and catheter shaft inserted therein. The valve 2649, and/or one or more sealing elements 2643 such as O-rings, may aid in creating the fluidically sealed pathway. For example, the connection between the proximal end of the connector 2639 and distal end of the locking mechanism 2641 may comprise the valve 2649. The valve 2649 may have a conical flap that reduces in width in the distal direction. When the pump or catheter shaft are inserted through the valve 2649, the conical sidewalls may expand to allow the components therethrough but stay compressed about the components to create the seal. The connection between the proximal end of the locking mechanism 2641 and the distal end of the plug 2635 may comprise one of the sealing elements 2643. The proximal end of the plug 2635 may comprise one of the sealing elements 2643 to fluidly connect to other components of the circulatory support system, such as a distal connector of the sterile sleeve 26, which may have a mating feature that locks to the plug 2635 such as by rotating projections on the plug into slots in the mating feature. The sealing elements 2643 may be O-rings or other rounded sealing elements that may sealingly engage components passing therethrough.

The fluidically sealed pathway along the longitudinal axis 2650 of the insertion tool 2632 may be configured to axially movably receive a circulatory support device or pump, such as any of the devices or pumps described herein. For example, the lumen 2620 of the tubular body 2636 may be configured to axially movably receive the pump 22 and optionally a guidewire guide tube 83, and the longitudinally extending lumen in the hub 2634 may be sized to slidably receive the shaft 16 of the MCS device (e.g., an 8 French shaft). When the pump 22 is contained in the lumen of the tubular body 2636, the shaft 16 is contained in the longitudinally extending lumen in the hub 2634, and the locking mechanism is in an unlocked state (as shown in FIG. 26C) the pump 22 may be advanced distally out of the tubular body 2636, for example into the tubular body 116 of the introducer sheath 112 before advancing from the introducer sheath into the patient's vasculature by advancing the shaft 16 in a distal direction. The tubular body 2636 of the insertion tool 2632, with a circulatory support device such as the pump 22 therein, may be configured to be received by an introducer sheath (e.g., introducer sheath 112) as described herein. As such, the tubular body 2636 of the insertion tool 2632 may have sufficient collapse resistance to maintain patency when passed through the hemostatic valves of the introducer sheath.

The insertion tool 2632 may be configured to releasably lock with the circulatory support device when inserted into the insertion tool 2632. In some embodiments, the insertion tool 2632 may releasably lock with the MSC shaft 16 (also referred to as catheter or catheter shaft) of the circulatory support device. When the insertion tool 2632 is locked with the circulatory support device, axial (e.g., longitudinal/proximal/distal) movement of the circulatory support device may be prevented. The insertion tool 2632 may lock to the circulatory support device by engagement of the locking pad 2642 with at least a portion of the circulatory support device. To engage the locking pad 2642 with the at least a portion of the circulatory support device such as the shaft 16, the locking pad 2642 may be compressed by the locking mechanism 2641.

The locking mechanism 2641 may compress the locking pad 2642 by interaction between one or more locking tabs 2646 of the locking mechanism 2641 and an inner surface or surfaces of the second housing section 2640. The locking tabs 2646 may extend radially outwardly from opposing sidewalls 2647 of the locking mechanism. The locking tabs 2646 may be offset along the longitudinal axis 2650. The second housing section 2640 along with the cap 2643 may be configured to rotate relative to the first housing section 2638, the locking tabs 2646, and the plug 2635 (with an axis of rotation being along the longitudinal axis 2650 of the insertion tool 2632). Configured this way, when the second housing section 2640 is rotated, one or more inner surfaces or sidewalls 2640B of the second housing section 2640 may contact one or more of the locking tabs 2646, causing the locking tabs 2646 to compress inwards leading to radially inward compression of the locking pad 2642. As shown in FIG. 26C, if the second housing section 2640 is rotated 90 degrees counterclockwise (as oriented in FIG. 26C, or clockwise with respect to the first housing section 2638), the inner surface sidewalls 2640B of the second housing section 2640 may contact the locking tabs 2646 (which in this embodiment are shown to have a curved outer surface) and force them inward, compressing the locking mechanism 2641 inward against the locking pad 2642. In cases where the locking tabs 2646 are offset longitudinally, the inward compression of the locking tabs 2646 and thus the locking pad 2642 against, for example, the shaft 16 may cause the shaft 16 to bend slightly in the region of the locking pad 2642, holding the shaft 16 in place. Alternatively or in addition, the shaft 16 may be compressed by the locking pad 2642 and hold/lock the shaft 16 in place.

As shown in FIG. 26C, the second housing section 2640 may include two opposing first sidewalls 2640A, which may be rounded as shown, connected by two opposing second sidewalls 2640B, which may be straight. A first distance between the two opposing first sidewalls 2640A, for example a first diameter, may be greater than a second distance between the two opposing second sidewalls 2640B, for example a second diameter. In the unlocked state, as shown in FIG. 26C, the two opposing first sidewalls 2640A may be adjacent respective locking tabs 2646. When rotated into the locked position, the two opposing second sidewalls 2640B may contact and compress respective locking tabs 2646, as described, due to the shorter distance between the second sidewalls 2640B. The locking tabs 2646 may each comprise a rounded outer corner 2646A that is contacted by a respective second sidewall 2640B, for a gradual compression and to reduce the risk of breaking the tabs. As the second housing section 2640 is turned farther counterclockwise as oriented (i.e., clockwise with respect to the first housing section 2638, the locking tabs 2646 may each comprise radially outer edges 2646B that are contacted by a respective second sidewall 2640B. The edges 2646B may be straight as shown, or otherwise match the contour of the inner surface of the second sidewall 2640B. With two opposing straight surfaces, for example, of the edges 2646B and the second sidewall 2640B in contact, the second housing section 2640 may be rotationally stationary without needing an external force by a user. Movement into engagement of the edges 2646B with the inner surface of the second sidewall 2640B may create a snap-like haptic feedback.

To unlock the circulatory support device from the insertion tool 2632, the second housing section 2640 may be rotated in the opposite direction (clockwise as oriented in FIG. 26C, or counterclockwise with respect to the first housing section 2638). The first housing section 2638 and the second housing section 2640 may comprise features that can keep the insertion tool 2632 in the unlocked position until a user of the system chooses to lock the circulatory support device in place relative to the insertion tool 2632. In some embodiments, interaction between the flexible tabs 2649 and the second housing section 2640 may keep the insertion tool 2632 in the unlocked position until a user of the system chooses to lock the circulatory support device relative to the insertion tool 2632. Likewise, the first housing section 2638 and the second housing section 2640 may comprise features that can keep the insertion tool 2632 in the locked position, as described, until a user of the system chooses to unlock the circulatory support device relative to the insertion tool 2632. In some embodiments, the interaction between the locking tabs 2646 and the second housing section 2640 may keep the insertion tool 2632 in the locked position until a user of the system chooses to unlock the circulatory support device relative to the insertion tool 2632.

The connector 2639 of the insertion tool 2632 may be configured to engage with (e.g., releasably lock/unlock with) an introducer sheath as described herein. For example, the outer surface of the distal end of the connector 2639 may comprise an inward circumferential groove that can be used to engage with a component such as mating bumps or flexible tabs in a locking cap 2924 in a proximal end port 2942 of the introducer sheath hub and/or lock of the introducer sheath. Engagement of the distal end of the connector 2639 with the locking cap 2924 may create a snap-like haptic feedback. The connector 2639 may mate with the introducer sheath locking cap 2924 in a manner that prevents rotation of the insertion tool connector 2639 with respect to the introducer hub 2922, preventing rotation of the first housing section 2638 with respect to the introducer hub when connected. For example, the distal end of the connector 2639 and the proximal end port 2942 of the introducer sheath may be oval or square or a non-circular shape. This may facilitate handling by allowing a user to hold the introducer hub 2922 and/or the first housing section 2638 with one hand while rotating the second housing section 2640 with the other hand.

FIG. 26D shows part of the connections between the connector 2639 and a distal end of the locking mechanism 2641, and the connector 2639 and a proximal end of the elongate tubular body 2636. Also shown is the tubing 2644 that may, in some embodiments, fluidly connect to the longitudinal lumen of the insertion tool 2632. The locking mechanism 2641 may include projections extending radially outwardly that are received into corresponding grooves or recesses of the connector 2639. This engagement may rotationally stabilize the locking mechanism 2641 with respect to the connector 2639. Adhesive may be added to adhere the projections and the grooves to firmly connect the connector 2639 and the locking mechanism 2641. Adhesive may be added to adhere the locking mechanism 2641 to the first housing section 2638 to firmly connect them as well. The connector 2639 may have an inward flange that has a lumen having the same size and sharing the axis 2650 with the longitudinally extending lumen in the hub 2634, which may provide a stop when inserting the tubular body 2636 into the connector 2639 during manufacturing which may protect the valve 2649 and keep the opening to the tube 2644 patent.

FIG. 26E shows an exploded view of the insertion tool 2632 according to FIGS. 26A-D and to some embodiments. As shown, the tube 2644 that may be fluidly connected to the longitudinal lumen of the insertion tool 2632 and may have a valve 2645, such as a stopcock, at its opposite end. The valve 2645 may be adjusted to prevent or allow fluid flow through the valve 2645.

The insertion tool 2632 may have a length within the range of from about 85 mm to about 200 mm (e.g., about 192 mm). In some embodiments, the longitudinal lumen of the insertion tool 2632 may comprise a diameter within the range of from about 4.5 mm to about 8.0 mm (e.g., about 5.55 mm). The insertion tool 2632 may be sized and configured such that the marking 37 (see FIG. 7) is revealed proximal to the insertion tool hub 2634 when the pump 22 is fully within the tubular body 2636. The insertion tool 2632 may include a hemostasis valve (e.g., hemostatis valve 2645) to seal around the circulatory support system passing therethrough (e.g., to seal around the MCS shaft 16). If provided, the hemostasis valve may accommodate passage of the larger diameter MCS device which includes the pump. In a commercial embodiment of the circulatory support system, the MCS device as packaged is pre-positioned within the insertion tool 2632 and a guidewire aid is pre-loaded within the MCS device and shaft 16, as described herein.

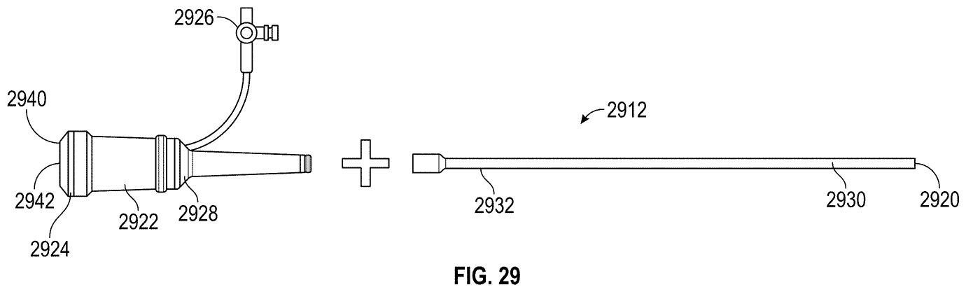

FIG. 27 is a partial cross sectional view, through an impeller and magnetic coupling region, of an embodiment of a rotor bearing system 2700 of a pump that may be used with the various MCS systems described herein. The rotor bearing system 2700 may have a contactless torque transfer, and radial and axial motor mount, that is shown as an exemplary embodiment in the form of a pump for cardio-vascular support.

The rotor bearing system 2700 has a housing 2780. The housing 2780 may be a motor housing that encapsulates a motor, drive shaft and/or a drive magnet, which may be hermetically sealed from the surrounding environment. Within the housing 2780 a first cylindrical permanent magnet 2730 is seated on a shaft 2706 driven by a motor, not shown, and said permanent magnet 2730 is mounted to rotate about a first axis 2705.

The housing 2780 may have a first cylindrical portion having a first outer diameter 2731 (e.g., in a range of 5 to 7 mm, preferably 6 mm) that radially encompasses the motor, a second cylindrical portion having a second outer diameter 2732 that is less than the first outer diameter (e.g., less than the first outer diameter by a range of 0.3 to 1 mm, preferably by 0.5 mm), and a third cylindrical portion having a third outer diameter 2733 that is less than the second outer diameter (e.g., less than the second outer diameter by 1.7 to 2.3 mm, preferably by 2.0 mm).

The second outer diameter 2732 may securely mate with an inlet tube housing 2722, wherein the second outer diameter and the inlet tube housing 2722 are sized so the outer diameter of the inlet tube housing is flush with the first outer diameter 2731 (e.g., the thickness of the inlet tube housing 2722 may be equal to the difference between the first outer diameter and second outer diameter divided by 2. The third outer diameter 2733 of the housing 2780 may be for example in a range of 3.2 to 3.8 mm, preferably 3.5 mm.

The rotor bearing system 2700 may further comprise a rotor 2770 for conveying a liquid, wherein the rotor 2770 comprises a second permanent magnet 2740 in the form of a hollow cylinder that is also mounted to rotate about the first axis 2705. The second permanent magnet 2740 in the form of a hollow cylinder is arranged in a part 2772 in the form of a hollow cylinder of the rotor 2770. The second permanent magnet 2740 in the form of a hollow cylinder optionally comprises a back-iron 2750 on its exterior.

In some embodiments the first permanent magnet 2730 may have an outer diameter of 3 mm, a magnet height of 1 mm, and a length of 3.2 mm (e.g., in a range of 3 to 4.2 mm). The second permanent magnet 2740 may have an outer diameter of 5.3 mm (e.g., in a range of 5 to 5.3 mm), a magnet height of 0.6 mm (e.g., in a range of 0.5 to 0.6 mm), and a length of 3.2 mm (e.g., in a range of 3 to 4.2 mm). The stagger 2715 may be 1 mm (e.g., in a range of 0.1 to 1.2 mm). The rotor 2770 may have an outer diameter of 5.3 mm (e.g., less than the second outer diameter 2732 by a range of 0.1 to 0.4, preferably 0.2 mm) and a length of 15 mm.

The rotor 2770 may be arranged as an impeller that converts the mechanical power transferred by the coupling (e.g., magnetic coupling) into hydraulic power to convey a blood flow against a blood pressure. The rotor 2770 may further comprise a tapered or conical part 2771 that is mated to the part 2772 in the form of a hollow cylinder. The outer circumference of the base surface of the conical part 2771 may be connected with the ring-shaped opening on an axial end of the part 2772 in the form of a hollow cylinder.

The first permanent magnet 2730 and the second permanent magnet 2740 may at least partially axially overlap in the axial area labeled by the reference symbol 2716. The first permanent magnet 2730 is in this case is arranged axially staggered in relation to the second permanent magnet 2740. The centers of the first permanent magnet 2730 and the second permanent magnet 2740 are marked by vertical lines, wherein the axial stagger 2715 is drawn between these two vertical lines.

Due to the axial stagger 2715, the second permanent magnet 2740 may be subjected to a force directed to the right in FIG. 27, so that a ball 2717 that is arranged in the rotor 2770 is pushed onto a cone 2718 arranged in the housing 2780, so that a first bearing 2720 and a third bearing 2790, which in this case form a combined axial and radial bearing 2719, are held in contact. Alternatively, a ball may be arranged in the housing 2780 and a cone arranged in the rotor. When used as intended, the ball 2717 rotates in the cone 2718, so that both radial and also axial forces can be absorbed. The combined axial and radial bearing 2719 is in this case a solid body bearing. The ball 2717 is arranged in the conical part 2771. The axial and radial bearing function is achieved by the combination of the two elements ball 2717 and cone 2718. The ball 2717 for example, may have a diameter in a range of 0.5 mm to 0.9 mm, preferably 0.7 mm, and the cone 2718 may have a diameter of 1 mm, a height of 0.8 mm, and a cone angle within a range of 70° to 90°, preferably 80°. The axial bearing function of the combined bearing 2719 has the function of the first bearing and is designed for the relative axial positioning of the rotor 2770 and the housing 2780 and/or the shaft 2706 to each other and to absorb an axial force resulting by the arrangement of the first permanent magnet 2730 and the second permanent magnet 2740. Moreover, the axial force on the rotor bearing system 2700 may be adjusted, so that the exerted force settings can be optimized.

The region of the housing 2780 that comprises the first permanent magnet 2730, may be at least in part radially surrounded by part 2772 in the form of a hollow cylinder of the rotor 2770. A channel 2774 in the form of a hollow cylinder may then be formed between the housing 2780 and part 2772 of the rotor 2770, through which a liquid can flow. Bores or perforations 2702 may be arranged in the rotor 2770, preferably in the conical part 2771 of the rotor 2770, or in a transition of the conical part 2771 to the part 2772 in the form of a hollow cylinder of the rotor 2770 and may be in fluid communication with the channel 2774. In use, when the rotor 2770 spins liquid may be centrifugally expelled from the bores 2702 and liquid may be pulled into the channel 2774 to replace the expelled liquid in a continuous flow. Flow arrow 2711 in this case indicates the direction of flow of the liquid through the gap 2774. Flow arrow 2712 indicates the direction of flow of liquid transferred by the rotor vanes 2773.

A second bearing 2710, which can be arranged as a radial, hydrodynamic, and blood-lubricated plain bearing, may be arranged on the end of the conical part 2771 of the rotor 2770 facing away from the housing 2780. The second bearing 2710 may be designed to absorb radial forces and to position the axis of rotation of the second permanent magnet 2740 in alignment with the axis of rotation 2705 of the shaft 2706 or the first permanent magnet 2730. In this case, the second bearing 2710 may be arranged between the rotor 2770 and an insert 2721, which can be fastened, in particular clamped in or pressed in, in a ring-shaped end on a second housing 2722, which is in turn fastened onto the housing 2780. The second housing 2722 in this case may form an exterior skin of the rotor bearing system 2700, wherein the second housing 2722, which can also be called an impeller housing, has a plurality of outlet windows 2723. The insert 2721 is preferably a bearing housing or star that can be firmly attached (e.g., glued, welded, or friction fitted) to the second housing 2722. The bearing star 2721 may have an outer diameter of 6 mm (e.g., in a range of 5 to 7 mm) and a length of 3 mm (e.g., in a range of 2 to 5 mm). The second housing 2722 may have an outer diameter of 6 mm (e.g., in a range of 5 to 7 mm), a length of 18 mm (e.g., in a range of 15 to 21 mm), and a wall thickness of 0.25 mm (e.g., in a range of 0.15 to 0.5 mm).

Alternatively, the insert 2721 and second housing 2722 may be manufactured as a single piece, which may have a consistent inner diameter. In this arrangement an extended inlet cannula may be connected to the combined insert and second housing 2722 for example by laser welding.

The bearing 2710 may have a diameter of 1 mm (e.g., in a range of 0.75 to 1.5 mm) and a length of 1 mm (e.g., in a range of 0.75 to 2 mm).

Due to the axial stagger 2715 determined by the design between the first permanent magnet 2730 and the second permanent magnet 2740, a defined axial force in the exemplary embodiment in FIG. 27 acts on the rotor 2770 in the direction of the motor, that is to say from left to right in the exemplary embodiment in FIG. 27. This force is opposed by a hydraulic force imposed on the rotor 2770 during operation, that is to say from right to left in the exemplary embodiment in FIG. 27, which is in the opposite direction of liquid flow 2711 generated by the spinning rotor vanes 2773.

In the present case, the axial force originating from the coupling of the first permanent magnet 2730 and the second permanent magnet 2740 may be optimized to be larger than the maximum expected hydraulic force, which ensures that the rotor 2770 is at all times held in a defined axial position, without being too much larger than the maximum expected hydraulic force, which may allow the combined axial and radial bearing 2719 to not be unnecessarily overloaded, thus minimizing friction and wear as well as reduction of torque transmitted to the rotor. This axial force may be optimized by adjusting the dimensions (e.g., length, thickness, outer diameter) of both permanent magnets 2730, 2740, and the axial displacement or stagger distance 2715, and the segment angle, a, if a Halbach configuration is implemented.

Optimization studies were done by the applicant using a Halbach magnet configuration, with a segment angle α of 45° and a pump device having an outer diameter of 6.2 mm. Due to the diameter constraints of the device the inner and outer diameter of the first permanent magnet was chosen to be 1.0 mm and 3.0 mm respectively. The inner and outer diameter of the second permanent magnet was chosen to be 4.1 mm and 5.3 mm respectively. The length of each magnet and the stagger 2715 were modified to study the effect on axial force and torque and optimized. The sum of the magnet length and stagger was limited to 4.2 mm due to a constraint on length of rigid section of the pump so it can traverse tortuous vascular pathway during endovascular delivery to the heart. Conclusions of the study found that an optimized design has a magnet length (length of both permanent magnets 2730, 2740) of 3.2 mm and an axial displacement or stagger 2715 of 1.0 mm generated to best results. A stagger 2715 in a range of 0.5 to 1 mm may be the basis for alternative embodiments but were found to be less optimal. These results may represent an optimized coupling configuration for the devices tested. Because the forces applied to the impeller and coupling are a function of overall device diameter, inlet tube length, impeller design, maximum impeller speed or blood flow rate, and other features or dimensions that affect hydraulic force, bearing frictional losses, and eddy current losses the results may differ with devices having different dimensions or features compared to the ones tested.

For the purposes of this study a maximum fluid load was assumed to be 1.2 mNm, frictional loss of the bearings was assumed to be 0.2 mNm, and eddy current loss was assumed to be 0.1 mNm for a total load torque of 1.5 mNm during normal operation. A safety factor of 3 was used making the maximum load torque 4.5 mNm. The friction and wear behavior can also be optimized by enlarging the cone angle of the cone 2718, wherein sufficient radial load capacity must be ensured.

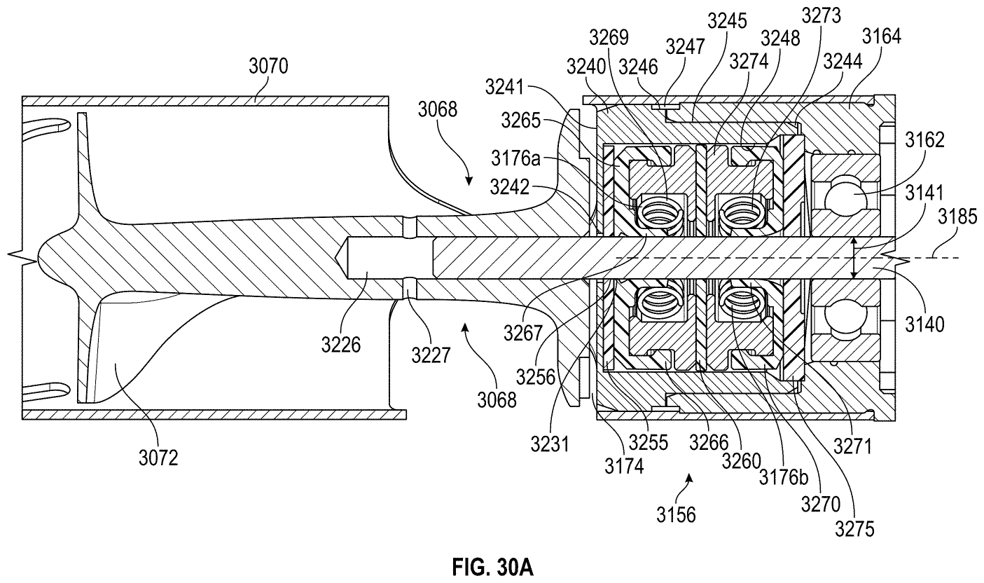

FIGS. 28A-B show aspects of an ultrasound transducer 2860 that may be incorporated in the embodiments of a circulatory support system described herein. For example, the ultrasound transducer 2860 or features thereof may be incorporated into embodiments of the MCS system and pump described herein for longer-term use, such as for therapy of cardiogenic shock, and/or in embodiments having magnetic drives with a sealed off motor housing, and/or in other embodiments.

The ultrasound transducer 2860 may be provided distally of the blood intake port (also referred to as the pump inlet and/or inlet opening herein). The ultrasound transducer 2860 can include a positioning tab 2862 configured to couple with a positioning channel of the nose piece (also referred to as distal tip herein) 64. A guide wire lumen (also referred to as a guidewire port herein) 76 can extend through the ultrasound transducer 2860. The ultrasound transducer 2860 may comprise an acoustical backing 2866, having a proximal concave surface 2868 and a distal end surface 2870. The guide wire lumen 76 may extend through the acoustical backing 2866. The proximal concave surface 2868 may be provided with at least one and preferably two or more piezo elements 2872, focused for convergence at a focal distance 2874 within the range of from about 6 mm to about 14 mm and preferably approximately 10 mm from the concave surface 2868. The piezo elements 2872 on the concave surface 2868 can direct ultrasonic waves 2878 to a focus region 2880 positioned at the focal distance 2874. Concave surface 2868 and piezo elements 2872 may be covered by an acoustical impedance matching layer 2876.

The distal end 2870 of the ultrasound transducer 2860 may be provided with a plurality of electrodes 2882, to connect conductors to the piezo elements 2872. In addition, a positioning structure such as a tab or recess, such as for example, the positioning tab 2862, may be provided to ensure appropriate rotational orientation of the ultrasound transducer 2860 by engaging a complementary tab or recess, such as the positioning channel mentioned above, in the adjacent structure such as the nose piece 64 or MCS/VSD inlet tube 70. The focus region 2880 of the directed ultrasound waves 2878 is therefore positioned in the blood flow path adjacent the blood intake ports or downstream of the blood intake ports within the blood flow channel, to provide blood flow velocity data by assessing Doppler shift of the reflected ultrasound waves detected by the ultrasound transducer 2860.

Other embodiments or features of an ultrasound flow sensor and methods for measuring flow by ultrasound may be incorporated into the MCS system or pump, such as those described in PCT Pub. No. WO 2020/064707, filed Sep. 24, 2019, titled METHOD AND SYSTEM FOR DETERMINING A FLOW SPEED OF A FLUID FLOWING THROUGH AN IMPLANTED, VASCULAR ASSISTANCE SYSTEM, in U.S. application Ser. No. 17/274,354, filed Mar. 8, 2021, titled METHOD AND SYSTEM FOR DETERMINING A FLOW SPEED OF A FLUID FLOWING THROUGH AN IMPLANTED, VASCULAR ASSISTANCE SYSTEM, in PCT Pub. No. WO 2019/234166, filed Jun. 6, 2019, titled METHOD FOR DETERMINING A FLOW SPEED OF A FLUID FLOWING THROUGH AN IMPLANTED, VASCULAR ASSISTANCE SYSTEM AND IMPLANTABLE, VASCULAR ASSISTANCE SYSTEM, and/or in U.S. patent application Ser. No. 15/734,523, filed Dec. 2, 2020, titled SYSTEMS AND METHODS FOR DETERMINING A FLOW SPEED OF A FLUID FLOWING THROUGH A CARDIAC ASSIST DEVICE, the entire contents of each of which is incorporated by reference herein in its entirety for all purposes and forms a part of this specification.

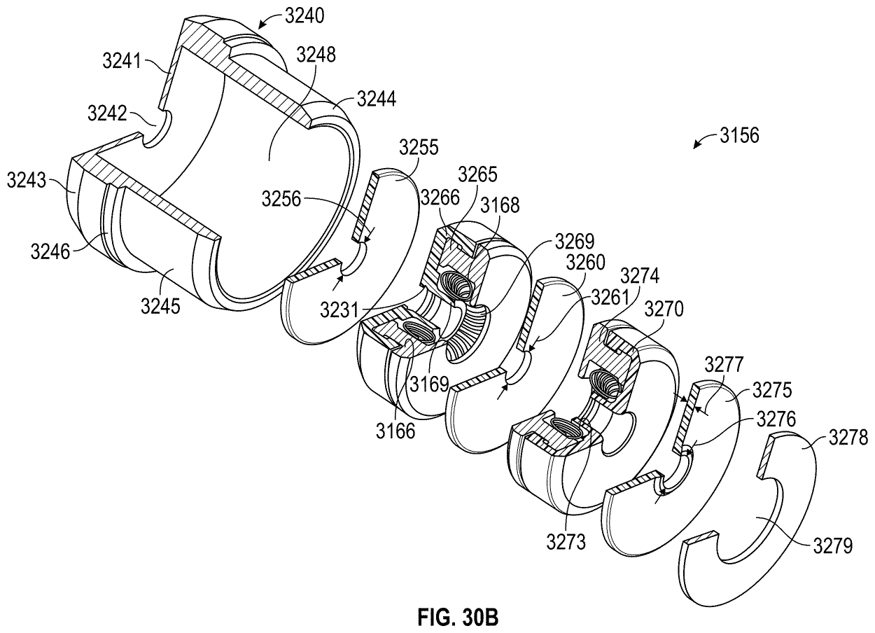

FIG. 29 illustrates a side elevational view of an expandable introducer sheath 2912. The expandable introducer sheath 2912 may be used with any of the embodiments of the MCS system or pump described herein. The expandable introducer sheath 2912 may have a hub 2922 and associated components similar to the introducer sheath 112 described in connection with FIG. 5, and vice versa. Further, the elongate tubular body of the introducer sheath 2912 may be expandable from a first reduced inside cross-sectional area to a second, enlarged inside cross-sectional area, such as to permit passage of a device having an outer diameter (OD) greater than the first reduced cross-sectional area. The introducer sheath may be biased to return to or approximately to the first reduced cross-sectional area following expansion in response to passage of a sheath enlarging device there through (e.g., the MCS and/or VSD devices described herein). The expandable introducer sheath 2912 may include an expandable support structure 2932 such as a tubular framework of a plurality of zigzag segments of a shape memory material such as Nitinol which permit radial expansion in the presence of an enlarging device passing therethrough, but will return to the first reduced cross-sectional area following removal of the device. The expandable support structure 2932 may be enclosed within a tubular flexible membrane 2930, which can accommodate radial expansion and contraction. Further as shown, the expandable introducer sheath 2912 may include a distal end 2920, a proximal end 2940, a side port 2926, suture eyelet(s)/eye 2928, a proximal hub 2922, and a proximal end port 2942 similar to the distal end 120, proximal end 118, side port 126, suture eyelet(s)/eye 128, proximal hub 122, and proximal end port 124 of the introducer sheath 112 described herein. The expandable introducer sheath 2912 may also include a locking cap 2924 at its proximal end with one or more features that can engage/lock with an insertion tool (e.g., insertion tool 32 and/or insertion tool 2632) such as a connector 2639 and/or a dilator (e.g., dilator 114) as described herein.

Another embodiment of an MCS device having a sealed rotary shaft is shown in FIGS. 30A-30C. FIG. 30A is a partial cross-sectional view of the device having two lip seals facing one another, a front disc, a middle disc, and a rear disc contained in a seal housing, FIG. 30B is an isometric, exploded, partially cut-away view thereof, and FIG. 30C is a cross-sectional view of the seal components shown isolated as a subassembly. The MCS device of FIGS. 30A-30C, or variations or embodiments thereof, may be included in any of the MCS systems described herein and may include any of the features for MCS devices described herein, and vice versa. Thus, for example, the pump 22, the MCS system 10, the motor housing 74, the pump 1900, the pump 2062, and/or the pump region 2160, etc. may include the MCS device or features thereof of FIGS. 30A-30C, in particular the sealing features thereof. Alternatively or in addition, any of the pump embodiments described herein may include other seal features, for example as described in U.S. Provisional Application No. 63/229,436, titled SEAL FOR A MECHANICAL CIRCULATORY SUPPORT DEVICE and filed on Aug. 4, 2021, the entire content of which is incorporated by reference herein for all purposes and forms a part of this specification.

As shown in FIGS. 30A-30C, the device includes a distal annular radial or rotary shaft seal 3266 having a radially inward contact lip 3267 forming a seal cavity 3176a. The contact lip 3267 and seal cavity 3176a of the distal seal 3266 face proximally. The distal seal 3266 thus has an “open side” facing proximally toward the motor, and a “flat side” facing distally toward the impeller and blood. The distal seal 3266 is thus oriented “backwards” from conventional orientations. In some embodiments, the “open side” may be a side of the seal 3266 formed in part by upper and/or lower flanges or lips of the seal 3266. A cavity may be formed by the open side of the seal 3266. The cavity may be formed between an end wall of the seal 3266 and the one or more flanges or lips of the seal 3266. The cavity may have a spring and/or grease located therein. Further details of the end wall, lips, etc. are described herein.

The device further includes a proximal annular radial or rotary shaft seal 3270, having a radially inward contact lip 3271 forming a seal cavity 3176b. The contact lip 3271 and a seal cavity 3176b of the proximal annular seal 3270 faces distally. The proximal seal 3270 thus has an “open side” (as described above) facing distally toward the motor, and a “flat side” facing proximally toward the impeller and blood. Therefore, the seal assembly includes the proximal annular seal 3270 and the distal annular seal 3266 having opposite orientations, with their contact lips 3267, 3271 and seal cavities 3176a, 3176b facing one another.

The lips 3267, 3271 contact the shaft 3140. The lips 3267, 3271 may extend along the shaft 3140. All or a part of the radially inward surface or surfaces of the lips 3267, 3271 may contact the shaft 3140. The lips 3267, 3271 may be flat, and/or have non-flat features, as described in further detail herein, for example with respect to FIG. 30C.

The seals 3266, 3270 may include radially outer lips 3263, 3264. The lips 3263, 3264 may contact a radially inward surface of the housing or other component of the seal compartment. The lips 3263, 3264 may extend along the housing or other component. The lips 3263, 3264 may seal off the space between the seal 3266, 3270 and the housing or other component. The radially outer surfaces of the lips 3263, 3264 may be flat, non-flat, or combinations thereof.

The lips 3263, 3264 may extend from respective end walls 3262, 3259. The lip 3263 extends distally from the end wall 3262. The lip 3264 extends proximally from the end wall 3259. The end walls 3262, 3259 may refer to the “flat” sides described herein. The radially inner lips 3267, 3271 may extend from the end walls 3262, 3259, as described. The outer lips 3263, 3264 may extend perpendicular to the end walls 3262, 3259, either under no external forces and/or when installed in the seal compartment. The outer lips 3263, 3264 may have the same or similar features as the inner lips 3267, 3271, such as the leading edge, groove or recess, etc.

In some embodiments, a middle elastomeric disc 3260 may be positioned between the proximal annular seal 3270 and the distal annular seal 3266. A distal elastomeric disc 3255 may be positioned distal to the distal annular seal 3266. A proximal elastomeric disc 3275 may be positioned proximal to the proximal annular seal 3270.

Optionally, a seal housing made of a front seal container 3240 and an optional seal container cap 3278 (see FIGS. 30B and 30C), may contain the seal components in a subassembly. The subassembly may be inserted over the drive shaft 3140 and into a motor housing 3164. Alternatively, the seal components may be assembled in the motor housing by inserting the components separately and sequentially over the drive shaft 3140 into a cavity in the motor housing. The seal components may then be covered with a rear (proximal) seal cap 3278 that may be attached (e.g., welded, friction fit, form fit, glued) to the motor housing.

Both the distal elastomeric disc 3255 and the middle elastomeric disc 3260 may be made from an elastomeric, biocompatible material such as PTFE, an elastic polyurethane, or a compound material such as PTFE and Polyimide. As shown in FIG. 30B, one or more of the discs 3255, 3260 may have an inner diameter (ID) 3256, 3261 that is less than the outer diameter (OD) of the drive shaft 3140, which optionally may include an impeller back extension 3154, that the inner diameter contacts. For example, the ID 3256, 3261 may be in a range of 80% to 95% (e.g., about 87%) that of the OD 3141. In one implementation, the ID 3256, 3261 is 0.52 mm+/−0.02 mm and the OD 3141 is 0.60 mm+/−0.01 mm. This dimensional difference creates high interference between the elastomeric discs 3255, 3260 and drive shaft to maintain a seal. For example, an ideal interference may be in a range of 0.070 mm to 0.080 mm. The elastomeric discs 3255, 3260 may both have a thickness in a range of 80 μm to 140 μm (e.g., about 100 μm).

The properties of the elastomeric discs 3255, 3260 such as high interference, material durometer (e.g., in a range of 70 to 85 Shore), and thickness, may allow for the disc to deform when inserted over the drive shaft. For example, the disc may compress outward such that the disc ID may stretch, or the plane of the disc may curve particularly in a region close to the ID. The deformation of the disc may provide a contact pressure with the drive shaft 3140 even as the disc material wears over time. Furthermore, the high interference provides an amount of material that may be worn down before contact pressure is reduced to zero, which may prolong the functional duration of the disc 3255, 3260 to act as a blood barrier. Furthermore, the high interference may compensate for small tolerances of eccentricity of the drive shaft within the disc.

The properties of the discs 3255, 3260 may allow them to act as a fluid barrier, at least for a portion of the intended duration that the MCS device is in use, while minimizing friction or decrease in torque transmission. Additionally, the distal elastomeric disc 3255 may function as a first barrier to blood at least for a portion of duration of use. The middle elastomeric disc 3260, may function as an additional barrier to blood if it manages to pass the more distal barriers. Also, the disc 3260 may act as a divider between the distal annular seal cavity 3176a and proximal annular seal cavity 3176b help to keep grease that is contained in these cavities next to each annular seal, which in turn prolongs the functional duration of the annular seals. Optionally, the grease or lubricant dispensed in the distal seal cavity 3176a may be the same or different than that dispensed in the proximal seal cavity 3176b. In some embodiments, the proximal disc 3276 may have the same or similar features as the distal and middle discs 3255, 3260.

Other than their relative position and orientation, the distal seal 3266 and proximal seal 3270 may have similar properties to one another or to other seals 3156 disclosed in relation to other implementations. For example, both the distal and proximal seals may have a seal holder 3265, 3274, an annular seal with a contact lip 3267, 3271, a seal cavity 3176a, 3176b, partially defined by the seal holder and annular seal, and/or a garter spring 3269, 3273 held in the respective seal cavity 3176a, 3176b. The seals 3266, 3270 may have the same inner diameter and lip dimensions. Optionally the seals 3266, 3270 may have different outer diameters primarily so they are easily distinguishable from one another during manufacturing.

Alternative to a garter spring 3269, 3273 the seals may contain a different component that applies radially inward force such as an O-ring or not have a separate component that applies the force, wherein properties of an elastomeric annular seal with a contact lip self-applies a radially inward contact force.

The distal and proximal annular seals 3266, 3270, may be made from a biocompatible elastomeric material such as PTFE, an elastic polyurethane, or a compound material such as PTFE and Polyimide, which optionally may have one or more additives to enhance durability. Grease may be contained in one or both seal cavities 3176a, 3176b, and optionally a third grease reservoir held between the proximal seal and proximal disc 3275, and may be the same grease or different greases. In one implementation a first grease is deposited in the distal seal cavity, which may have a higher viscosity and grease consistency (e.g., NLGL Class 4 or higher) than a third grease (e.g., NLGL Class 2) deposited in the proximal seal cavity or a second grease held in the third grease reservoir held between the proximal seal and proximal disc. In another implementation grease is deposited in the distal seal cavity (e.g., NLGL Class 4 or higher) and an oil is deposited in the proximal seal cavity.

Optionally, the distal seal 3266 may have a leading edge 3231 on its distal face, which in addition to the contacting lip 3267 is a surface of the distal seal that contacts rotating parts such as the drive shaft 3140. The leading edge 3231 is a portion of the distal annular seal 3266 with an inner diameter that is less than the inner diameter of a portion of the contacting lip 3267 located proximally of the leading edge 3231. The leading edge 3231 may be a portion of the distal annular seal 3266 with an inner diameter that is less than the outer diameter of the motor drive shaft 3140 that the inner diameter mates with. For example, the ID of the leading edge may be in a range of 75% to 95% (e.g., 80% to 90%, about 87%) that of the OD 3141. In one implementation the ID is 0.52 mm and the OD 3141 is 0.60 mm. By making a flush connection to the rotating shaft 3140 on the distal face of the seal, the leading edge may function to reduce the occurrence of blood getting actively drawn underneath the contacting lip 3267, which may contribute to increasing the longevity of the seal. The distal annular seal 3266 may be made as shown with a groove between the leading edge 3231 and contact lip 3267. The leading edge 3231 may be formed in part by an adjacent groove or recess formed in the inner surface of the lip 3267. Alternatively, the leading edge 3231 may have a smooth transition to the contact lip 3267.

The orientation of the proximal seal 3270, wherein the contact lip 3271 and seal cavity 3176b are directed distally, may facilitate the overall sealing function in a number of ways: for example, lubricating grease is held in the cavities 3176b and 3176a between the distal seal 3266 and proximal seal 3270 which coats the contact surface between the contact lips 3267, 3271 and the drive shaft 3140 to reduce wear, minimize reduction of torque transmission or heat formation, and resist ingress of blood; a higher pressure on the distal side of the seal 3270 relative to the proximal side (e.g., due to compressed grease held in the seal cavity 3176b or in the event that blood manages to pass through the more distal blood barriers) may support the contact pressure of the contact lip 3271. The axial length of a portion of the contact lip 3271 that contacts the shaft may be in a range of 0.3 to 0.8 mm (e.g., about 0.5 mm).

Optionally, the device may have the proximal disc 3275 positioned proximal to the proximal seal 3270 as shown in FIG. 30A. The proximal disc may function as another barrier to prevent blood from entering drive shaft bearings 3162 or the motor compartment. Furthermore, the proximal disc may help to account for small tolerances in eccentricity of the drive shaft. The proximal disc 3275 may be made from a biocompatible elastomeric material such as PTFE or an elastic polyurethane or a compound and have a generally disc shape with a center hole having an inner diameter 3276 through which the drive shaft 3140 passes and makes contact. The ID 3276 may be in a range of 80% to 97% (e.g., about 93%) that of the OD 3141. In one implementation the ID is 0.56 mm and the OD 3141 is 0.6 mm, which may be greater than the ID of the distal disc 3255 or middle disc 3260 to have less impact on torque transmission losses. Optionally, the proximal disc 3275 may have a greater thickness than the distal or middle discs 3255, 3260 as shown in FIG. 30A, which together with the elastomeric properties of the disc may provide an axial compression of the sealing components when the proximal disc is compressed between a front seal container 3240 and an edge on the motor housing 3164. For example, the thickness of the proximal, middle and distal discs may be in a range of 0.10 mm to 0.15 mm. The proximal disc 3275 may be axially compressed due to dimensions of the stack up of seal components in the axial direction and the space within the housing that compresses the stack. In some embodiments, the proximal disc 3275 may be non-flat, e.g. spherical, such as a Belleville washer shape, to provide compression.

FIGS. 30B and 30C show the device of FIG. 30A but having a relatively thinner the proximal disc 3275, as well as the addition of a seal container cap 3278. In this implementation all of the sealing components are contained within a seal container, for example as a subassembly. The seal container may include a front seal container 3240 and the seal container cap 3278, which may be both made from a metal such as stainless steel or titanium and connected securely for example, with a friction fit, form fit, threading, or weld.

The front seal container 3240 functions to contain the seal components with or without the seal container cap 3278 and facilitate manufacturing. The front seal container has a flat, rigid distal surface 3241 that provides a surface for mechanically pressing the seal components into the motor housing 3164 while protecting the softer, more fragile seal components. The flat, rigid surface 3241 also ensures the axial gap 3174 between the surface 3241 and impeller is consistent so blood in the axial gap is expelled, and the back face of the rotating impeller does not contact the seal components inadvertently. The surface 3241 has a central hole 3242, which has an inner diameter that is larger than the outer diameter of the drive shaft 3140. For example, the hole 3242 may have a diameter that is in a range of 0.080 mm to 0.150 mm (e.g., about 0.100 mm) greater than the outer diameter of rotating parts passing through the hole, which may function as a physical filter to prevent particles from escaping the container as a risk management measure. For example, the hole 3242 may be in a range of 0.68 mm to 0.75 mm (e.g., about 0.70 mm) when the drive shaft has a diameter of 0.60 mm. In other words, a radial gap between the drive shaft and the container 3240 may be in a range of 0.040 mm to 0.075 mm (e.g., about 0.050 mm). The front seal container has cylindrical side walls with an inner surface 3248 that functions to constrain the seal components ensuring there is no lateral movement, which could compromise the integrity or longevity of the seals. A proximal chamfer 3244 facilitates insertion into the motor housing during manufacturing. A distal chamfer 3243 facilitates insertion of an inlet tube 3070, or alternatively an impeller housing 3082 over the front seal container 3240. Furthermore, the front seal container 3240 may have a recessed outer surface 3245 for inserting into the motor housing 3164. An embodiment of a heart pump having a seal element 3156 as shown in FIG. 30A may have a motor housing with a length no greater 25.5 mm. With additional length added to the motor housing by the seal subassembly and an optional wiring module connected to the proximal end of the motor housing, the length of the motor housing may be extended to no more than 33 mm.

A method of manufacturing a seal subassembly may include but not be limited to inserting the seal components into the front seal container in the order and orientation described herein, dispensing grease in the seal cavities optionally sequentially or simultaneously, releasing air bubbles using a centrifuge or vacuum chamber, and closing the seal container with the seal container cap 3278. The seal subassembly may be inserted over a drive shaft 3140, optionally into a motor housing, and connected to the motor housing, for example by laser welding an intersection which may include a rabbet 3246 of the front seal container 3240 and a rabbet 3247 of the motor housing. The impeller may be connected to the drive shaft, for example with an arrangement as described herein with respect to other embodiments and figures. An impeller housing 3082 or an inlet tube 3070 having an integrated impeller housing may be connected to the motor housing and/or front seal container 3240. The device may be packaged in an airtight package with air evacuated to prevent drying of the grease dispensed in the seals.

Any embodiments of the MCS systems, and features thereof, described herein may include various additional features or modifications, such as those described, for example, in PCT Pub. No. WO 2020/089429, filed on Oct. 31, 2019, titled SYSTEM AND METHOD FOR CONTROLLING A CARDIAC ASSISTANCE SYSTEM, in U.S. patent application Ser. No. 17/290,083, filed Apr. 29, 2021, titled SYSTEM AND METHOD FOR CONTROLLING A CARDIAC ASSISTANCE SYSTEM, in PCT Pub. No. WO 2019/229221, filed on May 30, 2019, titled ELECTRONICS MODULE AND ARRANGEMENT FOR A VENTRICULAR ASSIST DEVICE, AND METHOD FOR PRODUCING A VENTRICULAR ASSIST DEVICE, in U.S. patent application Ser. No. 17/057,039, filed Nov. 19, 2020, titled ELECTRONICS MODULE AND ARRANGEMENT FOR A VENTRICULAR ASSIST DEVICE, AND METHOD FOR PRODUCING A VENTRICULAR ASSIST DEVICE, in PCT Pub. No. WO 2019/234152, filed on Jun. 6, 2019, titled DEVICE AND METHOD FOR DETERMINATION OF A CARDIAC OUTPUT FOR A CARDIAC ASSISTANCE SYSTEM, in U.S. patent application Ser. No. 15/734,841, filed Jun. 18, 2021, titled DEVICE AND METHOD FOR DETERMINATION OF A CARDIAC OUTPUT FOR A CARDIAC ASSISTANCE SYSTEM, in PCT Pub. No. 2020/0030706, filed Aug. 7, 2019, titled DEVICE AND METHOD FOR MONITORING THE STATE OF HEALTH OF A PATIENT, in U.S. application Ser. No. 17/266,056, filed Oct. 13, 2021, titled DEVICE AND METHOD FOR MONITORING THE STATE OF HEALTH OF A PATIENT, in PCT Pub. No. WO 2020/064707, filed Sep. 24, 2019, titled METHOD AND SYSTEM FOR DETERMINING A FLOW SPEED OF A FLUID FLOWING THROUGH AN IMPLANTED, VASCULAR ASSISTANCE SYSTEM, in U.S. application Ser. No. 17/274,354, filed Mar. 8, 2021, titled METHOD AND SYSTEM FOR DETERMINING A FLOW SPEED OF A FLUID FLOWING THROUGH AN IMPLANTED, VASCULAR ASSISTANCE SYSTEM, in PCT Pub. No. WO 2019/234148, filed Jun. 9, 2019, titled IMPLANTABLE VENTRICULAR ASSIST SYSTEM AND METHOD FOR OPERATING SAME, in U.S. patent application Ser. No. 15/734,342, filed Jul. 30, 2021, titled IMPLANTABLE VENTRICULAR ASSIST SYSTEM AND METHOD FOR OPERATING SAME, in PCT Pub. No. WO 2019/234149, filed Jun. 9, 2019, titled SENSOR HEAD DEVICE FOR A MINIMAL INVASIVE VENTRICULAR ASSIST DEVICE AND METHOD FOR PRODUCING SUCH A SENSOR HEAD DEVICE, in U.S. patent application Ser. No. 15/734,036, filed Jun. 8, 2021, titled SENSOR HEAD DEVICE FOR A MINIMAL INVASIVE VENTRICULAR ASSIST DEVICE AND METHOD FOR PRODUCING SUCH A SENSOR HEAD DEVICE, in PCT Pub. No. WO 2019/234166, filed Jun. 6, 2019, titled METHOD FOR DETERMINING A FLOW SPEED OF A FLUID FLOWING THROUGH AN IMPLANTED, VASCULAR ASSISTANCE SYSTEM AND IMPLANTABLE, VASCULAR ASSISTANCE SYSTEM, in U.S. patent application Ser. No. 15/734,523, filed Dec. 2, 2020, titled SYSTEMS AND METHODS FOR DETERMINING A FLOW SPEED OF A FLUID FLOWING THROUGH A CARDIAC ASSIST DEVICE, in PCT Pub. No. WO 2019/234167, filed Jun. 6, 2019, titled DETERMINATION APPLIANCE AND METHOD FOR DETERMINING A VISCOSITY OF A FLUID, in U.S. patent application Ser. No. 15/734,519, filed Dec. 2, 2020, titled DETERMINATION APPLIANCE AND METHOD FOR DETERMINING A VISCOSITY OF A FLUID, in PCT Pub. No. WO 2019/234169, filed Jun. 6, 2019, titled ANALYSIS APPARATUS AND METHOD FOR ANALYZING A VISCOSITY OF A FLUID, in U.S. patent application Ser. No. 15/734,489, filed Dec. 2, 2020, titled ANALYSIS APPARATUS AND METHOD FOR ANALYZING A VISCOSITY OF A FLUID, in PCT Pub. No. WO 2019/243582, filed Jun. 21, 2019, titled METHOD AND DEVICE FOR DETECTING A WEAR CONDITION OF A VENTRICULAR ASSIST DEVICE AND FOR OPERATING SAME, AND VENTRICULAR ASSIST DEVICE, and/or in U.S. patent application Ser. No. 17/252,498, filed Jul. 27, 2021, titled METHOD AND DEVICE FOR DETECTING A WEAR CONDITION OF A VENTRICULAR ASSIST DEVICE AND FOR OPERATING SAME, AND VENTRICULAR ASSIST DEVICE, each of which are hereby incorporated by reference herein in their entirety for all purposes and forms a part of this specification.

Various modifications to the implementations described in this disclosure will be readily apparent to those skilled in the art, and the generic principles defined herein can be applied to other implementations without departing from the spirit or scope of this disclosure. Thus, the disclosure is not intended to be limited to the implementations shown herein but is to be accorded the widest scope consistent with the claims, the principles and the novel features disclosed herein. The word “example” is used exclusively herein to mean “serving as an example, instance, or illustration.” Any implementation described herein as “example” is not necessarily to be construed as preferred or advantageous over other implementations, unless otherwise stated. The word “about” may refer to values within ±1%, ±2%, ±3%, ±4%, ±5%, ±10%, ±15%, or other ranges depending on context and as may be understood by one of ordinary skill in the art.

Certain features that are described in this specification in the context of separate implementations also can be implemented in combination in a single implementation. Conversely, various features that are described in the context of a single implementation also can be implemented in multiple implementations separately or in any suitable sub-combination. Moreover, although features can be described above as acting in certain combinations and even initially claimed as such, one or more features from a claimed combination can in some cases be excised from the combination, and the claimed combination can be directed to a sub-combination or variation of a sub-combination.

Similarly, while operations are depicted in the drawings in a particular order, this should not be understood as requiring that such operations be performed in the particular order shown or in sequential order, or that all illustrated operations be performed, to achieve desirable results. Additionally, other implementations are within the scope of the following claims. In some cases, the actions recited in the claims can be performed in a different order and still achieve desirable results.

It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations.

In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

If an exemplary embodiment comprises a “and/or” link between a first feature and a second feature, this is to be read in such a way that the embodiment according to one embodiment has both the first feature and the second feature and according to a further embodiment has either only the first feature or only the second feature.

What is claimed is:

1. A mechanical circulatory support system, comprising:
a circulatory support catheter, comprising a circulatory support device carried by an elongate flexible catheter shaft, the circulatory support device comprising a tubular housing, a motor, and an impeller configured to be rotated by the motor;
an insertion tool having a tubular body and configured to axially movably receive the circulatory support device; and
an introducer sheath, having a tubular body and configured to axially movably receive the insertion tool.

2. The system of claim 1, wherein the impeller is configured to be rotated by the motor via a shaft.

3. The system of claim 2, wherein the circulatory support device comprises an annular polymeric seal around the shaft.

4. The system of claim 2, wherein the circulatory support device comprises a seal around the shaft, the seal comprising a distal radial shaft seal having a distal side configured to face distally toward the impeller and a radially inner lip configured to contact the shaft and to extend from the distal side in a proximal direction toward the motor.

5. The system of claim 4, further comprising a proximal radial shaft seal having a proximal side configured to face proximally toward the motor and a radially inner lip configured to contact the shaft and to extend from the proximal side in a distal direction toward the impeller.

6. The system of claim 1, wherein the impeller is configured to be rotated by the motor via a magnetic coupling.

7. The system of claim 1, wherein the introducer sheath comprises a hub on a proximal end of the introducer sheath, the hub having a feature for preventing axial and optionally rotational movement of the insertion tool.

8. The system of claim 7, the hub comprising one or more hemostatic valves.

9. The system of claim 8, wherein the tubular body of the insertion tool has sufficient collapse resistance to maintain patency when passed through the one or more hemostatic valves of the introducer sheath.

10. The system of claim 7, wherein the hub and a relief bend disposed between the hub and the tubular body of the introducer sheath are configured to axially movably receive the tubular body of the insertion tool.

11. The system of claim 1, wherein the insertion tool comprises a tube with a valve in fluid communication with an inner lumen of the tubular body of the insertion tool configured for flushing with saline.

12. The system of claim 1, the catheter shaft comprising a visual marker spaced proximally from the circulatory support device such that visibility of the visual marker on a proximal side of the introducer sheath indicates the circulatory support device is located within the tubular body of the insertion tool.

13. The system of claim 1, further comprising a first guidewire port on a distal end of the tubular housing of the circulatory support device, a second guidewire port on a sidewall of the tubular housing of the circulatory support device and distal to the impeller, and a third guidewire port on a proximal side of the impeller.

14. The system of claim 13, wherein a distal end of the tubular body of the insertion tool detachably connects to a guidewire aid configured to facilitate entry of a guidewire through the first guidewire port.

15. The system of claim 13, wherein a removable guidewire guide tube enters the first guidewire port on the distal end of the tubular housing, exits the tubular housing via the second guidewire port on the sidewall of the tubular housing distal to the impeller, reenters the tubular housing via the third guidewire port on the proximal side of the impeller, and extends proximally into the catheter shaft.

16. The system of claim 15, wherein the tubular body of the insertion tool is configured to receive the circulatory support device with the removable guidewire guide tube.

17. The system of claim 16, wherein the tubular body of the insertion tool and the guidewire guide tube are transparent.

18. The system of claim 1, wherein the tubular body of the insertion tool has a length within a range of from about 85 mm to about 160 mm and an inside diameter within a range of from about 4.5 mm to about 6.5 mm.

19. The system of claim 1, the tubular housing of the circulatory support device comprising an inlet tube coupled with a motor housing, the inlet tube having one or more distal pump inlets and one or more proximal pump outlets, and the impeller adjacent the one or more proximal pump outlets.

20. The system of claim 1, wherein the system does not require purging.

21. The system of claim 1, wherein the introducer sheath is a 16 French (Fr) sheath.

22. The system of claim 1, wherein the circulatory support device is configured to provide a flow rate of blood of about 4.0 liters per minute (l/min) for about 6 hours.

23. The system of claim 1, wherein the insertion tool comprises a hemostatic valve.

24. The system of claim 1, wherein the insertion tool comprises a plug disposed at a proximal end of the insertion tool configured to connect to a sterile shield sleeve.

25. The system of claim 1, wherein the insertion tool comprises a locking mechanism, the locking mechanism comprising a recess configured to accept a locking pad configured to releasably lock with the circulatory support catheter.

26. The system of claim 25, wherein the insertion tool comprises a housing surrounding at least a portion of the locking mechanism, the housing comprising opposing first inner surface walls spaced farther than opposing second inner surface walls, wherein the at least a portion of the locking mechanism comprises radially outwardly extending tabs, and wherein the housing is configured to rotate to inwardly compress the tabs to prevent axial movement of the circulatory support catheter.

27. The system of claim 26, wherein inward compression of the tabs of the locking mechanism compresses the locking pad against the circulatory support catheter.

28. A mechanical circulatory support system, comprising:
an elongate flexible catheter shaft, having a proximal end and a distal end;
a circulatory support device carried by the distal end of the catheter shaft, the circulatory support device comprising a tubular housing, a motor, and an impeller configured to be rotated by the motor;
wherein the circulatory support device is configured to provide a flow rate of blood of up to about 4.0 liters per minute (l/min) for about 6 hours without purging of the system.

29. The system of claim 28, wherein the impeller is configured to be rotated by the motor via a shaft.

30. The system of claim 29, wherein the circulatory support device comprises an annular polymeric seal around the shaft.

31. The system of claim 29, wherein the circulatory support device comprises a seal around the shaft, the seal comprising a distal radial shaft seal having a distal side configured to face distally toward the impeller and a radially inner lip configured to contact the shaft and to extend from the distal side in a proximal direction toward the motor.

32. The system of claim 31, further comprising a proximal radial shaft seal having a proximal side configured to face proximally toward the motor and a radially inner lip configured to contact the shaft and to extend from the proximal side in a distal direction toward the impeller.

33. The system of claim 28, wherein the impeller is configured to be rotated by the motor via a magnetic coupling.

34. The system of claim 28, further comprising an insertion tool having a tubular body and configured to axially movably receive the circulatory support device.

35. The system of claim 34, further comprising an introducer sheath having a tubular body and configured to axially movably receive the insertion tool.

36. The system of claim 28, further comprising a controller that does not include a purging component.

37. The system of claim 36, wherein the controller does not include a cassette or a port for purging.

38. The system of claim 28, the impeller comprising a blade having a proximal vane section with a wave-shaped vane curvature defined by one or more curved portions of a skeleton line of the blade.

39. The system of claim 28, the tubular housing of the circulatory support device comprising an inlet tube with a main body, wherein the main body comprises:
a first attachment section at a first end of the main body configured to attach the inlet tube to a head unit of the circulatory support device; and
a second attachment section at a second end of the main body, wherein the first attachment section is configured to connect to the head unit in a form-locking and/or force-locking manner, wherein the main body further comprises a structural section comprising at least one stiffening recess between the first attachment section and the second attachment section.

40. The system of claim 28, the impeller comprising a blade having at least one blade section having a wavy blade curvature.

41. The system of claim 40, wherein the tubular housing of the circulatory support device comprises an inlet tube having an inlet and an outlet, and wherein the outlet and the blade section having the wavy blade curvature at least partially axially overlap.

42. The system of claim 28, the impeller comprising:
a blade element having a profile with camber lines, wherein a curvature of each of the camber lines when unwound into a plane increases along the axis of rotation in a direction starting from the pump intake section towards the outlet opening to an inflection point at which a blade angle (β) of the blade element is at a maximum, and wherein the curvature of each of the camber lines decreases after the inflection point, and
wherein, in a region of the impeller located radially relative to an axis of rotation of the impeller and having a blade height SH of the blade element defined relative to a maximum blade height SHMAX such that 25%≤SH/SHMAX≤100%, the inflection point of each of the camber lines is located in a region of an upstream edge of an outlet opening of an inlet tube of the tubular housing.

43. The system of claim 28, further comprising:
the tubular housing comprising an outlet opening configured to facilitate outflow of the blood; and
a diffuser configured to couple with the tubular housing, wherein, in an operating position, the diffuser is configured to guide the blood transversely to the outlet opening after the blood has passed through the outlet opening.

44. The system of claim 28, the tubular housing comprising an inlet tube having a mesh section with a mesh structure formed from at least one mesh wire.

45. The system of claim 44, wherein the mesh section is bent at an obtuse angle at a bending point.

46. The system of claim 28, the tubular housing comprising an inlet tube for conveying the blood through the inlet tube, and a reduced diameter section at a distal end of the inlet tube.

47. The system of claim 28, the tubular housing comprising:
a feed head portion comprising at least one introduction opening for receiving the fluid flow into the feed line; and
a contoured portion disposed adjacent to the feed head portion and comprising an inner surface contour, wherein the inner surface contour comprises a first inner diameter at a first position, a second inner diameter at a second position, and a third inner diameter at a third position, wherein the first inner diameter is greater than the second inner diameter, wherein the third inner diameter is greater than the second inner diameter, wherein the first inner diameter comprises a maximum inner diameter of the contoured portion and the second inner diameter comprises a minimum inner diameter of the contoured portion, wherein the inner surface contour comprises a rounded portion at the second position, wherein the contoured portion comprises a first inner radius at the first position and a second inner radius at the second position, wherein the second inner radius is at most one fifth smaller than the first inner radius, and wherein the second position is located between the third position and the first position.

48. The system of claim 28, the tubular housing comprising a radiopaque marker at a distal end of the tubular housing.

49. The system of claim 28, the tubular housing comprising an inlet tube with a nose piece at a distal end of the inlet tube, the nose piece comprising a radiopaque marker.

50. The system of claim 34, wherein the insertion tool comprises a hemostatic valve.

51. The system of claim 34, wherein the insertion tool comprises a locking mechanism, the locking mechanism comprising a recess configured to accept a locking pad configured to releasably lock with the catheter shaft.

52. The system of claim 51, wherein the insertion tool comprises a housing surrounding at least a portion of the locking mechanism, the housing comprising opposing first inner surface walls spaced farther than opposing second inner surface walls, wherein the at least a portion of the locking mechanism comprises radially outwardly extending tabs, and wherein the housing is configured to rotate to inwardly compress the tabs to prevent axial movement of the catheter shaft.

53. The system of claim 52, wherein inward compression of the tabs of the locking mechanism compresses the locking pad against the catheter shaft.

54. The system of claim 35, wherein the introducer sheath comprises a hub on a proximal end of the introducer sheath, the hub having a feature for preventing axial and optionally rotational movement of the insertion tool.

55. The system of claim 54, the hub comprising one or more hemostatic valves.

56. The system of claim 55, wherein the tubular body of the insertion tool has sufficient collapse resistance to maintain patency when passed through the one or more hemostatic valves of the introducer sheath.

57. The system of claim 54, wherein the hub and a relief bend disposed between the hub and the tubular body of the introducer sheath are configured to axially movably receive the tubular body of the insertion tool.

58. The system of claim 34, wherein the insertion tool comprises a tube with a valve in fluid communication with an inner lumen of the tubular body of the insertion tool configured for flushing with saline.

59. The system of claim 28, further comprising a first guidewire port on a distal end of the tubular housing of the circulatory support device, a second guidewire port on a sidewall of the tubular housing of the circulatory support device and distal to the impeller, and a third guidewire port on a proximal side of the impeller.

60. The system of claim 59, wherein a distal end of the tubular body of the insertion tool detachably connects to a guidewire aid configured to facilitate entry of a guidewire through the first guidewire port.

61. The system of claim 59, wherein a removable guidewire guide tube enters the first guidewire port on the distal end of the tubular housing, exits the tubular housing via the second guidewire port on the sidewall of the tubular housing distal to the impeller, reenters the tubular housing via the third guidewire port on the proximal side of the impeller, and extends proximally into the catheter shaft.

62. The system of claim 61, wherein the tubular body of the insertion tool is configured to receive the circulatory support device with the removable guidewire guide tube.

63. The system of claim 62, wherein the tubular body of the insertion tool and the guidewire guide tube are transparent.

64. The system of claim 34, wherein the insertion tool comprises a plug disposed at a proximal end of the insertion tool configured to connect to a sterile shield sleeve.
